(19) United States
(12) Patent Application Publication
(10) Pub. No.: US 2006/0270628 A1
(43) Pub. Date: Nov. 30, 2006

(54) Antiinfective 1,2,3-triazole derivatives, process for their preparation and pharmaceutical compositions containing them

(76) Inventors: Jagattaran Das, Hyderabad (IN); Selvakumar Natesan, Hyderabad (IN); Sanjay Trehan, Hyderabad (IN); Javed Iqbal, Hyderabad (IN); Sitaram Kumar Magadi, Hyderabad (IN)

Correspondence Address: Ms. Milagros A. Cepeda, Patent Counsel;Dr. Reddy's Laboratories, Inc., 200 Somerset Corporate Boulevard-7th Floor, Bridgewater, NJ, 08807, US

(21) Appl. No.: 10/554,472

(22) PCT Filed: Apr. 29, 2004
(86) PCT No.: PCT/IB04/01303

§ 371 Date: Oct. 24, 2005

(30) Foreign Application Priority Data
Apr. 29, 2003 (IN) 365/MAS/2003

(51) Int. Cl. A61K 31/695; A61K 31/655; A61K 31/4192; C07D 403/02; C07F 7/02

(52) U.S. Cl. 514/63; 514/151; 514/227.5; 514/359; 544/60; 548/255; 548/110

(57) ABSTRACT

The present invention relates to novel triazole compounds of formula (I), thie pharmaceutically acceptable salts and their pharmaceutical compositions, where all symbols have meaning as defined in the description, for use in the treatment of bacterial infections.

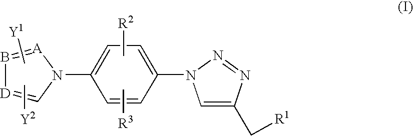

### FIELD OF THE INVENTION

The present invention relates to novel triazole compounds of formula (I), 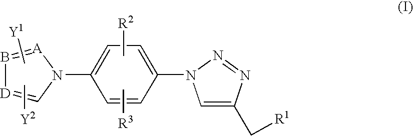
where R¹ represents halogen, azido, thioalcohol, isothiocyanate, hydroxy, isoindole-1,3-dione, substituted or unsubstituted (C1-C20)alkylsulfonyloxy, arylsulfonyloxy, (C1-C20)acyloxy group, NHR⁴ where R⁴ represents hydrogen, substituted or unsubstituted groups selected from (C1-C20)acyl, thio(C1-C20)acyl, (C1-C20)alkoxycarbonyl, (C3-C20)cycloalkoxycarbonyl, (C3-C20)cycloalkoxythiocarbonyl, (C2-C20)alkenyloxycarbonyl, (C2-C20)alkenylcarbonyl, heteroaryl, aryloxycarbonyl, heteroarylcarbonyl, heteroarylthiocarbonyl, (C1-C20)alkoxythiocarbonyl, (C2-C20)alkenyloxythiocarbonyl, aryloxythiocarbonyl, —C(═O)—C(═O)—(C1-C20)alkyl, —C(═O)—C(═O)-aryl, —C(═O)—C(═O)—(C1-C20)alkoxy, —C(═O)—C(═O)—aryloxy, —C(═O)—C(═S)—(C1-C20)alkyl, —C(═O)—C(═S)-aryl, —C(═S)—S—(C1-C20)alkyl, —C(═S)—NH₂, —C(═S)—NH—(C1-C20)alkyl, —C(═S)—N—((C1-C20)alkyl)₂, —C(═S)—NH—(C2-C20)alkenyl, —C(═S)—C(═O)—(C1-C20)alkoxy, —C(═S)—C(═O)-aryloxy, —C(═S)—O—C(═O)—(C1-C20)alkyl, —C(═S)—C(═S)—(C1-C20)alkyl, —C(═S)—C(═S)-aryl, —C(═S)—NH—C(═O)-aryl, —C(═S)—NH-aralkyl, —C(═S)—NH-heteroaralkyl, —C(═NH)—NH₂, —C(═NH)—(C1-C20)alkyl, —C(═NH)-aryl, —S(O)₂(C1-C20)alkyl, —S(O)₂aryl, thiomorpholinylthiocarbonyl, pyrrolidinylthiocarbonyl or —C(═S)—N(R′R″), where R′ and R″ together form a substituted or unsubstituted 5 or 6 member heterocycle ring containing nitrogen and optionally having one or two additional hetero atoms selected from O or S; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, (C1-C20)alkylaminocarbonyl, arylcarbonylamino(C1-C20)alkyl, (C1-C20)alkylcarbonyloxy(C1-C20)alkyl, amino(C1-C20)alkyl, mono(C1-C20)alkylamino, di(C1-C20)alkylamino, (C1-C20)alkoxy, or any one or two of Y¹or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH; their pharmaceutically acceptable salts and pharmaceutical compositions containing them.

### BACKGROUND OF THE INVENTION

Since the discovery of penicillin, pharmaceutical companies have produced more than one hundred antibacterial agents to combat a wide variety of bacterial infections. In the past several years, there has been rapid emergence of bacterial resistance to several of these antibiotics. The multidrug resistance among these bacterial pathogens may also be due to mutation leading to more virulent clinical isolation; the most disturbing milestone has been the acquisition of resistance to vancomycin, an antibiotic generally regarded as the agent of last resort for serious Gram-positive infections. This growing multidrug resistance has recently rekindled interest in the search for new structural class of antibiotic that inhibit or kill these bacteria possibly by novel mechanisms.

A problem of larger dimension is the increasing incidence of the more virulent, methicillin-resistant Staphylococcus aureas (MRSA) among clinical isolates found worldwide. As with vancomycin resistant organisms, many MRSA strains are resistant to most of the known antibiotics, but MRSA strains have remained sensitive to vancomycin. However, in view of the increasing reports of vancomycin resistant clinical isolates and growing problem of bacterial resistance, there is an urgent need for new molecular entities effective against the emerging and currently problematic Gram-positive organisms.

Recently, several oxazolidinones have been discovered, which inhibit protein synthesis by binding to the 50S-ribosomal subunit which is close to the site to which chloramphenicol and lincomycin bind but their mode of action is mechanistically distinct from these two antibiotics.

Various 1,2,3-triazoles, 1,2,4-triazoles and benzotriazoles have been reported to show various biological activities and have therefore found applications in medicinal chemistry.

Some of the Literature Refences are:

(a) Chem. Pharm. Bull. 48(12), 1935-1946 (2000) discloses the triazoles of formula (ia) and (ib), which are reported as antifungal agents,
(b) U.S. Pat. No. 6,054,471 discloses fluorinated triazoles of the formula (ii), which are reported for the treatment of neuropathic pain and associated hyperalgesia, including trigeminal and herpectic neuralgia, diabetic neuropathic pain, migraine, causalgia and deafferentation syndromes such as brachial plexus avulsion,

(c) J. Med. Chem., 2843, 1991 discloses compound of formula (iii), which is an anticoccidiostat and also been found to have antiproliferative activity in several disease models and to posses antimetastatic activity in a model of ovarian cancer progression,

(d) J. Heterocycl. Chem., 609, 1989 discloses compound of formula (iv), which is reported for anti-inflammatory effects,

(e) EPO publication no 0304221 A2 discloses compounds of formula (v), which are reported as antiproliferative reagents.

(f) PCT publication no. WO03/059894 (by Dr. Reddy's Laboratories Ltd.) discloses 1,2,3-triazoles as antibacterial agents.

The novel triazole compound of the present invention is useful for the treatment of various infections

### SUMMARY OF THE INVENTION

According to one aspect of the present invention, there is provided novel triazole compounds of the general formula (I) as defined above, their pharmaceutically acceptable salts and their pharmaceutical compositions containing them.

Another aspect fo the present invention provides process for the preparation of novel triazole compounds of the formula (I).

Yet another aspect of the pesent invention provides the use of novel compounds of formula (I) or its pharmaceutical compositions in the treatment of bacterial infections.

### DETAILED DESCRIPTION OF THE INVENTION

The present invention relates to compounds having the general formula (I), 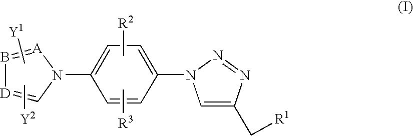
where R¹ represents halogen, azido, thioalcohol, isothiocyanate, hydroxy, isoindole-1,3-dione, substituted or unsubstituted (C1-C20)alkylsulfonyloxy, arylsulfonyloxy, (C1-C20)acyloxy group, NHR⁴ where R⁴ represents hydrogen, substituted or unsubstituted groups selected from (C1-C20)acyl, thio(C1-C20)acyl, (C1-C20)alkoxycarbonyl, (C3-C20)cycloalkoxycarbonyl, (C3-C20)cycloalkoxythiocarbonyl, (C2-C20)alkenyloxycarbonyl, (C2-C20)alkenylcarbonyl, heteroaryl, aryloxycarbonyl, heteroarylcarbonyl, heteroarylthiocarbonyl, (C1-C20)alkoxythiocarbonyl, (C2-C20)alkenyloxythiocarbonyl, aryloxythiocarbonyl, —(═O)—C(═O)—(C1-C20)alkyl, —C(═O)—C(═O)-aryl, —C(═O)—C(═O)—(C1-C20)alkoxy, —C(═O)—C(═O)-aryloxy, —C(═O)—C(═S)—(C1-C20)alkyl, —C(═O)—C(═S)-aryl, —C(═S)—S—(C1-C20)alkyl, —C(═S)—NH₂, —C(═S)—NH—(C1-C20)alkyl, —C(═S)—N—((C1-C20)alkyl)₂, —C(═S)—NH—(C2-C20)alkenyl, —C(═S)—C(═O)—(C1-C20)alkoxy, —C(═S)—C(═O)-aryloxy, —C(═S)—O—C(═O)—(C1-C20)alkyl, —C(═S)—C(═S)—(C1-C20)alkyl, —C(═S)—C(═S)-aryl, —C(═S)—NH—C(═O)-aryl, —C(═S)—NH-aralkyl, —C(═S)—NH-heteroaralkyl, —C(═NH)—NH₂, —C(═NH)—(C1-C20)alkyl, —C(═NH)-aryl, —S(O)₂(C1-C20)alkyl, —S(O)₂aryl, thiomorpholinylthiocarbonyl, pyrrolidinylthiocarbonyl or —C(═S)—N(R′R″), where R′ and R″ together form a substituted or unsubstituted 5 or 6 member heterocycle ring containing nitrogen and optionally having one or two additional hetero atoms selected from O or S; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, (C1-C20)alkylaminocarbonyl, arylcarbonylamino(C1-C20)alkyl, (C1-C20)alkylcarbonyloxy(C1-C20)alkyl, amino(C1-C20)alkyl, mono(C1-C20)alkylamino, di(C1-C20)alkylamino, (C1-C20)alkoxy, or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH; their pharmaceutically acceptable salts and pharmaceutical compositions containing them.

Suitable groups represented by R⁴ are described as (C1-C20)acyl group such as —C(═O)H, —C(═O)CH₃, —C(═O)CH₂CH₃, —C(═O)(CH₂)CH₃, —C(═O)(CH₂)₃CH₃, —C(═O)(CH₂)₄CH₃, —C(═O)(CH₂)₅CH₃, —C(═O)Ph and the like, the (C1-C20)acyl group may be substituted; thio(C1-C20)acyl group such as —C(═S)H, —C(═S)CH₃, —C(═S)CH₂CH₃, —C(═S)Ph and the like, the thio(C1-C20)acyl group may be substituted; (C1-C20)alkoxycarbonyl group containing (C1-C20)alkyl group which may be linear or branched such as methoxycarbonyl, ethoxycarbonyl, propoxycarbonyl, isopropoxycarbonyl and the like, the (C1-C20)alkoxycarbonyl group may be substituted; (C3-C20)cycloalkoxycarbonyl such as cyclopropoxycarbonyl, cyclobutoxycarbonyl and the like, the (C3-C20)cycloalkoxycarbonyl may be substituted; (C3-C20)cycloalkoxythiocarbonyl such as cyclopropoxythiocarbonyl, cyclobutoxythiocarbonyl and the like, the (C3-C20)cycloalkoxythiocarbonyl may be substituted; (C2-C20)alkenylcarbonyl such as ethenylcarbonyl, propenylcarbonyl, butenylcarbonyl and the like, the (C2-C20)alkenylcarbonyl may be substituted; heteroaryl group such as pyridyl, furyl, thiophenyl, benzothiazoyl, purinyl, benzimidazoyl, pyrimidinyl, tetrazolyl and the like, the heteroaryl group may be substituted; heteroarylcarbonyl such as pyridylcarbonyl, furylcarbonyl, thiophenylcarbonyl, benzothiazoylcarbonyl, benzimidazoylcarbonyl, pyrimidinylcarbonyl, pyridazinecarbonyl, pyrimidinecarbonyl, pyrazinecarbonyl, tetrazolylcarbonyl and the like, the heteroarylcarbonyl group may be substituted, heteroarylthiocarbonyl such as pyridylthiocarbonyl, furylthiocarbonyl, thiophenylthiocarbonyl, benzothiazoylthiocarbonyl, benzimidazoylthiocarbonyl, pyrimidinylthiocarbonyl, pyridazinethiocarbonyl, pyrimidinethiocarbonyl, pyrazinethiocarbonyl, tetrazolylthiocarbonyl and the like, the heteroarylthiocarbonyl may be substituted, (C2-C20)alkenyloxycarbonyl group such as ethenyloxycarbonyl, propenyloxycarbonyl, butenyloxycarbonyl and the like, the (C2-C20)alkenyloxycarbonyl may be substituted; aryloxycarbonyl group such as phenoxycarbonyl, benzyloxycarbonyl group and the like, the aryloxycarbonyl group may be substituted; (C1-C20)alkoxythiocarbonyl group such as CH₃O—C(═S)—, C₂H₅O—C(═S)—C₃H₇O—C(═S)— and the like, (C1-C20)alkoxythiocarbonyl group may be substituted; (C2-C20)alkenyloxythiocarbonyl group such as ethenyloxythiocarbonyl, propenyloxythiocarbonyl, butenyloxythiocarbonyl and the like, the (C2-C20)alkenyloxythiocarbonyl group may be substituted; aryloxythiocarbonyl group such as (phenyl)O—C(═S)—, (benzyl)O—C(═S)— and the like, which may be substituted; —C(═O)—C(═O)—(C1-C20)alkyl group such as —C(═O)—C(═O)methyl, —C(═O)—C(═O)ethyl, —C(═O)—C(═O)propyl and the like, which may be substituted; —C(═O)—C(═O)-aryl group such as —C(═O)—C(═O)phenyl, —C(═O)—C(═O)naphthyl and the like, which may be substituted; —C(═O)—C(═O)—(C1-C20)alkoxy group such as —C(═O)—C(═O)methoxy, —C(═O)—C(═O)ethoxy, —C(═O)—C(═O)propyloxy and the like, which may be substituted; —C(═O)—C(═O)-aryloxy group such as —C(═O)—C(═O)phenyloxy, —C(═O)—C(═O)benzyloxy, which may be substituted; —C(═O)—C(═S)—(C1-C20)alkyl group such as —C(═O)—C(═S)-methyl, —C(═O)—C(═S)-ethyl, —C(═O)—C(═S)-propyl, —C(═O)—C(═S)-butyl and the like, which may be substituted; —C(═O)—C(═S)-aryl group such as —C(═O)—C(═S)-phenyl, —C(═O)—C(═S)-naphthyl and the like, which may be substituted; —(C═S)—S—(C1-C20)alkyl such as —(C═S)—S-methyl, —(C═S)—S-ethyl, —(C═S)—S-propyl and the like, which may be substituted; —(C═S)—NH₂, which may be substituted; —(C═S)—NH—(C1-C20)alkyl such as —(C═S)—NH-methyl, —(C═S)—NH-ethyl, —(C═S)—NH-propyl and the like, which may be substituted; —C(═S)—N—((C₁-C₆)alkyl)₂ such as —C(═S)—N-(methyl)₂, —C(═S)—N-(ethyl)₂, —C(═S)—N-(propyl)₂ and the like, which may be substituted; —C(═S)—NH—(C2-C20)alkenyl such as —C(═S)—NH-ethenyl, —C(═S)—NH-propenyl, —C(═S)—NH-butenyl and the like, which may be substituted; —(C═S)—(C═O)—(C1-C20)alkoxy such as —(C═S)—(C═O)-methoxy, —(C═S)—(C═O)-ethoxy, —(C═S)—(C═O)-propoxy and the like, which may be substituted; —(C═S)—(C═O)-aryloxy such as —(C═S)—(C═O)-phenyloxy, —(C═S)—(C═O)-naphthyloxy and the like, which may be substituted; —C(═S)—O—(C═O)—(C1-C20)alkyl such as —C(═S)—O—(C═O)-methyl, —C(═S)—O—(C═O)-ethyl, —C(═S)—O—(C═O)-propyl and the like, which may be substituted; —C(═S)—C(═S)—(C1-C20)alkyl group such as —C(═S)—C(═S)methyl, —C(═S)—C(═S)ethyl, —C(═S)—C(═S)propyl and the like, which may be substituted; —C(═S)—C(═S)aryl group such as —C(═S)—C(═S)phenyl, —C(═S)—C(═S)naphthyl and the like, which may be substituted; —C(═S)—NH—C(═O)-aryl group such as —C(═S)—NH—C(═O)-phenyl, —C(═S)—NH—C(═O)-naphthyl and the like, —C(═S)—NH—C(═O)-aryl group may be substituted; —C(═S)—NH-aralkyl group such as —C(═S)—NH-benzyl, —C(═S)—NH-phenethyl, —C(═S)—NH—C₆H₅CH₂CH₂CH₂, —C(═S)—NH-naphthylmethyl and the like, —C(═S)—NH-aralkyl group may be substituted; —C(═S)—NH-heteroaralkyl such as —C(═S)—NH-pyridinemethyl, —C(═S)—NH-furanmethyl, —C(═S)—NH-thiophenylenemethyl, —C(═S)—NH-benzothiazolemethyl, —C(═S)—NH-benzimidazolemethyl, —C(═S)—NH-pyrimidinemethyl, —C(═S)—NH-pyrimidinemethyl, —C(═S)—NH-pyrazinemethyl, —C(═S)—NH-tetrazolemethyl and the like, where —C(═S)—NH-aralkyl group may be substituted; —C(═NH)—NH₂, which may be substituted; —C(═NH)—(C1-C20)alkyl such as —C(═NH)-methyl, —C(═NH)-ethyl, —C(═NH)-propyl and the like, which may be substituted; —C(═NH)-aryl such as —C(═NH)-phenyl, —C(═NH)-naphthyl and the like, which may be substituted; S(O)₂—(C1-C20)alkyl such as S(O)₂-methyl, S(O)₂-ethyl, S(O)₂-propyl, S(O)₂-isopropyl, S(O)₂-butyl, S(O)₂-isobutyl and the like, which may be substituted; S(O)₂-aryl such as S(O)₂-phenyl, S(O)₂-naphthyl and the like, which may be substituted; thiomorpholinylthiocarbonyl, which may be substituted; pyrrolidinylthiocarbonyl, which may be substituted; or —C(═S)—N(R′R″) where R′R″ are as defined above.

A 5 or 6 member heterocycle ring formed by R′ & R″, containing nitrogen, optionally having one or two additional heteroatoms selected from oxygen, nitrogen or sulfur, is selected from pyrrolidinyl, pyrrolyl, morpholinyl, thiomorpholinyl, benzothiazole, benzoimidazolyl, pyridinyl, pyridazinyl, pyrimidinyl, pyrazinyl and the like, the heterocycle may be substituted.

When the groups represented by R⁴, R^{4a}, R^{4b}, 4^{4c}, 4^{4d}, R^{4e}, R, R⁷ and heterocycles formed by R′ and R″ are substituted, the substituents may be selected from halogen atom such as chlorine, fluorine, bromine and iodine; hydroxy, amino, cyano, nitro, (C1-C20)alkyl, which is as defined as earlier; hydroxy(C1-C20)alkyl, in which (C1-C20)alkyl groups is as defined earlier; (C1-C20)alkoxy group such as methoxy, ethoxy, propoxy and the like; ═O, ═S, aryl group such as phenyl, naphthyl and the like, hydroxyaryl such as hydroxyphenyl, hydroxynaphthyl and the like, pyridyl, mono(C1-C20)alkylamino such as methylamino, ethylamino, propylamino and the like; di(C1-C20)alkylamino such as dimethylamino, diethylamino, dipropylamino and the like; (C1-C20)acyl group such as —C(═O)H, —C(═O)CH₃, —C(═O)CH₂CH₃, —C(═O)(CH₂)₂CH₃, —C(═O)(CH₂)₃CH₃, —C(═O)(CH₂)₄CH₃, —C(═O)(CH₂)₅CH₃, —C(═O)Ph and the like; thio(C1-C20)acyl group such as —C(═S)H, —C(═S)CH₃, —C(═S)CH₂CH₃, —C(═S)Ph and the like; (C1-C20)alkoxycarbonyl group such as methoxycarbonyl, ethoxycarbonyl, propoxycarbonyl, butoxycarbony, tert-butoxycarbonyl(BOC) and the like; (C1-C20)alkoxyaryl group such as methoxyaryl, ethoxyaryl, propoxyaryl, iso-propoxyaryl, butoxyaryl and the like, where aryl group is as defined above or carboxylic acid or its derivatives selected from amides and esters such as CONH₂, CONHMe, CONMe₂, CONHEt, CONEt₂, CONHPh, COOCH₃, COOC₂H₅ or COOC₃H₇.

Suitable groups represented by R² and R³ may be selected from hydrogen, halogen atom such as fluorine, chlorine or bromine; substituted or unsubstituted (C1-C20)alkyl group such as methyl, ethyl, n-propyl, isopropyl, n-butyl, iso-butyl, t-butyl, n-pentyl, iso-pentyl, n-hexyl and the like; halo(C1-C20)alkyl group such as halomethyl, haloethyl, halopropyl, trihalomethyl and the like, wherein the halo group is selected from fluorine, chlorine, bromine or iodine; (C1-C20)alkoxy group such as methyl, ethyl, propyl and the like; aryl group such as phenyl, naphthyl and the like; heteroaryl groups such as pyridyl, thienyl, furyl, pyrrolyl, oxazolyl, thiazolyl, pyrazolyl, imidazolyl and the like; cyano, nitro; OR^{a} where R^{a} represents substituted or unsubstituted (C1-C20)alkyl group such as methyl, ethyl, propyl, isopropyl and the like.

Suitable substitutents on R² and R³ are selected from hydroxy, halogen, nitro, amino, (C1-C20)alkyl, (C1-C20)alkoxy, ═O, ═S, cyano group, or carboxylic acid or its derivatives. These groups are as defined above.

Suitable substitutents on R^{a} are selected from hydroxy, halogen, nitro, amino, (C1-C20)alkyl, (C1-C20)alkoxy, cyano group, or carboxylic acid or its derivatives. These groups are as defined above.

Suitable groups represented by Y¹ and Y² are selected from hydrogen, cyano, nitro, formyl, hydroxy, amino, halogen such as fluorine, chlorine, bromine or iodine; substituted or unsubstituted (C1-C20)alkyl such as methyl, ethyl, n-propyl, isopropyl, n-butyl, iso-butyl, t-butyl and the like, which may be substituted; hydroxy(C1-C20)alkyl such as hydroxymethyl, hydroxyethyl, hydroxypropyl and the like, which may be substituted; dihydroxy(C1-C20)alkyl such as dihydroxymethyl, dihydroxyethyl, dihydroxypropyl and the like, which may be substituted; (C1-C20)alkoxy(C1-C20)alkyl group such as methoxymethyl, methoxyethyl, ethoxyethyl, ethoxymethyl, methoxypropyl, propoxymethyl, propoxyethyl and the like, which may be substituted; (C1-C20)alkylcarbonyl group such as methylcarbonyl, ethylcarbonyl and the like, which may be substituted; (C1-C20)alkoxycarbonyl group such as methoxycarbonyl, ethoxycarbonyl and the like, which may be substituted; carboxy(C1-C20)alkyl such as —CH₂—COOH, —CH₂—CH₂—COOH and the like, which may be substituted; (C1-C20)alkylsulfonyl group such as methylsulfonyl, ethylsulfonyl and the like, which may be substituted; (C1-C20)alkylcarbonylamino(C1-C20)alkyl groups such as methylcarbonylaminomethyl, ethylcarbonylaminomethyl, methylcarbonylaminoethyl and the like, which may be substituted; arylcarbonylamino(C1-C20)alkyl such as phenylcarbonylaminomethyl, phenylcarbonylaminoethyl and the like, which may be substituted; (C1-C20)alkyl aminocarbonyl group such as methylaminocarbonyl, ethylaminocarbonyl, propylaminocarbonyl and the like, which may be substituted; (C1-C20)alkylcarbonyloxy(C1-C20)alkyl group such as methylcarbonyloxymethyl, ethylcarbonylxoymethyl, methylcarbonyloxyethyl, propylcarbonyloxymethyl, propylcarbonyloxyethyl, propylcarbonyloxypropyl and the like, which may be substituted; amino(C1-C20)alkyl such as aminomethyl, aminoethyl, aminopropyl and the like, which may be substituted; mono(C1-C20)alkylamino such as methylamino, ethylamino, propylamino and the like, which may be substituted; di(C1-C20)alkylamino such as dimethylamino, diethylamino, dipropylamino and the like, which may be substituted; (C1-C20)alkoxy group such as methoxy, ethoxy, propoxy, isopropoxy and the like, which may be substituted; Any of Y¹ or Y² may also represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives may be amides or esters. Exemplary carboxylic acid groups include CONH₂, CONHMe, CONMe₂, CONHEt, CONEt₂, CONHPh, COOCH₃, COOC₂H₅ or COOC₃H₇.

When the groups represented by Y¹ and Y² are substituted, the substituents may be selected from hydroxy, nitro, cyano, amino, tert-butyldimethylsilyloxy (TBSO), halogen atom, (C1-C20)alkyl, (C1-C20)alkoxy, (C3-C20)cycloalkyl, aryl, benzyloxy, acyl or acyloxy group such as formyloxy, acetyloxy and the like, carboxylic acid or its esters. The groups are as defined above.

When the groups represented by R^{c} and R^{d} as defined below are substituted, the substituents are selected from halogen, hydroxy, nitro, amino, cyano, (C1-C20)alkyl or (C1-C20)alkoxy. (C1-C20)alkyl and (C1-C20)alkoxy are as defined above.

The groups (C1-C20)alkyl, (C1-C20)alkoxy, aryl, heteroaryl and aralkyl group defined for R′″ are as defined earlier.

When the suitable cites of the above defined groups are substituted, mono, di or tri substitutions are possible.

One aspect of the present invention provides compounds of the formula (I),

R¹ represents NHR⁴ where R⁴ represents (C1-C20)acyl, C1-C20)alkoxycarbonyl;

R² and R³ may be same or different and independently represent hydrogen, halogen atom, (C1-C20)alkyl group, halo(C1-C20)alkyl;

Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, (C1-C20)alkylaminocarbonyl, arylcarbonylamino(C1-C20)alkyl, (C1-C20)alkylcarbonyloxy(C1-C20)alkyl, amino(C1-C20)alkyl, mono(C1-C20)alkylamino, di(C1-C20)alkylamino, (C1-C20)alkoxy, or any one or two of Y¹ or Y² may represent substituted or unsubstituted —CH═NOR′″, wherein R′″ represents hydrogen and (C1-C20)alkyl group, carboxylic acid or its derivatives; their pharmaceutically acceptable salts.

Another aspect of the compound of the formula (I), R¹ represents NHR⁴ where R⁴ represents (C1-C20)acyl, C1-C20)alkoxycarbonyl;

R² and R³ may be same or different and independently represent hydrogen, halogen atom, halo(C1-C20)alkyl;

Y¹ and Y² may be same or different and independently represent hydrogen, cyano, halogen, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkylcarbonylamino(C1-C20)alkyl, (C1-C20)alkylaminocarbonyl, amino(C1-C20)alkyl, mono(C1-C20)alkylamino, di(C1-C20)alkylamino, (C1-C20)alkoxy, or any one or two of Y¹ or Y² may represent substituted or unsubstituted —CH═NOH, carboxylic acid or its derivatives. The substituents on Y¹ and Y² may be selected from hydroxy, cyano, amino, (C1-C20)alkyl, (C1-C20)alkoxy, acyl, carboxylic acid or its esters; their pharmaceutically acceptable salts.

Another aspect of the present invention provides compound of the formula (I),₁₃ R¹ represents NHR⁴ where R⁴ represents thio(C1-C20)acyl, (C1-C20)alkoxythiocarbonyl,

R² and R³ may be same or different and independently represent hydrogen, halogen atom, (C1-C20)alkyl group, halo(C1-C20)alkyl;

Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, (C1-C20)alkylaminocarbonyl, arylcarbonylamino(C1-C20)alkyl, (C1-C20)alkylcarbonyloxy(C1-C20)alkyl, amino(C1-C20)alkyl, mono(C1-C20)alkyl amino, di(C1-C20)alkylamino, (C1-C20)alkoxy, or any one or two of Y¹ or Y² may represent substituted or unsubstituted —CH—NOR′″, wherein R′″ represents hydrogen and (C1-C20)alkyl group, carboxylic acid or its derivatives; their pharmaceutically acceptable salts.

In another aspect of the compound of the formula (I),_R¹ represents NHR⁴ where R⁴ represents thio(C1-C20)acyl, (C1-C20)alkoxythiocarbonyl,

R² and R³ may be same or different and independently represent hydrogen, halogen atom, halo(C1-C20)alkyl;

Y¹ and Y² may be same or different and independently represent hydrogen, cyano, halogen, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkylcarbonylamino(C1-C20)alkyl, (C1-C20)alkylaminocarbonyl, amino(C1-C20)alkyl, mono(C1-C20)alkylamino, di(C1-C20)alkylamino, (C1-C20)alkoxy, or any one or two of Y¹ or Y² may represent substituted or unsubstituted —CH═NOH, carboxylic acid or its derivatives. The substituents on Y¹ and Y² may be selected from hydroxy, cyano, amino, (C1-C20)alkyl, (C1-C20)alkoxy, acyl, carboxylic acid or its esters; their pharmaceutically acceptable salts.

Pharmaceutically acceptable salts forming part of this invention include salts derived from inorganic bases such as Li, Na, K, Ca, Mg, Fe, Cu, Zn, Mn; salts of organic bases such as N,N′-diacetylethylenediamine, betaine, caffeine, 2-diethylaminoethanol, 2-dimethylaminoethanol, N-ethylmorpholine, N-ethylpiperidine, glucamine, glucosamine, hydrabamine, isopropylamine, methylglucamine, morpholine, piperazine, piperidine, procaine, purines, theobromine, triethylamine, trimethylamine, tripropylamine, tromethamine, diethanolamine, meglumine, ethylenediamine, N,N′-diphenylethylenediamine, N,N′-dibenzylethylenediamine, N-benzyl phenylethylamine, choline, choline hydroxide, dicyclohexylamine, metformin, benzylamine, phenylethylamine, dialkylamine, trialkylamine, thiamine, aminopyrimidine, aminopyridine, purine, spermidine, and the like; chiral bases like alkylphenylamine, glycinol, phenyl glycinol and the like, salts of natural amino acids such as glycine, alanine, valine, leucine, isoleucine, norleucine, tyrosine, cystine, cysteine, methionine, proline, hydroxy proline, histidine, ornithine, lysine, arginine, serine, threonine, phenylalanine; unnatural amino acids such as D-isomers or substituted amino acids; guanidine, substituted guanidine wherein the substituents are selected from nitro, amino, alkyl such as methyl, ethyl, propyl and the like; alkenyl such as ethenyl, propenyl, butenyl and the like; alkynyl such as ethynyl, propynyl and the like; ammnonium or substituted ammonium salts and aluminum salts. Salts may include acid addition salts where appropriate which are, sulphates, nitrates, phosphates, perchlorates, borates, halides, acetates, tartrates, maleates, citrates, succinates, palmoates, methanesulphonates, benzoates, salicylates, hydroxynaphthoates, benzenesulfonates, ascorbates, glycerophosphates, ketoglutarates and the like.

Particularly preferred compounds according to this invention are

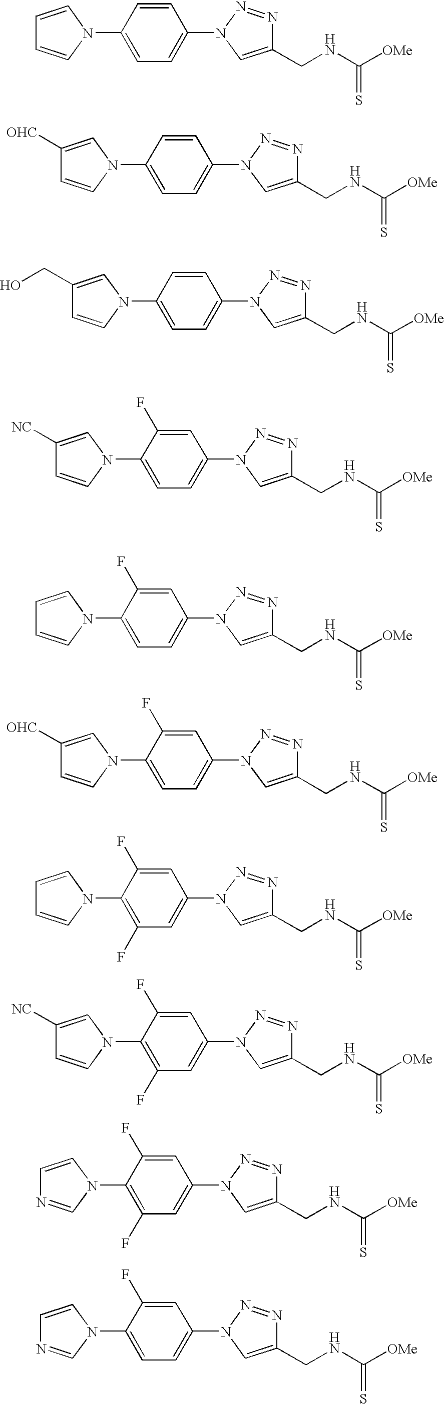

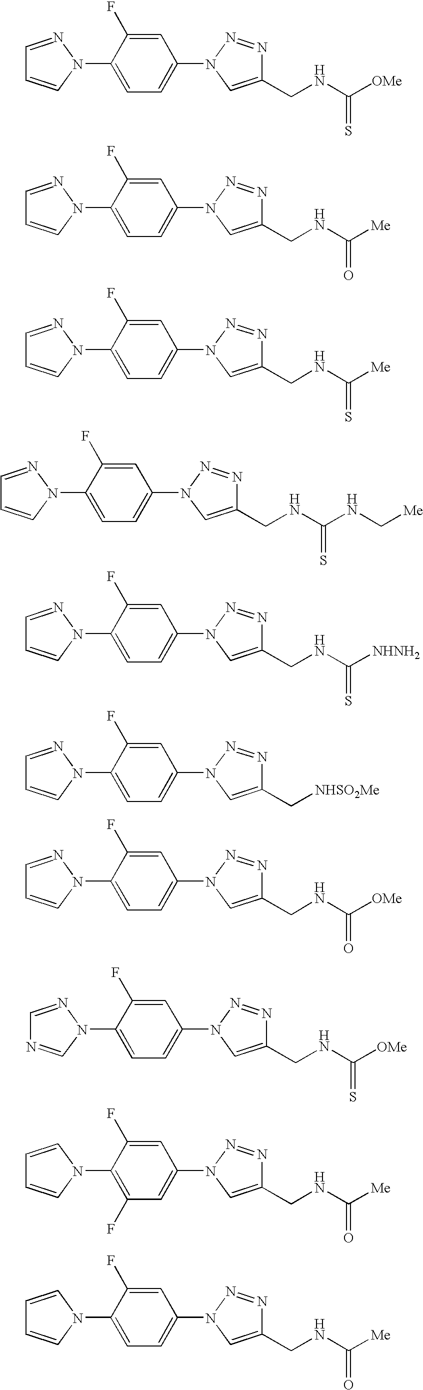

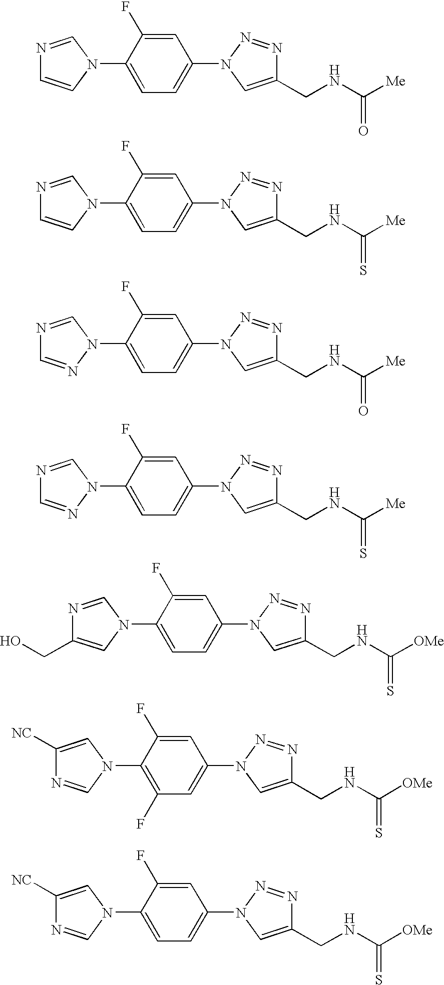

Another group of particulary useful compounds of the present invention are

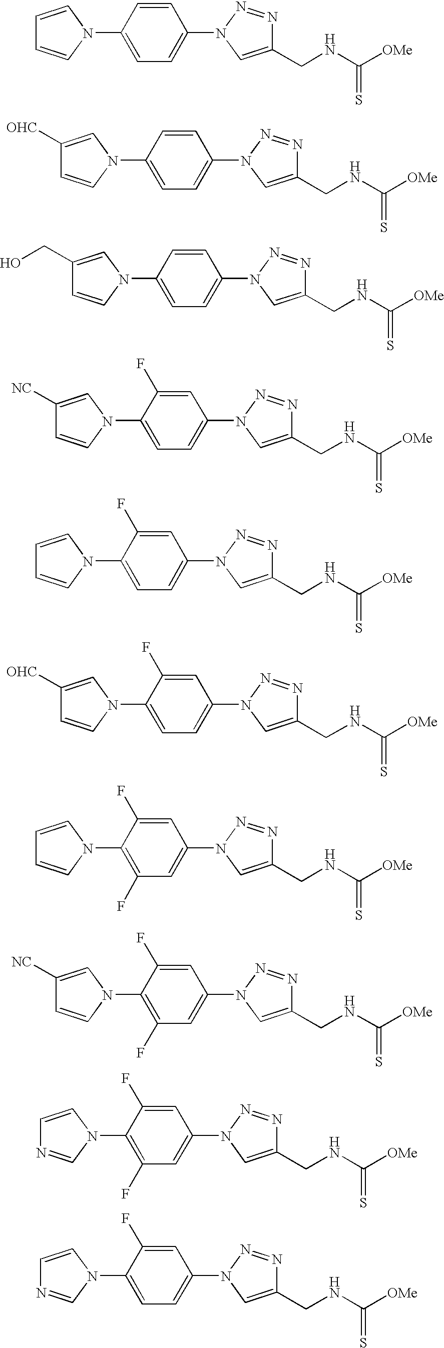

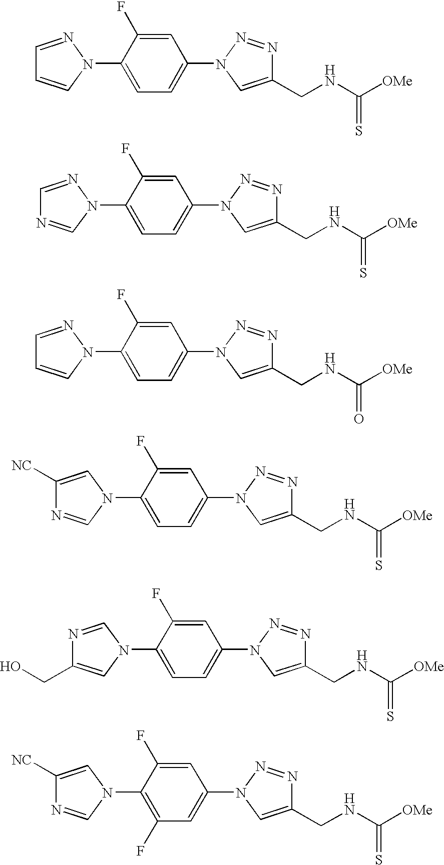

Another group of particulary useful compounds of the present invention are

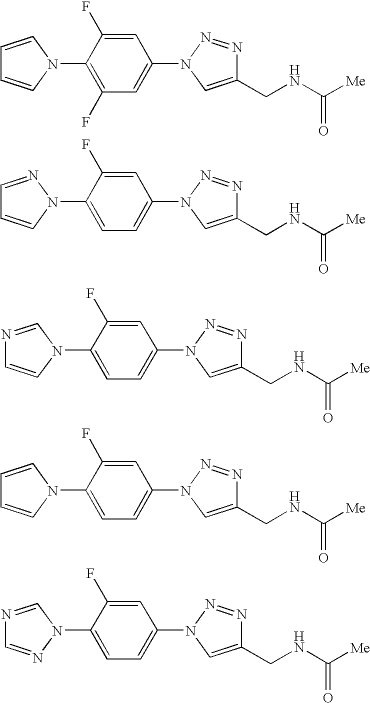

Another group of particulary useful compounds of the present invention are

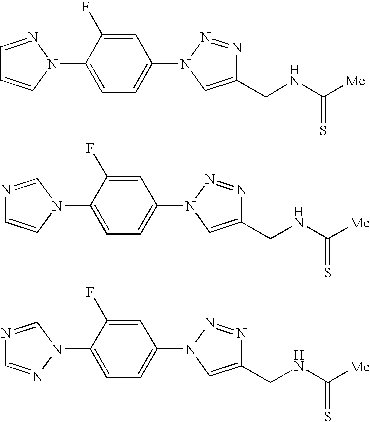

Yet another group of compounds of the present invention are

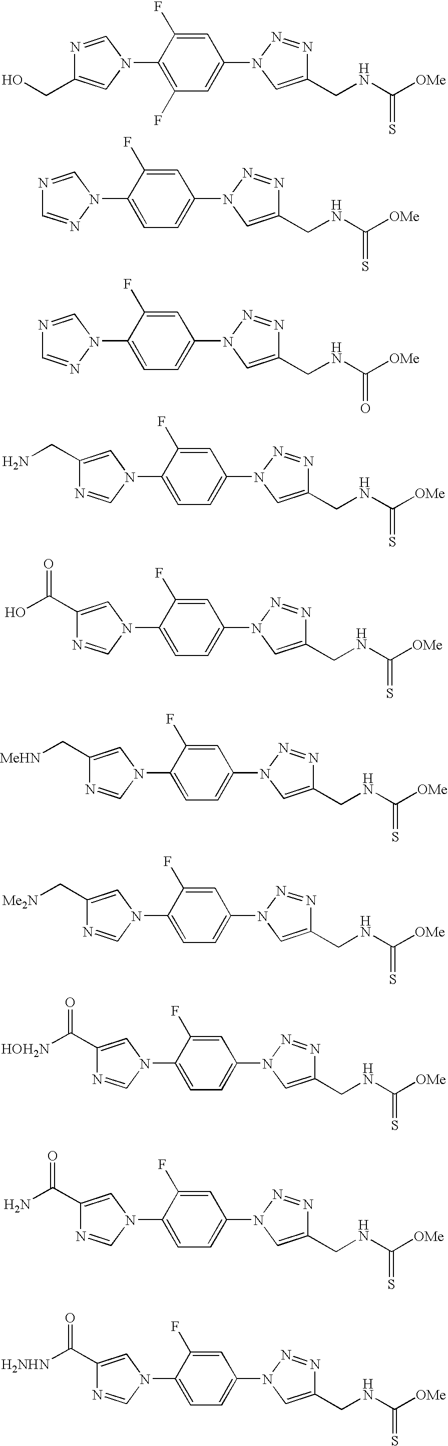

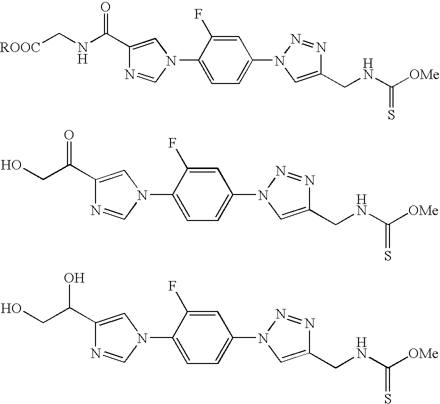

A preferred compound of the present invention is

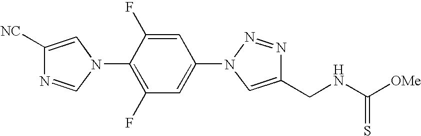

A preferred compound of the present invention is

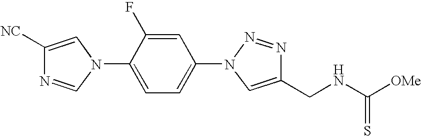

A preferred compound of the present invention is

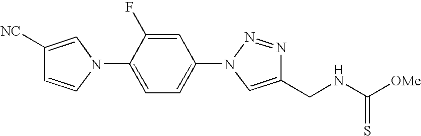

A preferred compound of the present invention is

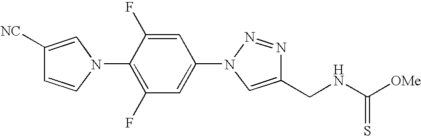

A preferred compound of the present invention is

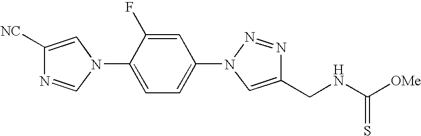

A preferred compound of the present invention is

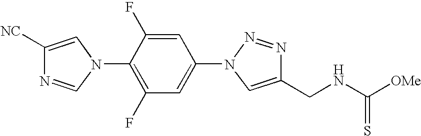

Yet another aspect of the present invention provides preparation of the the novel compounds of the present invention according to the procedure of the following schemes, using appropriate materials Those skilled in the art will readily understand that known variations of the conditions and processes of the following preparative procedures can be used to prepare these compounds. All temperatures are degrees Celsius unless otherwise noted.

The following Schemes describe procedures for making representative compounds of the present invention. Moreover, by utilizing the procedures described in detail, one of ordinary skill in the art can readily prepare additional compounds of the present invention claimed herein.

The process for the preparation of compounds of formula (I), where R¹ represents azido and all other symbols are as defined earlier, which comprises:

(i) reacting the compound of formula (Ia) 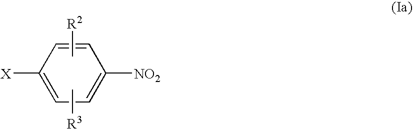
where X represents halogen atom such as fluorine, chlorine, bromine and the like; R² and R³ are as defined earlier, with a compound of formula (Ib) 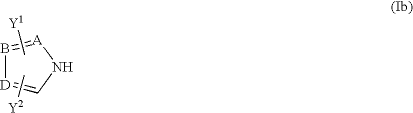
where A, B, D, Y¹ and Y² are as defined earlier, to produce a compound of formula (Ic) 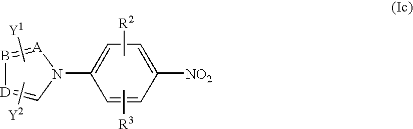
where A, B, D, Y¹, Y², R² and R³ are as defined earlier,

(ii) reducing the compund of formula (Ic) by using reducing agent to a compound of formula (Id) 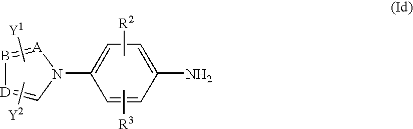
where A, B, D, Y¹, Y², R² and R³ are as defined earlier,

(iii) converting the compound of formula (Id) to a compound of formula (Ie) 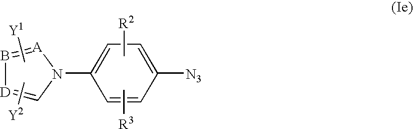
where A, B, D, Y¹, Y², R² and R³ are as defined earlier,

(iv) converting the compound of formula (Ie) to a compound of formula (If) 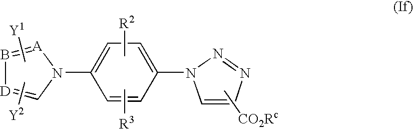
where R^{c} represents substituted or unsubstituted (C1-C20)alkyl group such as methyl, ethyl, n-propyl, iso-propyl and the like; A, B, D, Y¹, Y², R² and R³ are as defined earlier,

(v) reducing the compound of formula (If), to give a compound of formula (I) 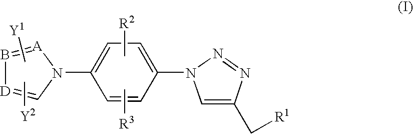
where R¹ represents hydroxy group; A, B, D, Y¹, Y², R² and R³ are as defined earlier,

(vi) converting the compound of formula (I), where R¹ represents hydroxy group, to a compound of formula (I), where R¹ represents substituted or unsubstituted (C1-C20)alkyl sulfonyloxy or arylsulfonyloxy group and all other symbols are as defined earlier, and

(vii) converting the compound of formula (I) where R¹ represents substituted or unsubstituted (C1-C20)alkylsulfonyloxy or arylsulfonyloxy group, to a compound of formula (I) where R¹ represents azido group and all other symbols are as defined earlier.

The compound of formula (Ic) may be prepared by reacting a compound of formula (Ia) with a compound of formula (Ib) by using a base such as potassium hydroxide (KOH), sodium hydroxide (NaOH), potassium carbonate (K₂CO₃), sodium carbonate (Na₂CO₃), sodium hydride (NaH), potassium hydride (KH), triethylamine, diisopropylethyl amine and the like. The reaction may be carried out using a solvent such as diemthyl sulfoxide (DMSO), dimethylformamide (DMF), tetrahydrofuran (THF), acetonitrile, chloroform, nitrobenzene and the like or mixtures thereof. The reaction may be carried out in inert atmosphere, which may be maintained using inert gases such as N₂ or Ar. The reaction may be carried out at a temperature in the range of 20 to 100° C., preferably at a temperature in the range of ambient—80° C. The reaction time may range from 1 to 15 h, preferably from 6 to 12 h.

The reduction of a compound of formula (Ic) to produce a compound of formula (Id) may be carried out in the presence of reducing agents such as NiCl₂/NaBH₄, lithium aluminium hydride (LAH), gaseous hydrogen and a catalyst such as Ru, Pd, Rh, Pt, Ni on solid beads such as charcoal, alumina, asbestos and the like. The reduction may be carried out in the presence of a solvent such as dioxane, acetic acid, ethyl acetate, THF, alcohol such as methanol, ethanol and the like or mixtures thereof. A pressure between atmospheric pressure to 60 psi may be used. The reaction may be carried out at a temperature from 0 to 60° C., preferably at 0 to room temperature. The reaction time ranges from 0.5 to 48 h, preferably in the range of 0.5 to 5 h. The reduction may also be carried out by employing metal in mineral acids such as Sn/HCl, Fe/HCl, Zn/HCl, Zn/CH₃CO₂H and the like.

The compound of formula (Id) may be converted to a compound of formula (Ie) by using sodium nitrite (NaNO₂) in the presence of HCl or acetic acid (CH₃COOH) followed by sodiumazide (NaN₃). The temperature of the reaction may be maintained in the range of −40° C. to boiling temperature, preferably in the range of 0° C. to room temperature. The duration of the reaction may be in the range of 0.5 to 15 h, preferably in the range of 0.5 to 5 h.

The compound of formula (If) may be prepared by heating a compound of formula (Ie) with (C₁-C₆)alkyl ester of propiolic acid. The solvent used in the reaction may be selected from benzene, toluene, xylene, acetonitrile, THF, DMF and the like. The temperature of the reaction may be maintained in the range of 10 to 200° C., preferably in the range of room temperature to the boiling temperature of the solvent. The duration of the reaction may be in the range of 1 to 25 h, preferably 5 to 20 h.

The conversion of compound of formula (If) to a compound of formula (I), where R¹ represents hydroxy may be carried out by using reducing agents such as LAH, lithiumboronhydride (LiBH₄) or sodium tetrahydroborate/iodine (NaBH₄/I₂). The reaction may be carried out in the presence of a solvent such as methanol, ethanol, THF, diethylether (Et₂O), dioxane and the like, or mixtures thereof. The temperature of the reaction may be in the range of −80 to 100° C., preferably 0° C. to boiling temperature of the solvent. The duration of the reaction may be in the range of 0.5 to 10 h.

The compound of formula (I) where R¹ represents OH may be converted to compound of formula (I) where R¹ represents substituted or unsubstituted (C1-C20)alkylsulfonyloxy or arylsulfonyloxy group, by treating with alkylsulfonylchloride or arylsulfonylchloride such as methanesulfonyl chloride, p-toluenesulfonyl chloride and the like. The reaction may be carried out in the presence of chloroform, dichloromethane, THF, dioxane and the like or mixtures thereof. The base used in the reaction may be selected from Et₃N, diisopropyl ethylamine, Na₂CO₃, K₂CO₃ and the like. The temperature of the reaction may be maintained in the range of 0 to 50° C., preferably in the range of 0 to room temperature. The duration of the reaction may be in the range of 1 to 12 h, preferably in the range of 1 to 4 h.

The compound of formula (I) where R¹ represents substituted or unsubstituted (C1-C20)alkyl sulfonyloxy or arylsulfonyloxy group may be converted to compound of formula (I) wherein R¹ represents azido group, by treating with NaN₃. The solvent used in the reaction may be selected from DMF, DMSO, acetonitrile, nitromethane and the like. The tempearature of the reaction may be maintained in the range of room temperature to 120° C., preferably room temperature to 80° C. The duration of the reaction may be in the range of 1 to 12 h, preferably 1 to 4 h.

Alternatively, the compound of formula (I) wherein R¹ represents hydroxy can be converted to a compound of formula (I) wherein R¹ represents azido group without isolating and characterizing the alkyl sulfonyl or arylsulfonyl intermediate formed.

Another embodiment of the present invention provides an alternative process for the preparation of the compound of formula (I) where R¹ represents azido and all other symbols are as defined earlier, which comprises:

(i) converting the compound of formula (Ie) 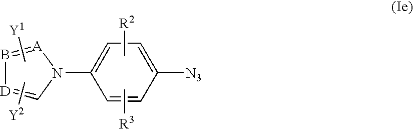
where A, B, D, Y¹, Y², R² and R³ are as defined earlier, to a compound of formula (I) 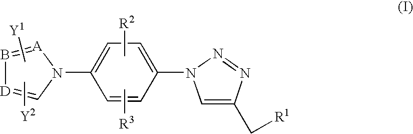
where R¹ represents hydroxy; A, B, D, Y¹, Y², R² and R³ are as defined earlier and

(ii) reacting the compound of formula (I) where R¹ represents hydroxy group, with MsCl, triethylamine and sodium azide to give a compound of formula (I) where R¹ represents azido group and all other symbols are as defined above

The compound of formula (Ie) may be converted to a compound of formula (I), where R¹ represents hydroxy group, by treating with propargyl alcohol. The solvent used in the reaction may be selected from benzene, toluene, xylene, acetonitrile, THF and the like. The temperature of the reaction may be maintained in the range of 10 to 200° C., preferably room temperature to the boiling temperature of the solvent. The duration of the reaction may be in the range of 1 to 25 h, preferably in the range of 5 to 20 h.

The compound of formula (I) where R¹ represents hydroxy group may be converted to a compound of formula (I) where R¹ represents azido group was carried out in two steps. In step (1) the compound of formula (I) where R¹ represents OH is converted to compound of formula (I) where R¹ represents leaving group such as halogen atom, by treating with CBr₄/PPh₃, PBr₃, thionylchloride (SOCl₂) and the like. The reaction may be carried out in the presence of chloroform, dichloromethane, THF, dioxane and the like or mixtures thereof. The temperature of the reaction may be maintained in the range of 0 to 80° C., preferably in the range of 0 to 50° C. The duration of the reaction may be in the range of 1-12 h, preferably in the range of 1-4 h. In step (2), the compound of formula (I) where R¹ represents halogen atom may be converted to compound of formula (I) where R¹ represents azido group by treating with NaN₃, LiN₃, trialkylsilylazide and the like. The solvent used in the reaction may be selected from acetone, THF, DMF, dimethyl sulfoxide (DMSO), acetonitrile and the like. The temperature of the reaction may be maintained in the range of room temperature to 120° C., preferably room temperature to 80° C. The duration of the reaction may be in the range of 1 to 12 h, preferably 1 to 4 h.

Yet another embodiment of the present invention provides an alternative process for the preparation of compound of formula (I), where R¹ represents azido group, which comprises:

(i) converting the compound of formula (Ie) 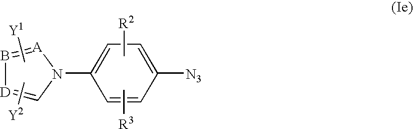
where A, B, D, Y¹, Y², R² and R³ are as defined earlier, to a compound of formula (I) 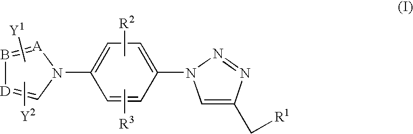
where R¹ represents halogen atom such as chlorine, bromine and the like, and all other symbols are as defined earlier and

(ii) converting the compound of formula (I) where R¹ represents halogen atom such as chlorine, bromine and the like, to a compound of formula (I), wherein R¹ represents azido group.

The compound of formula (I), where R¹ represents halogen atom such as chlorine, bromine and the like, may be prepared from a compound of formula (Ie) by using propargyl halide such as propargylchloride or propargyl bromide. The solvent used in the reaction may be selected from benzene, toluene, xylene, acetonitrile, THF and the like. The temperature of the reaction may be maintained in the range of 10 to 200° C., preferably room temperature to the boiling temperature of the solvent. The duration of the reaction may be in the range of 1 to 25 h, preferably in the range of 5 to 20 h.

The conversion of a compound of formula (I) where R¹ represents halogen atom such as chlorine, bromine and the like, to a compound of formula (I) where R¹ represents azido group, may be carried out in the presence of one or more equivalents of metal azide such as LiN₃, NaN₃ or trialkyl silylazide. The reaction may be carried out in the presence of solvent such as THF, acetone, DMF, DMSO and the like or mixtures thereof. The reaction may be carried out in inert atmosphere, which may be maintained using N₂ or Ar. The reaction may be carried out at a temperature in the range of ambient temperature to reflux temperature of the solvent, preferably at a temperature in the range of 50 to 80° C. The reaction time may be in the range from 0.5 to 18 h, preferably 1 to 4 h.

Another embodiment of the present invention provides a process for the preparation of compound of formula (I) where R¹ represents NHR⁴ wherein R⁴ represents hydrogen atom, which comprises:

(i) converting the compound of formula (If) 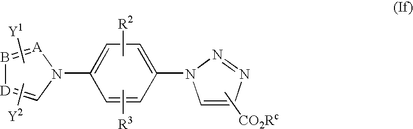
where R^{c} represents substituted or unsubstituted (C1-C20)alkyl group such as methyl, ethyl, n-propyl, iso-propyl and the like; A, B, D, Y¹, Y², R² and R³ are as defined earlier, to a compound of formula (Ig) 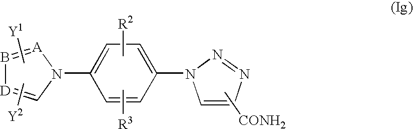
where all symbols are as defined earlier and

(ii) reducing the compound of formula (Ig), to produce a compound of formula (I)

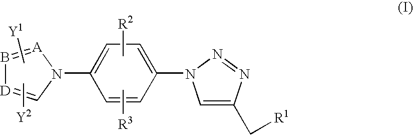

where R¹ represents NHR⁴ wherein R⁴ represents hydrogen atom and all other symbols are as defined earlier.

The conversion of compound of formula (If) to a compound of formula (Ig) may be carried out in the presence of ammonia solution in water or alcohol. The temperature of the reaction may be in the range of −40 to 50° C., preferably of 0° C. to room temperature. The duration of the reaction may be in the range of 0.5 to 12 h, preferably 0.5 to 4 h.

The reduction of compound of formula (Ig) to a compound of formula (I), where R¹ represents NHR⁴ wherein R⁴ represents hydrogen atom, may be carried out by using borane complex in THF, diethylether, SMe₂ or amine. The solvent used in the reaction may be selected from THF, diethylether, dioxane and the like. The temperature of the reaction may be in the range of −20 to 70° C., preferably 0 to boiling temperature of the solvent. The duration of the reaction may be in the range of 1 to 15 h, preferably 1 to 6 h.

Yet another embodiment of the present invention provides an alternative process for the preparation of compound of formula (I) where R¹ represents NHR⁴ wherein R⁴ represents hydrogen atom, which comprises:

(i) reducing the compound of formula (I) wherein R¹ represents azido group, to produce compound of formula (I) 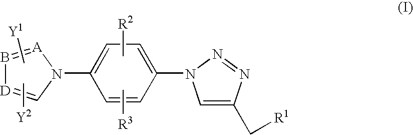
where R¹ represents NHR⁴ wherein R⁴ represents hydrogen atom; A, B, D, Y¹, Y², R² and R³ are as defined earlier.

The reduction of a compound of formula (I) where R¹ represents azido group, to produce a compound of formula (I) where R¹ represents NHR⁴ wherein R⁴ represents hydrogen atom, may be carried out in the presence of gaseous hydrogen and a catalyst such as Ru, Pd, Rh, Pt, Ni on solid beads such as charcoal, alumina, asbestos and the like. The reduction may be carried out in the presence of a solvent such as dioxane, acetic acid, ethyl acetate, THF, alcohol such as methanol, ethanol and the like or mixtures thereof. A pressure between atmospheric pressure to 60 psi may be used. The reaction may be carried out at a temperature in the range of 25 to 60° C., preferably at room temperature. The duration of the reaction may be in the range of 2 to 48 h. The reduction may also be carried out by employing PPh₃ in water.

Another embodiment of the present invention provides a process for the preparation of compound of formula (I) where R¹ represents hydroxy group, which comprises:

(i) converting the compound of formula (Ie), 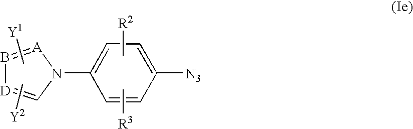
where A, B, D, Y¹, Y², R² and R³ are as defined earlier, to a compound of formula (I), 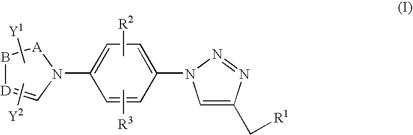
where R¹ represents substituted or unsubstituted (C1-C20)acyloxy group, and all other symbols are as defined earlier and

(ii) hydrolysis of the compound of formula (I) where R¹ represents (C1-C20)acyloxy group, to a compound of formula (I), where R¹ represents hydroxy group and all other symbols are as defined earlier.

The conversion of compound of formula (Ie) to a compound of formula (I) where R¹ represents (C1-C20)acyloxy group, may be carried out in the presence of esters ((C1-C20)alkyl or aryl)of propargyl alcohol. The solvent used in the reaction may be selected from benzene, toluene, xylene, acetonitrile, THF and the like. The temperature of the reaction may be maintained in the range of 10 to 200° C., preferably room temperature to the boiling temperature of the solvent. The duration of the reaction may be in the range of 1 to 25 h, preferably in the range of 5 to 20 h.

The hydrolysis of compound of formula (I) where R¹ represents (C1-C20)acyloxy, group, to a compound of formula (I), where R¹ represents hydroxy group, may be carried out by using conventional ester hydrolysis procedures.

The present invention also relates to a process for the preparation of the compound of formula (I) where R¹ represents azido and all other symbols are as defined earlier, which comprises:

(i) reacting the compound of formula (Ia) 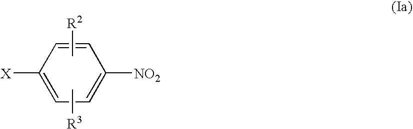
where X represents halogen atom such as fluorine, chlorine, bromine and the like; R² and R³ are as defined earlier, with a compound of formula (II) 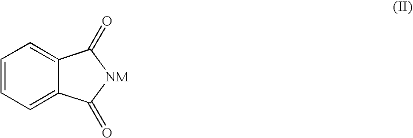
where M represents metal atom such as sodium, potassium and the like, to produce a compound of formula (2m) 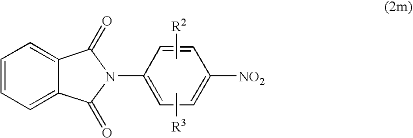
where R² and R³ are as defined earlier,

(ii) reducing the compound of formula (Im) by using reducing agent to a compound of formula (In) 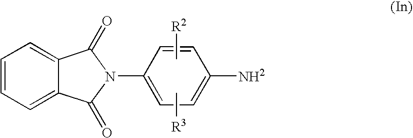
where R² and R³ are as defined earlier,

(iii) converting the compound of formula (In) to a compound of formula (Io) 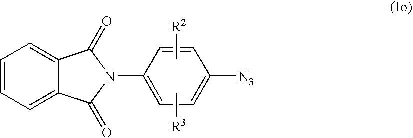
where R² and R³ are as defined earlier,

(iv) reacting the compound of formula (Io), with compound of formula (Ip) 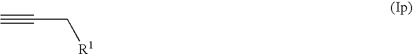
where R¹ is as defined in the description, to obtain a compound of formula (Iq) 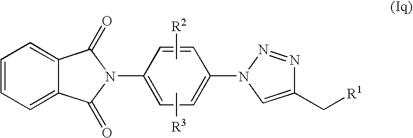
where R¹ represents NHR⁴ wherein R⁴ is as defined in the description, R² and R³ are as defined earlier,

(v) converting the compound of formula (Iq), to a compound of formula (Ir) 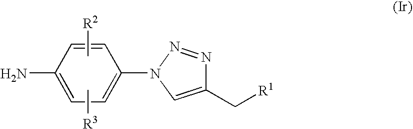
where R¹ is as defined above, R² and R³ are as defined earlier,

(vi) converting the compound of formula (Ir), to a compound of formula (I) 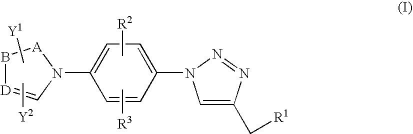
where R¹ is as defined above; R² and R³ are as defined earlier,

The compound of formula (Im) may be prepared by reacting a compound of formula (Ia) with a compound of formula (II). The reaction may be carried out using a solvent such as DMSO, DMF, THF, acetonitrile, chloroform, nitrobenzene and the like or mixtures thereof. The reaction may be carried out in inert atmosphere, which may be maintained using inert gases such as N₂ or argon (Ar). The reaction may be carried out at a temperature in the range of 20 to 100° C., preferably at a temperature in the range of ambient to 80° C. The reaction time may range from 1 to 15 h, preferably from 6 to 12 h.

The reduction of a compound of formula (Im) to produce a compound of formula (In) may be carried out in the presence of reducing agents such as NiCl₂/NaBH₄, lithium aluminium hydride (LAH), gaseous hydrogen and a catalyst such as Ru, Pd, Rh, Pt, Ni on solid beads such as charcoal, alumina, asbestos and the like. The reduction may be carried out in the presence of a solvent such as dioxane, acetic acid, ethyl acetate, THF, alcohol such as methanol, ethanol and the like or mixtures thereof. A pressure between atmospheric pressure to 60 psi may be used. The reaction may be carried out at a temperature from 0 to 60° C., preferably at 0 to room temperature. The reaction time ranges from 0.5 to 48 h, preferably in the range of 0.5 to 5 h. The reduction may also be carried out by employing metal in mineral acids such Sn/HCl, Fe/HCl, Zn/HCl, Zn/CH₃CO₂H and the like.

The compound of formula (In) may be converted to a compound of formula (Io) by using NaNO₂ in the presence of HCl or CH₃COOH followed by NaN₃. The temperature of the reaction may be maintained in the range of −40° C. to boiling temperature, preferably in the range of 0° C. to room temperature. The duration of the reaction may be in the range of 0.5 to 15 h, preferably in the range of 0.5 to 5 h.

Alternatively, the compound of formula (In) is converted to a compound of formula (Io) by using alkylnitrite such as t-butylnitrite and the like along with alkali metal azide such as sodium azide, potassium azide and the like. The solvent used in the reaction is selected from benzene, toluene, DMF, alcohol such as methanol, ethanol, propanol, isopropanol, butanol, tertiary butanol and the like. The temperature of the reaction may be maintained in the range of −40° C. to boiling temperature, preferably in the range of 0° C. to room temperature. The duration of the reaction may be in the range of 0.5 to 15 h, preferably in the range of 0.5 to 5 h.

The compound of formula (Io) is reacted with compound of formula (Ip), to obtain a compound of formula (Iq) by using Cu(I) halide in the presence or absence of a base such as DMAP, pyridine, triethylamine, diisopropylethylamine, lutidine and the like. The solvent used in the reaction may be selected from DMF, DMSO, THF, ether, dioxane, acetonitrile and the like.

The compound of formula (Iq) is converted to a compound of formula (Ir) by using hydrazine hydrate or an amine such as methylamine, ethylamine, ethylene diamine etc. The solvent used in the reaction is selected from methanol, ethanol, propanol, isopropanol and the like or mixtures thereof.

The compound of formula (Ir) is converted to a compound of formula (I), by treating with 2,5-dimethoxytetrahydrofuran or 2,5-dimethoxytetrahydrofuran-3-carboxaldehyde. The solvent used in the reaction is selected from acetic acid, propanoic acid and the like. The temperature of the reaction is maintained in the range of −20° C. to boiling temperature of the solvent used. The duration of the reaction is in the range of 0.5 to 15 h, preferably 0.5 to 10 h.

Another embodiment of the present invention provides a process for the preparation of compound of formula (I), where R¹ represents azido group, which comprises:

(i) converting the compound of formula (Io) 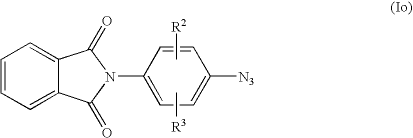
where R² and R³ are as defined earlier, to a compound of formula (Is) 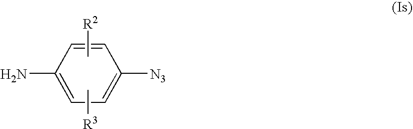
where R² and R³ are as defined earlier.

(ii) converting the compound of formula (Is), to a compound of formula (Ie) 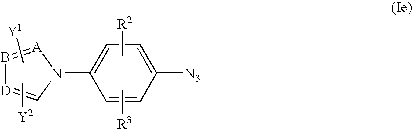
where all symbols are as defined earlier and

(iii) reacting the compound of formula (Ie), with a compound of formula (Ip) 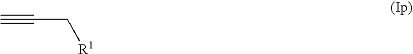
where R¹ is as defined in the description, to a compound of formula (I) 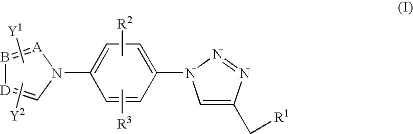
where R¹ is as defined in the description and all other symbols are as defined earlier

The compound of formula (Io) is converted to a compound of formula (Is) by using hydrazine hydrate or an amine such as methylamine, ethylamine, ethylene diamine etc. The solvent used in the reaction is selected from methanol, ethanol, propanol, isopropanol and the like or mixtures thereof.

The compound of formula (Is) is converted to a compound of formula (Ie), by treating with 2,5-dihydroxymethoxytetrahydrofuran or 2,5-dimethoxytetrahydro-3-carboxaldehyde. The solvent used in the reaction is selected from acetic acid, propanoic acid and the like. The temperature of the reaction is maintained in the range of −20° C. to boiling temperature of the solvent used. The duration of the reaction is in the range of 0.5 to 15 h, preferably 0.5 to 10 h.

The compound of formula (Ie) is reacted with compound of formula (Ip), to obtain a compound of formula (I) by using Cu(I) halide. The solvent used in the reaction may be selected from DMF, DMSO, THF, ether, dioxane, acetonitrile and the like.

Another embodiment of the present invention provides an alternate process for the preparation of compound of formula (Ir), which comprises:

(i) converting the compound of formula (Iu) 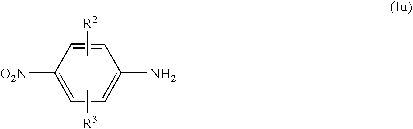
where R² and R³ are as defined earlier, to a compound of formula (Iv) 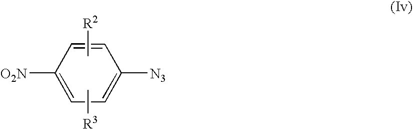
where R² and R³ are as defined earlier,

(ii) reacting the compound of formula (Iv), with a compound of formula (Ip) 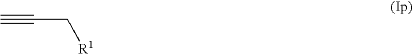
where R¹ is as defined in the description, to a compound of formula (Ir) 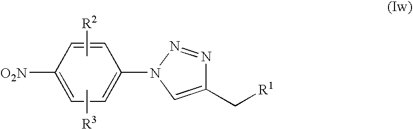
where R¹ is as defined in the description, R² and R³ are as defined earlier,

(iii) reducing the compound of formula (Iw), to a compound of formula (Ir) 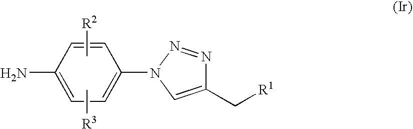
where all symbols are as defined above.

The compound of formula (Iu) may be converted to a compound of formula (Iv) by using NaNO₂ in the presence of HCl or CH₃COOH followed by NaN₃. The temperature of the reaction may be maintained in the range of −40° C. to boiling temperature, preferably in the range of 0° C. to room temperature. The duration of the reaction may be in the range of 0.5 to 15 h, preferably in the range of 0.5 to 5 h.

Alternatively, the compound of formula (Iu) is converted to a compound of formula (Iv) by using alkyl nitrite such as t-butylnitrite and the like along with alkali metal azide such as sodium azide, potassium azide and the like. The solvent used in the reaction is selected from benzene, toluene, DMF, alcohol such as methanol, ethanol, propanol, isopropanol, butanol, tertiary butanol and the like. The temperature of the reaction may be maintained in the range of −40° C. to boiling temperature, preferably in the range of 0° C. to room temperature. The duration of the reaction may be in the range of 0.5 to 15 h, preferably in the range of 0.5 to 5 h.

The compound of formula (Iv) is reacted with compound of formula (Ip), to obtain a compound of formula (Iw) by using Cu(I) halide in the presence or absence of a base such as 4-(dimethylamino)pyridine (DMAP), pyridine, triethylamine, diisopropylethylamine, lutidine and the like. The solvent used in the reaction may be selected from DMF, DMSO, THF, ether, dioxane, acetonitrile and the like.

The reduction of a compound of formula (Iw) to produce a compound of formula (Ir) may be carried out in the presence of reducing agent such as NiCl₂/NaBH₄, lithium aluminium hydride (LAH), gaseous hydrogen and a catalyst such as Ru, Pd, Rh, Pt, Ni on solid beads such as charcoal, alumina, asbestos and the like. The reduction may be carried out in the presence of a solvent such as dioxane, acetic acid, ethyl acetate, THF, alcohol such as methanol, ethanol and the like or mixtures thereof. A pressure between atmospheric pressure to 60 psi may be used. The reaction may be carried out at a temperature from 0 to 60° C., preferably at 0 to room temperature. The reaction time ranges from 0.5 to 48 h, preferably in the range of 0.5 to 5 h. The reduction may also be carried out by employing metal in mineral acids such Sn/HCl, Fe/HCl, Zn/HCl, Zn/CH₃CO₂H, Fe/NH₄Cl and the like.

Yet another embodiment of the present invention provides a process for the preparation of compound of formula (I), where R¹ represents NHR⁴, wherein R⁴ represents substituted or unsubstituted acetyl group and all other symbols are as defined earlier, from a compound of formula (I) where R¹ represents azido group, 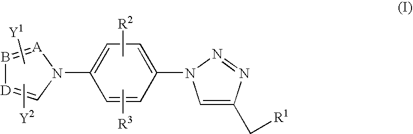
where A, B, D, Y¹, Y², R² and R³ are as defined earlier.

The compound of formula (I), where R¹ represents NHR⁴, wherein R⁴ represents substituted or unsubstituted acetyl group may be prepared from compound of formula (I), where R¹ represents azido group may be carried out by using thiolacetic acid with or without using solvent such as THF, DMF, toluene and the like. The reaction may be carried out at a temperature in the range of 25 to 40° C., preferably at room temperature. The duration of the reaction may be in the range from 3 to 24 h, preferably from 4 to 12 h.

Still another embodiment of the present invention provides a process for the preparation of compound of formula (I), where R¹ represents NHR⁴, where R⁴ represents substituted or unsubstituted —C(═S)—R^{4a}, wherein R^{4a} represents (C1-C20)alkyl, halo(C1-C20)alkyl, aryl, heteroaryl, —C(═O)—(C1-C20)alkoxy, —C(═O)—(C1-C20)alkoxy, —C(═O)-aryloxy, —C(═S)—(C1-C20)alkyl or —C(═S)-aryl; from compound of formula (I), where R¹ represents NHR⁴, where R⁴ represents substituted or unsubstituted —C(═O)—R^{4a}, wherein R^{4a} represents (C1-C20)alkyl, halo(C1-C20)alkyl, aryl, heteroaryl, —C(═O)—(C1-C20)alkoxy, —C(═O)-aryloxy, —C(═S)—(C1-C20)alkyl or —C(═S)-aryl 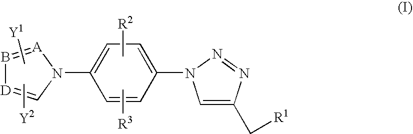
where all symbols are as defined earlier.

The compound of formula (I), where R¹ represents NHR⁴, wherein R⁴ represents substituted or unsubstituted —C(═S)—R^{4a}, from compound of formula (I), where R¹ represents NHR⁴, wherein R⁴ represents substituted or unsubstituted —C(═O)—R^{4a}, wherein R^{4a} is as defined above, may be carried out by taking a solution of the amide and Lawesson's reagent (2,4-bis(methoxyphenyl)-1,3-dithia-2,4-diphosphetane-2,4-disulfide) in dry dioxane, toluene, THF, DMF and the like. The reaction may be carried out at a temperature in the range of room temperature to 130° C., preferably in the range of 55 to 90° C. The duration of the reaction may be in the range from 3 to 24 h, preferably from 3 to 10 h.

Another embodiment of the present invention provides a process for the preparation of compound of formula (I), where R¹ represents NHR⁴, wherein R⁴ represents substituted or unsubstituted —C(═S)—OR^{4b}, wherein R^{4b} represents (C1-C20)alkyl, (C3-C20)cycloalkyl, aryl, (C2-C20)alkenyl or —C(═O)—(C1-C20)alkyl group, which comprises:

(i) reacting compound of formula (I) 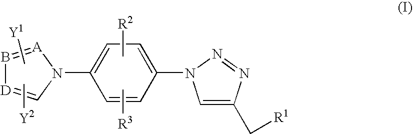
where R¹ represents azido group; and all other symbols are as defined earlier, with triphenylphosphine/water or H₂—Pd/C, to produce a compound of formula (I), where R¹ represents NHR⁴, wherein R⁴ represents hydrogen atom and all other symbols are as defined earlier,

(ii) reacting compound of formula (I), where R¹ represents NHR⁴, wherein R⁴ represents hydrogen atom, with thiophosgene or carbon disulfide and chloromethylformate, in the presence of a base to produce a compound of formula (I), where R¹ represents isothiocyanate group; and all symbols are as defined earlier,

(iii) converting compound of formula (I) where R¹ represents isothiocynate group, to a compound of formula (I), where R¹ represents NHR⁴, wherein R⁴ represents substituted or unsubstituted —C(═S)—OR^{4b}, wherein R^{4b} is as defined above and all other symbols are as defined earlier.

The conversion of compound of formula (I), where R¹ represents azido to a compound of formula (I), where R¹ represents NHR⁴, wherein R⁴ represents hydrogen atom may be carried out in the presence of gaseous hydrogen and a catalyst such as Ru, Pd, Rh, Pt, Ni on solid beads such as charcoal, alumina, asbestos and the like. The reduction may be conducted in the presence of a solvent such as dioxane, acetic acid, ethyl acetate, THF, alcohol such as methanol, ethanol, propanol, isopropanol and the like or mixtures thereof. A pressure between atmospheric pressure to 60 psi may be used. The reaction may be carried out at a temperature in the range of 25 to 60° C., preferably in the range of room temperature to 80° C. The duration of the reaction may be in the range of 2 to 48 h, preferably in the range of 5 to 15 h. The reduction may also be carried out by employing PPh₃ and water.

The compound of formula (I), where R¹ represents NHR⁴, wherein R⁴ represents hydrogen atom may be converted to a compound of formula (I) where R¹ represents isothiocyanate group, by using thiophosgene or carbon disulfide and chloromethylformate in the presence of a base such as Et₃N, K₂CO₃, NaOH and the like. The reaction may be carried out in the presence of a solvent such as dichloromethane (CH₂Cl₂), acetonitrile, chloroform (CHCl₃), DMF, THF and the like. The reaction may be carried at a temperature in the range of 0 to 60° C., preferably at 0° C. The reaction may be carried out in an inert atmosphere using argon or any other inert gas. The duration of the reaction may be in the range of 1 to 24 h, preferably 2 to 10 h.

The conversion of compound of formula (I) where R¹ represents isothiocyanate group, to a compound of formula (I), where R¹ represents NHR⁴, wherein R⁴ represents substituted or unsubstituted —C(═S)—OR^{4b}, wherein R^{4b} is as defined above, may be carried out by using respective alcohol such as methanol, ethanol, propanol, cyclohexanol and the like, in the absence or presence of a base such as NaH, KH and the like. The reaction may be carried out in the presence of a solvent such as THF, toluene, DMF and the like. The reaction may be carried out at a temperature in the range of room temperature to 130° C., preferably at reflux temperature of the solvent used. The duration of the reaction may be in the range of 6 to 24 h.

Another embodiment of the present invention provides a process for the preparation of compound of formula (I), where R¹ represents NHR⁴, where R⁴ represents substituted or unsubstituted groups selected from —C(═S)—NH₂, —C(═S)—NH—(C1-C20)alkyl, —C(═S)—N—((C1-C20)alkyl)₂, —C(═S)—NH—(C2-C20)alkenyl, C(═S)—NH—C(═O)-aryl, —C(═S)—NH-aralkyl, —C(═S)—NH-heteroaralkyl or —C(═S)—N(R′R″), wherein R′ and R″ groups together form a substituted or unsubstituted 5 or 6 membered cyclic structures containing nitrogen and optionally one or two additional hetero atoms selected from oxygen, nitrogen or sulfur; from a compound of formula (I) where R¹ represents isothiocyanate group, 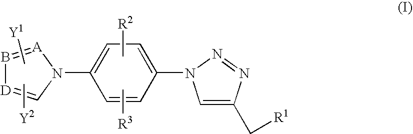
where all symbols are as defined earlier.

The compound of formula (I), where R¹ represents NHR⁴, wherein R⁴ represents substituted or unsubstituted —C(═S)—NH₂, may be prepared by passing ammonia gas into a solution of compound of formula (I) where R¹ represents isothiocyanate group, in the presence of a solvent such as THF, toluene, and the like. The reaction may be carried out at a temperature in the range of −10° C. to room temperature, preferably at +10° C. The duration of the reaction may be in the range from 20 min to 4 h, preferably 30 min.

The compound of formula (I), where R¹ represents NHR⁴, wherein R⁴ represents substituted or unsubstituted groups selected from —C(═S)—NH—(C1-C20)alkyl, —C(═S)—N—((C1-C20)alkyl)₂, —C(═S)—NH—(C2-C20)alkenyl, C(═S)—NH—C(═O)-aryl, —C(═S)—NH-aralkyl, —C(═S)—NH-heteroaralkyl or —C(═S)—N(R′R″), wherein R′ and R″ groups together form a substituted or unsubstituted 5 or 6 membered cyclic structures containing nitrogen and optionally one or two additional hetero atoms selected from oxygen, nitrogen or sulfur, may be carried out by treating a compound of formula (I) where R¹ represents isothiocyanate group with appropriate amine such as methylamine, ethylamine, diemthylamine, diethylamine, benzylamine, aniline, proline, morpholine, thiomorpholine, pyridiylmethylamine and the like, in the presence of a solvent such as THF, DMF, toluene, and the like. The reaction may be carried out at a temperature in the range of room temperature to 140° C., preferably at room temperature to 100° C. The duration of the reaction may be in the range of 0.5 to 24 h, preferably 0.5 to 12 h.

Yet another embodiment of the present invention provides a process for the preparation of compound of formula (I), where R¹ represents NHR⁴, wherein R⁴ represents substituted or unsubstituted —C(═S)—SR^{4c}, wherein R^{4c} represents (C1-C20)alkyl group, from compound of formula (I), where R¹ represents NHR⁴, wherein R⁴ represents hydrogen atom, 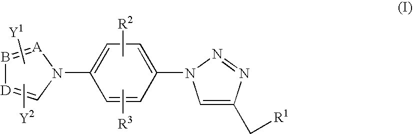
where all other symbols are as defined earlier.

The compound of fomula (I), where R¹ represents NHR⁴, wherein R⁴ represents substituted or unsubstituted —C(═S)—SR^{4c}, wherein R^{4c} is as defined above, may be prepared from compound of formula (I), where R¹ represents NHR⁴, wherein R⁴ represents hydrogen atom, by using CS₂ in the presence of a base such as Et₃N, diisopropyl ethylamine, K₂CO₃, NaH, t-BuOK and the like. The reaction may be carried out in the presence of alkyl halide such as methyliodide, ethylbromide, propylbromide and the like. The solvent used in the reaction may be selected from ethanol, methanol, isopropanol, THF, diethylether, acetonitrile and the like, or mixtures thereof. The reaction may be carried out at a temperature in the range of room temperature to 60° C., preferably at room temperature. The duration of the reaction may be in the range of 6 to 24 h.

Another embodiment of the present invention provides a process for the preparation of compound of formula (I), where R¹ represents NHR⁴, wherein R⁴ represents substituted or unsubstituted —C(═S)—NH—R^{4d}, wherein R^{4d} represents —C(═O)-aryl group, from compound of formula (I), where R¹ represents NHR⁴, wherein R⁴ represents hydrogen atom, 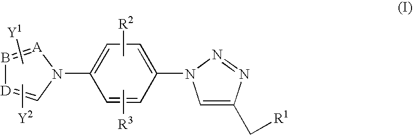
where all other symbols are as defined earlier.

The compound of fomula (I), where R¹ represents NHR⁴, wherein R⁴ represents substituted or unsubstituted —C(═S)—NH—R^{4d} wherein R^{4d} is as defined above, may be prepared from compound of formula (I), where where R¹ represents NHR⁴, wherein R⁴ represents hydrogen atom by using benzoylisothiocyanate. The solvent used in the reaction may be selected from acetone, ethanol, methanol, isopropanol, THF, diethylether, acetonitrile and the like. The temperature of the reaction may be maintained in the range of 0 to 80° C., preferably in the range of room temperature to 60° C. The duration of the reaction may be in the range of 1 to 20 h, preferably in the range of 1 to 10 h.

Yet another embodiment of the present invention provides a process for the preparation of compound of formula (I), where R¹ represents NHR⁴, wherein R⁴ represents substituted or unsubstituted —C(═O)-heteroaryl, from a compound of formula (I), where R¹ represents NHR⁴, wherein R⁴ represents hydrogen atom, 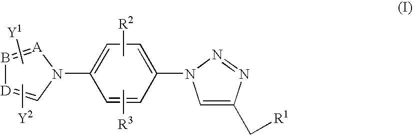
where all other symbols are as defined earlier.

The compound of fomula (I), where R¹ represents NHR⁴, wherein R⁴ represents substituted or unsubstituted —C(═O)-heteroaryl, may be prepared from compound of formula (I), where R¹ represents NHR⁴, wherein R⁴ represents hydrogen atom by treating with corresponding heteroaroyl acid chloride and base such such as pyridine, triethylamine or diisopropylamine. The reaction may also be carried out by using corresponding heteroaryl acid and dicyclohexylcarbodiimide (DCC) in the presence of DMAP. The solvent used in the reaction may be selected from acetonitrile, THF, acetonitrile, Et₂O and the like. The temperature of the reaction may be maintained in the range of −5 to 100° C., preferably in the range of 0 to 80° C. The duration of the reaction may be in the range of 1 to 15 h, preferably in the range of 2 to 12 h.

Still another embodiment of the present invention provides a process for the preparation of compound of formula (I), where R¹ represents NHR⁴ where R⁴ represents substituted or unsubstituted —C(═O)—R^{4e} wherein R^{4e} represents (C1-C20)alkyl, (C1-C20)alkoxy, (C2-C20)alkenyl, halo(C1-C20)alkyl, aryl, aryloxy, heteroaryl, (C2-C20)alkenyloxy, (C1-C20)alkylcarbonyl, arylcarbonyl, aryloxycarbonyl, (C1-C20)alkoxycarbonyl, (C1-C20)alkylthiocarbonyl or (C1-C20)arylthiocarbonyl; from a compound of formula (I), where R¹ represents NHR⁴, wherein R⁴ represents hydrogen atom, 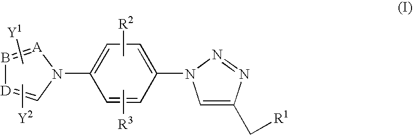
where all other symbols are as defined earlier.

The compound of formula (I), where R¹ represents NHR⁴, wherein R⁴ represents substituted or unsubstituted —C(═O)—R^{4e}, wherein R^{4e} is as defined above, may be prepared from compound of formula (I), where R¹ represents NHR⁴, wherein R⁴ represents hydrogen atom, by treating with appropriate acid halide such as acetyl chloride, propionyl chloride and the like; alkylchloroformate like methylchloroformate, ethylchloroformate and the like; aralkylchloroformate like benzylchloroformate and the like; or anhydride of the corresponding acid such as acetic anhydride. The reaction may be carried out in the presence of a solvent such as CH₂Cl₂, CHCl₃, toluene, THF and the like or mixtures thereof. The reaction may also be carried out in the presence of a base like Et₃N, diisopropyl ethylamine, pyridine, K₂CO₃, NaH, potassium tert-butoxide (t-BuOK) and the like. The temperature of the reaction may be maintained in the range of −20 to 60° C., preferably in the range of 0 to room temperature. The duration of the reaction may be in the range of 1 to 12 h, preferably from 1 to 4 h.

Yet another embodiment of the present invention provides a process for the preparation of compound of formula (I) where R¹ represents NHR⁴ where R⁴ represents substituted or unsubstituted —C(═NH)—NH₂, by reacting a compound of formula (I), where R¹ represents NHR⁴ wherein R⁴ represents hydrogen atom, with di-tert-butoxy carbonyl thiourea, 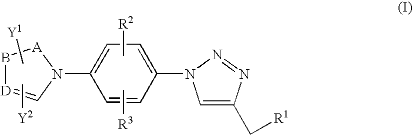
where all other symbols are as defined earlier.

The compound of formula (I) where R¹ represents NHR⁴ where R⁴ represents substituted or unsubstituted group selected from —C(═NH)—NH₂, may be prepared by reacting the compound of formula (I), where R¹ represents NHR⁴ where R⁴ represents hydrogen atom, with di-tert-butoxy carbonyl thiourea in two steps. In the first step, the reaction may be carried out in the presence of solvents such as DMF, acetone, THF, dichloromethane and the like. The base used in the reaction may be selected from triethylamine, diisopropylethylamine, pyridine and the like. The temperature of the reaction may be in the range of 0 to 120° C., preferably in the range of 0 to 90° C. The duration of the reaction may be in the range of 0.2 to 15 h, preferably in the range of 0.5 to 10 h. In the second step, the compound obtained in the first step may be reacted with trifluoroacetic acid in the presence of a solvent such as dichloromethane, chloroform, THF and the like. The temperature of the reaction may be in the range of 0 to 110° C., preferably in the range of 0 to 90° C. The duration of the reaction may be in the range of 0.5 to 60 h, preferably in the range of 0.5 to 54 h.

Another embodiment of the present invention provides an alternative process for the preparation of compound of formula (I) where R¹ represents NHR⁴ where R⁴ represents substituted or unsubstituted group selected from —C(═NH)—NH₂, by reacting a compound of formula (I), where R¹ represents NHR⁴ wherein R⁴ represents substituted or unsubstituted group selected from —S(O)₂(C1-C20)alkyl or —S(O)₂aryl group, with guanidine hydrochloride, 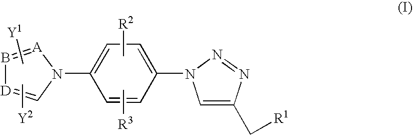
where all other symbols are as defined earlier.

The compound of formula (I) where R¹ represents NHR⁴ where R⁴ represents substituted or unsubstituted group selected from —C(═NH)—NH₂, may be prepared by reacting the compound of formula (I), where R¹ represents NHR⁴ wherein R⁴ represents substituted or unsubstituted group selected from —S(O)₂(C1-C20)alkyl or —S(O)₂aryl group, with guanidine hydrochloride. The solvent used in the reaction may be seleceted from t-butyl alcohol. The base used in the reaction may be selected from NaH, KH, sodium hexamethyldisilazide (Na-HMDS) and the like. The temperature of the reaction may be in the range of 0° C. to boiling temperature of the solvent used. The duration of the reaction may be in the range of 1 to 30 h, preferably in the range of 1 to 24 h.

Still another embodiment of the present invention provides a process for the preparation of compound of formula (I) where R¹ represents NHR⁴ where R⁴ represents substituted or unsubstituted group selected from —C(═NH)—(C1-C20)alkyl or —C(═NH)-aryl, which comprises:

(i) reacting the compound of formula (I) 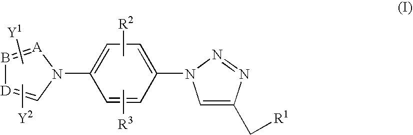
where R¹ repersents NHR⁴, wherein R⁴ represents —C(═S)—NH₂ and all other symbols are as defined earlier, with di tert-butoxy carbonyl ether ((BOC)₂O), to produce a compound of formula (I), where R¹ represents NHR⁴, wherein R⁴ represents —C(═S)—NH₂ group substituted with tert-butoxy carbonyl group and all symbols are as defined earlier and

(ii) reacting the above compound of formula (I), with a compound of formula (Ih)
R⁷—NH₂ (Ih)
where R⁷ represents substituted or unsubstituted (C1-C20)alkyl or aryl group, to produce a compound of formula (I) where R¹ represents NHR⁴ where R represents substituted or unsubstituted group selected from —C(═NH)—(C1-C20)alkyl or —C(═NH)-aryl group and all other symbols are as defined earlier.

The conversion of the compound of formula (I) where R¹ represents NHR⁴, wherein R⁴ represents —C(═S)—NH₂, to a compound of formula (I), where R¹ represents NHR⁴, wherein R⁴ represents —C(═S)—NH₂ group substituted with tert-butoxy carbonyl group may be carried out by reacting with (BOC)₂O, in the presence of solvent such as THF, diethylether and the like. The base used in the reaction may be selected from NaH, KH, Na-HMDS and the like. The temperature of the reaction may be in the range of 0 to boiling temperature of the solvent. The duration of the reaction may be in the range of 0.5 to 14 h, preferably in the range of 0.5 to 10 h.

The conversion of the compound of formula (I), where R¹ represents NHR⁴, wherein R⁴ represents —C(═S)—NH₂ group substituted with tert-butoxy carbonyl group, to a compound of formula (I) may be carried out by reacting with the compound of formula (Ih) in two steps. In the first step, the reaction may be carried out in the presence of a solvent such as DMF, THF, chloroform, dichloromethane and the like. The base used in the reaction may be selected from triethylamine, diisopropylethylamine, pyridine and the like. The temperature of the reaction may be in the range of 0 to 120° C., preferably in the range of 0 to 90° C. The duration of the reaction may be in the range of 0.5 to 24 h, preferably in the range of 0.5 to 20 h. In the second step, the compound obtained in the first step may be reacted with trifluoroacetic acid in the presence of a solvent such as dichloromethane, chloroform, THF and the like. The temperature of the reaction may be in the range of 0 to 110° C., preferably in the range of 0 to 90° C. The duration of the reaction may be in the range of 0.5 to 60 h, preferably in the range of 0.5 to 54 h.

Yet another embodiment of the present invention provides a process for the preparation of a compound of formula (I) where R¹ represents halogen, from compound of formula (I) where R¹ represents hydroxy group, 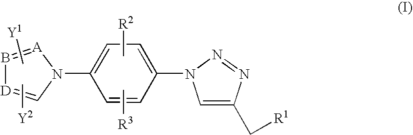
where all other symbols are as defined above.

The compound of formula (I) where R¹ represents halogen is prepared from compound of formula (I) where R¹ represents hydroxy group may be carried out by treating with SOCl₂, PCl₅, PBr₃, tetrahalomethane group such as CBr₄, CCl₄ and the like, in the presence of PPh₃, P(alkyl)₃ and the like. The reaction may be carried out in the presence of a solvent such as dry dichloromethane, chloroform, tetrachloromethane, benzene, DMF, DMSO, THF and the like. The temperature of the reaction may be maintained in the range of 0 to 60° C., preferably at room temperature. The duration of the reaction may be in the range of 0.5 to 24 hours, preferably 1 to 13 h.

Still another embodiment of the present invention provides a process for the preparation of a compound of formula (I) where R¹ represents ‘SH’, 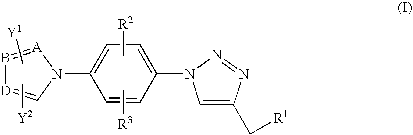
where all other symbols are as defined above, which comprises:

(i) reacting the compound of formula (I) where R¹ represents halogen atom, to produce a compound of formula (Ii), 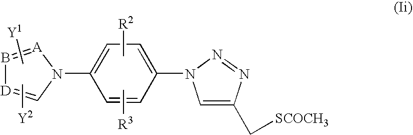
where all other symbols are as defined earlier, with a base and thiolacetic acid,

(ii) reacting the compound of formula (Ii), to produce a compound of formula (I) where R¹ represents ‘SH’ group and all other symbols are as defined earlier, with base.

The compound of formula (Ii) is prepared from compound of formula (I) where R¹ represents halogen atom may be prepared by using thiolacetic acid in the presence of a base such as triethylamine, di-isopropylamine, di-isopropylethylamine, pyridine, piperidine, DMAP, 1,8-diazabicyclo[5.4.0]undec-7-ene (DBU), lithium diisopropylamide (LDA), potassium bis-(trimethyl silyl)amide, BuLi, Na₂CO₃, K₂CO₃, NaOH, KOH, NaOMe, NaOEt, NaOiPr, t-BuOK, NaH, KH and the like. The solvent used in the reaction may be seleceted from THF, benzene, dioxane and the like. The temperature of the reaction may be maintained in the range of room temperature to reflux temperature, preferably at reflux temperature. The duration of the reaction may be in the range of 2 to 24 hours, preferably 6 hours.

The compound of formula (I), where R¹ represents ‘SH’ group may be prepared from a compound of formula (Ii) by reacting with a base such as K₂CO₃, NaOH, KOH, BuLi and the like. The reaction may be carried out at a temperature in the range of room temperature to reflux temprature. The duration of the reaction may be in the range of 1 to 24 hours.

Still yet another embodiment of the present invention provides a process for the preparation of compound of formula (I), where R¹ represent NHR⁴ wherein R⁴ represents substituted or unsubstituted —S(O)₂(C1-C20)alkyl or —S(O)₂aryl group, from a compound of formula (I) where R¹ represents NHR⁴ where R⁴ represents hydrogen atom,

(i) reacting the compound of formula (I), 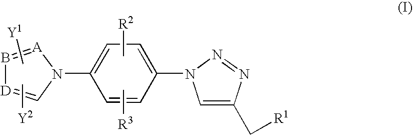
where R¹ represents NHR⁴ where R⁴ represents hydrogen atom and all other symbols are as defined in the description, to a compound of formula (I), where R¹ represents NHR⁴, wherein R⁴ represents substituted or unsubstituted group selected from —S(O)₂—(C1-C20)alkyl or —S(O)₂-aryl group and all other symbols are as defined in the description, to a compound of formula (I).

The conversion of compound of formula (I), where R¹ represents NHR⁴ where R⁴ represents hydrogen atom, to a compound of formula (I), where R¹ represents NHR⁴, wherein R⁴ represents substituted or unsubstituted group selected from —S(O)₂—(C1-C20)alkyl or —S(O)₂-aryl group, may be carried out by treating with alkylsulfonylchloride or arylsulfonylchloride such as methanesulfonyl chloride, p-toluenesulfonyl chloride and the like. The solvent used may be selected from dichloromethane, tetrahydrofuran, acetonitrile, dimethylformamide, dimethylsulfoxide and the like. The temperature of the reaction may be in the range of 0 to 50° C., for a duration of 1 to 6 hours.

Another embodiment of the present invention provides a novel intermediate of the formula (Ie), 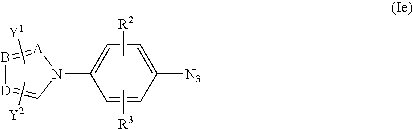
where A, B, D, Y¹, Y², R² and R³ are as defined earlier.

Yet another embodiment of the present invention provides a process for the preparation of novel intermediate of formula (Ie), which comprises:

(i) reacting the compound of formula (Ia) 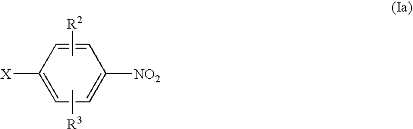
where X represents halogen atom such as fluorine, chlorine, bromine and the like; R² and R³ are as defined earlier, with a compound of formula (Ib) 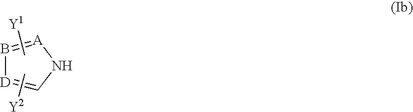
where A, B, D, Y¹ and Y² are as defined earlier, to produce a compound of formula (Ic) 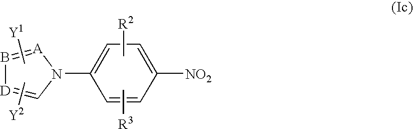
where A, B, D, Y¹, Y², R² and R³ are as defined earlier,

(ii) reducing the compund of formula (Ic) by using reducing agent to a compound of formula (Id) 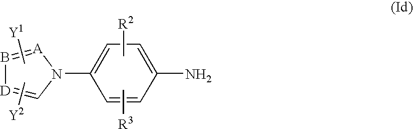
where A, B, D, Y¹, Y², R² and R³ are as defined earlier

(iii) converting the compound of formula (Id) to a compound of formula (Ie) 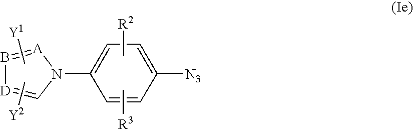
where A, B, D, Y¹, Y², R² and R³ are as defined earlier.

The compound of formula (Ic) may be prepared by reacting a compound of formula (Ia) with a compound of formula (Ib) by using a base such as KOH, NaOH, K₂CO₃, Na₂CO₃, NaH, KH, triethylamine, diisopropylethyl amine and the like. The reaction may be carried out using a solvent such as DMSO, DMF, THF, acetonitrile, chloroform, nitrobenzene and the like or mixtures thereof. The reaction may be carried out in inert atmosphere, which may be maintained using inert gases such as N₂ or Ar. The reaction may be carried out at a temperature in the range of 20 to 100° C., preferably at a temperature in the range of ambient −80° C. The reaction time may range from 1 to 15 hours, preferably from 6 to 12 hours.

The reduction of a compound of formula (Ic) to produce a compound of formula (Id) may be carried out in the presence of reducing agents such as NiCl₂/NaBH₄, lithium aluminium hydride (LAH), gaseous hydrogen and a catalyst such as Ru, Pd, Rh, Pt, Ni on solid beads such as charcoal, alumina, asbestos and the like. The reduction may be carried out in the presence of a solvent such as dioxane, acetic acid, ethyl acetate, THF, alcohol such as methanol, ethanol and the like or mixtures thereof. A pressure between atmospheric pressure to 60 psi may be used. The reaction may be carried out at a temperature from 0 to 60° C., preferably at 0 to room temperature. The reaction time ranges from 0.5 to 48 hours, preferably in the range of 0.5 to 5 hours. The reduction may also be carried out by employing metal in mineral acids such Sn/HCl, Fe/HCl, Zn/HCl, Zn/CH₃CO₂H and the like.

The compound of formula (Id) may be converted to a compound of formula (Ie) by using NaNO₂ in the presence of HCl or CH₃COOH followed by NaN₃. The solvent used in the reaction may be selected from methanol, ethanol, ethylacetate, THF, ether, dioxan and the like. The temperature of the reaction may be maintained in the range of −40° C. to boiling temperature, preferably in the range of 0° C. to room temperature. The duration of the reaction may be in the range of 0.5 to 15 hours, preferably in the range of 0.5 to 5 hours.

Another embodiment of the present invention provides a novel intermediate of the formula (If), 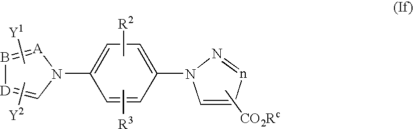
where R^{c} represents substituted or unsubstituted (C1-C20)alkyl group such as methyl, ethyl, n-propyl, iso-propyl and the like; A, B, D, Y¹, Y², R² and R³ are as defined earlier.

Yet another embodiment of the present invention provides a process for the preparation of novel intermediate of formula (If), which comprises:

(i) reacting the compound of formula (Ia) 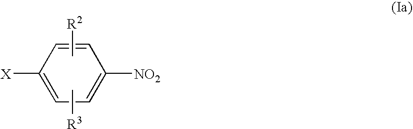
where X represents halogen atom such as fluorine, chlorine, bromine and the like; R² and R³ are as defined earlier, with a compound of formula (Ib) 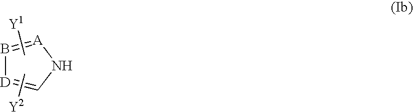
where A, B, D, Y¹ and Y² are as defined earlier, to produce a compound of formula (Ic) 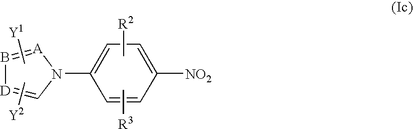
where A, B, D, Y¹, Y², R² and R³ are as defined earlier,

(ii) reducing the compund of formula (Ic) by using reducing agent to a compound of formula (Id) 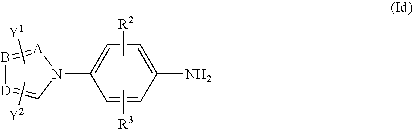
where A, B, D, Y¹, Y², R² and R³ are as defined earlier

(iii) converting the compound of formula (Id) to a compound of formula (Ie) 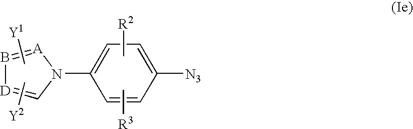
where A, B, D, Y¹, Y², R² and R³ are as defined earlier

(iv) converting the compound of formula (Ie) to a compound of formula (If) 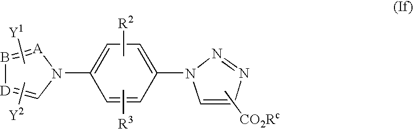
where R^{c} represents substituted or unsubstituted (C1-C20)alkyl group such as methyl, ethyl, n-propyl, iso-propyl and the like; A, B, D, Y¹, Y² , R² and R³ are as defined earlier.

The compound of formula (Ic) may be prepared by reacting a compound of formula (Ia) with a compound of formula (Ib) by using a base such as KOH, NaOH, K₂CO₃, Na₂CO₃, NaH, KH, triethylamine, diisopropylethyl amine and the like. The reaction may be carried out using a solvent such as DMSO, DMF, THF, acetonitrile, chloroform, nitrobenzene and the like or mixtures thereof. The reaction may be carried out in inert atmosphere, which may be maintained using inert gases such as N₂ or Ar. The reaction may be carried out at a temperature in the range of 20 to 100° C., preferably at a temperature in the range of ambient −80° C. The reaction time may range from 1 to 15 hours, preferably from 6 to 12 hours.

The reduction of a compound of formula (Ic) to produce a compound of formula (Id) may be carried out in the presence of reducing agents such as NiCl₂/NaBH₄, lithium aluminium hydride (LAH), gaseous hydrogen and a catalyst such as Ru, Pd, Rh, Pt, Ni on solid beads such as charcoal, alumina, asbestos and the like. The reduction may be carried out in the presence of a solvent such as dioxane, acetic acid, ethyl acetate, THF, alcohol such as methanol, ethanol and the like or mixtures thereof. A pressure between atmospheric pressure to 60 psi may be used. The reaction may be carried out at a temperature from 0 to 60° C., preferably at 0 to room temperature. The reaction time ranges from 0.5 to 48 hours, preferably in the range of 0.5 to 5 hours. The reduction may also be carried out by employing metal in mineral acids such Sn/HCl, Fe/HCl, Zn/HCl, Zn/CH₃CO₂H and the like.

The compound of formula (Id) may be converted to a compound of formula (Ie) by using NaNO₂ in the presence of HCl or CH₃COOH followed by NaN₃. The solvent used in the reaction may be selected from methanol, ethanol, ethylacetate, THF, ether, dioxan and the like. The temperature of the reaction may be maintained in the range of −40° C. to boiling temperature, preferably in the range of 0° C. to room temperature. The duration of the reaction may be in the range of 0.5 to 15 hours, preferably in the range of 0.5 to 5 hours.

The compound of formula (If) may be prepared by heating a compound of formula (Ie) with esters ((C1-C20)alkyl or aryl). The solvent used in the reaction may be selected from benzene, toluene, xylene, acetonitrile, THF and the like. The temperature of the reaction may be maintained in the range of 0 to 200° C., preferably in the range of room temperature to boiling temperature of the solvent. The duration of the reaction may be in the range of 2 to 25 hours, preferably 3 to 15 hours.

Another embodiment of the present invention provides a novel intermediate of the formula (Ig), 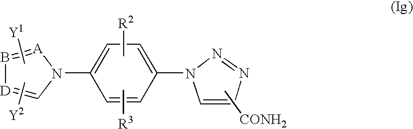
where all symbols are as defined earlier.

Yet another embodiment of the present invention provides a process for the preparation of novel intermediate of formula (Ig), which comprises:

(i) converting the compound of formula (If) 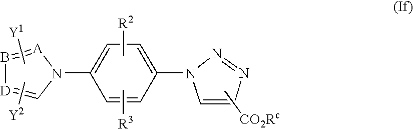
where R^{c}represents substituted or unsubstituted (C1-C20)alkyl group such as methyl, ethyl, n-propyl, iso-propyl and the like; A, B, D, Y¹, Y², R² and R³ are as defined earlier, to a compound of formula (Ig) 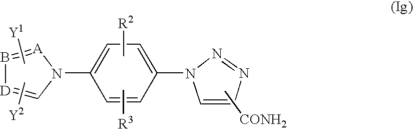
where all symbols are as defined earlier.

The conversion of compound of formula (If) to a compound of formula (Ig) may be carried out in the presence of ammonia solution in water or alcohol. The temperature of the reaction may be in the range of −40 to 50° C., preferably of 0° C. to room temperature. The duration of the reaction may be in the range of 0.5 to 12 hours, preferably 0.5 to 4 hours.

Another embodiment of the present invention provides a novel intermediate of the formula (Ij), 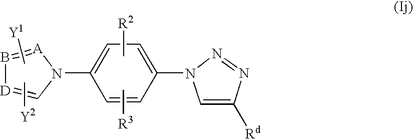
where R^{d} represents substituted or unsubstituted groups selected from —(C1-C20)alkyl, —CO₂R^{c}, —CH₂OH, —CH₂NH₂, —CH₂N(Pthalimide), —CH₂NH—C(═S)S—O(C1-C20)alkyl or —CH₂NH—C(═O)—(C1-C20)alkyl and all other symbols are as defined earlier.

The compound of formula (Ij) represents the compounds of formula (I), when R^{d} represents substituted or unsubstituted groups selected from —(C1-C20)alkyl, —CH₂OH, —CH₂NH₂, —CH₂N(Pthalimide), —CH₂NH—C(═S)S—O(C1-C20)allyl, —CH₂NH—C(═O) (C1-C20)alkyl.

Still yet another embodiment of the present invention provides a process for the preparation of novel intermediate of formula (Ij), which comprises:

(i) converting the compound of formula (Ie), 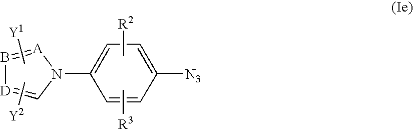
where Z, Y¹, Y², Y³, R² and R³ are as defined earlier, with
≡R^{d} (Ik)
where R^{d} is as defined above, to a compound of formula (Ij) 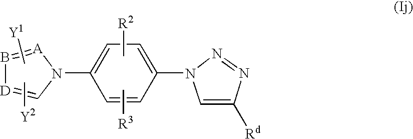
where Z, Y¹, Y², Y³, R^{d}, R² and R³ are as defined earlier.

The compound of formula (Ij) may be prepared by reacting the compound of formula (Ie) with a compound of formula (Ik), in the presence of a base such as triethylamine, ethyldiisopropylamine, DABCO and the like. The reaction may be carried out in the presence of a solvent such as dichloromethane, chloroform, tetrahydrofuran, dimethylformamide, dimethylsulfoxide, acetonitrile and the like. The reaction may be carried out in the presence of Cu (I)I.

It is appreciated that in any of the above-mentioned reactions, any reactive group in the substrate molecule may be protected according to conventional chemical practice. Suitable protecting groups in any of the above mentioned reactions are tertiarybutyldimethylsilyl, methoxymethyl, triphenyl methyl, benzyloxycarbonyl, tetrahydropyran(THP) etc, to protect hydroxyl or phenolic hydroxy group; N-tert-butoxycarbonyl (N-Boc), N-benzyloxycarbonyl (N-Cbz), N-9-fluorenyl methoxy carbonyl (—N-FMOC), benzophenoneimine, propargyloxy carbonyl (POC) etc, for protection of amino or anilino group, acetal protection for aldehyde, ketal protection for ketone and the like. The methods of formation and removal of such protecting groups are those conventional methods appropriate to the molecule being protected.

A method of treating or preventing an bacterial infections in a subject is provided by administering an therapeutically effective amount of compound of formula (I).

The term “therapeutically effective amount” shall mean that amount of a drug or pharmaceutical agent that will elicit the biological or medical response of a tissue, system or patient that is being sought.

The pharmaceutically acceptable salts are prepared by reacting the compounds of formula (I) wherever applicable with 1 to 4 equivalents of a base such as sodium hydroxide, sodium methoxide, sodium hydride, potassium t-butoxide, calcium hydroxide, magnesium hydroxide and the like, in the presence of a solvent like ether, THF, methanol, t-butanol, dioxane, isopropanol, ethanol etc. Mixture of solvents may be used. Organic bases like lysine, arginine, diethanolamine, choline, tromethamine, guanidine and their derivatives etc. may also be used. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid salicylic acid, hydroxynaphthoic acid, ascorbic acid, palmitic acid, succinic acid, benzoic acid, benzenesulfonic acid, tartaric acid and the like in the presence of a solvent like ethyl acetate, ether, alcohols, acetone, THF, dioxane etc. Mixture of solvent may also be used. The salts of amino acid groups and other groups may be prepared by reacting the compounds of formula (I) with the respective groups in the presence of a solvent like alcohols, ketones, ether etc. Mixture of solvents may be used.

The present invention also provides pharmaceutical compositions, containing compounds of the general formula (I), their pharmaceutically acceptable salts The pharmaceutical compositions according to this invention can be used for the treatment of bacterial infections. They can also be used for the treatment of bacterial infections associated with multidrug resistance. The pharmaceutical compositions according to this invention can also be administered prophylatically for the prevention of bacterial infections in a patient at risk of developing a bacterial infection.

The pharmaceutical compositions may be in the forms normally employed, such as tablets, capsules, powders, dispersible granules, cachets, suppositories, syrups, solutions, suspensions and the like, may contain flavorants, sweeteners etc. in suitable solid or liquid carriers or diluents, or in suitable sterile media to form injectable solutions or suspensions. Such compositions typically contain from 0.5 to 90% by weight of active compound, the remainder of the composition being pharmaceutically acceptable carriers, diluents or solvents.

Suitable pharmaceutically acceptable carriers include solid fillers or diluents and sterile aqueous or organic solutions. The active compounds will be present in such pharmaceutical compositions in the amounts sufficient to provide the desired dosage in the range as described above. Thus, for oral administration, the compounds can be combined with a suitable solid, liquid carrier or diluent to form capsules, tablets, powders, syrups, solutions, suspensions and the like. The pharmaceutical compositions, may, if desired, contain additional components such as flavorants, sweeteners, excipients and the like. For parenteral administration, the compounds can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or salts with base of the compounds. The injectable solutions prepared in this manner can then be administered intravenously, intraperitoneally, subcutaneously, or intramuscularly, with intramuscular administration being preferred in humans.

The compounds of the formula (I) or pharmaceutical compositions thereof as defined above are clinically administered to mammals, including human beings, via oral, parenteral and/or topical routes. Administration by the oral route is preferred, being more convenient and avoiding the possible pain and irritation of injection. However, in circumstances where the patient cannot swallow the medication, or absorption following oral administration is impaired, as by disease or other abnormality, it is essential that the drug be administered parenterally. By either route, the dosage is in the range of about 0.1 mg/kg to about 100 mg/kg, more preferably about 3.0 mg/kg to about 50 mg/kg of body weight of the subject per day administered singly or as a divided dose. However, the optimum dosage whether for prevention or treatment for the individual subject being treated will be determined by the person responsible for treatment, Initial dosage may be smaller than the optimum and the daily dosage may be progressively increased during the course of treatment depending on the particular situation. If desired, the daily dose may also be divided into multiple doses for administering, e.g. 2-4 times per day. It is to be understood that the dosages may vary depending upon the requirements of the patient, the severity of the bacterial infection being treated, and the particular compound being used. In a topical treatment an effective amount of compound of formula (I) is admixed in a pharmaceutically acceptable gel or cream vehicle that can be applied to the patient's skin at the area of treatment. Such creams and gels can be prepared by the procedures available in the literature and can include penetration enhancers.

The manner in which the compounds of this invention can be prepared is illustrated in the following examples, which demonstrate the preparation of typical species of the invention. In these examples, the identities of compounds, intermediates and final, were confirmed by infrared, nuclear magnetic spectral analyses as necessary. The examples are for the purpose of illustration only and should not be regarded as limiting the invention in any way.

Preparation 1

### 1-Azido-4-nitrobenzene

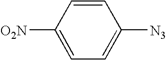

To a solution of p-nitroaniline (5.00 g, 36.2 mmol) in 6N HCl (150 mL), cooled to 0° C., was added sodium nitrite (4.99 g, 72.4 mmol) and stirred at the same temperature for 0.5 hours. A saturated solution of sodium azide (4.70 g, 72.4 mmol) and sodium acetate (59 g, 724 mmol) in water (200 mL) was added dropwise to the above reaction mixture over a period of 0.5 hours. The precipitate formed was filtered and washed repeatedly with water and dried under vacuum. The title azide was obtained as a brown solid (5.60 g, 95%).

¹H NMR (CDCl₃): δ 8.25 (d, J=9.3 Hz, 2H), 7.15 (d, J=9.3 Hz, 2H).

MS (m/e): 165 (M⁺+1), 139, 117.

Preparation 2

### [1-(4-Nitrophenyl)-1H-[1,2,3]triazol-4-yl]-methanol & [1-(4-nitrophenyl)-1H-[1,2,3]triazol-5-yl]-methanol

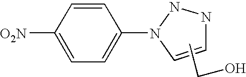

Propargyl alcohol (8.19 g, 146.3 mmol) was added to a solution of 1-azido-4-nitrobenzene (8.00 g, 48.8 mmol), obtained in preparation 1, in toluene (200 mL) and refluxed for 15 hours. Toluene was removed under vacuum on rotavapor to yield the title compounds (mixture of regioisomers) as light brown solid (9.70 g, 90%).

MS (m/e): 221 (M⁺+1).

Preparation 3:

### Methanesulfonic acid [1-(4-nitrophenyl)-1H-[1,2,3]triazol-4-yl]-methyl ester & methanesulfonic acid [1-(4-nitrophenyl)-1H-[1,2,3]triazol-5-yl]-methyl ester

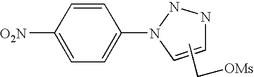

To an ice cooled solution containing a mixture of [1-(4-nitrophenyl)-1H-[1,2,3]triazol-4-yl]-methanol and [1-(4-nitrophenyl)-1H-[1,2,3]triazol-5-yl]-methanol (9.70 g, 44 mmol), obtained in preparation 2, in DMF (50 mL), was added triethylamine (11.47 g, 113.6 mmol) followed by the addition of methanesulfonyl chloride (7.77 g, 68.17 mmol) and the reaction mixture was stirred at the same temperature for 4 hours. The reaction mixture was diluted with cold water (100 mL) and extracted with ethyl acetate (100 mL×3). The combined ethyl acetate extracts were washed with water followed by brine and dried over sodium sulfate. Evaporation of volatiles on rotavapor yielded the title compounds (mixture of regioisomers) as yellow oil (10 g, 76%).

Preparation 4:

### 4-Azidomethyl-1-(4-nitrophenyl)-1H-[1,2,3] triazole

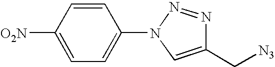

Sodium azide (4.92 g, 75.8 mmol) was added to a solution containing a mixture of methanesulfonic acid [1-(4-nitrophenyl)-1H-[1,2,3]triazol-4-yl]-methyl ester & methanesulfonic acid [1-(4-nitrophenyl)-1H-[1,2,3]triazol-5-yl]-methyl ester (10 g, 37.8 mmol), obtained in preparation 3, in DMF (50 mL) and the reaction mixture was heated to 90° C. for 1 h. The reaction mixture was then cooled to room temperature and diluted with ethyl acetate (250 mL). The organic layer was washed with water followed by brine and dried over sodium sulfate. Removal of volatiles on rotavapor and purification of the resulting residue by column chromatography (silica gel) yielded the title compound as yellow solid (3.60 g, 44%).

¹H NMR (CDCl₃): δ 8.45 (d, J=8.8 Hz, 2H), 8.16 (s, 1H), 7.99 (d, J=8.8 Hz, 2H), 4.60 (s, 2H); MS (m/e): 246 (M⁺+1)

Preparation 5:

### C-[1-(4-Nitrophenyl)-1H-[1,2,3]triazol-4-yl]-methylamine

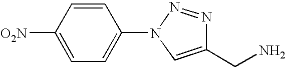

A mixture of 4-azidomethyl-1-(4-nitrophenyl)-1H-[1,2,3]triazole (3.60 g, 14.7 mmol), obtained in preparation 4, and triphenylphosphine (4.23 g, 16 mmol) in THF (50 mL) was stirred at room temperature for 4 hours. It was then warmed to 40° C. after the addition of 5 mL of water and allowed to stir at the same temperature for 16 hours. The reaction mixture was then diluted with water and extracted with ethyl acetate (100 mL×2). Combined ethyl acetate extracts were washed with water followed by brine and dried over sodium sulfate. Evaporation of volatiles and purification of the resulting residue by column chromatography (silica gel, CHCl₃/MeOH, 9:1) yielded the title amine (1 g, 32%).

¹H NMR (CDCl₃): δ 8.42 (d, J=9.3 Hz, 2H), 8.00 (d, J=9.3 Hz, 2H), 7.83 (s, 1H), 4.01 (s, 2H); MS (m/e): 220 (M⁺+1), 190;

Preparation 6:

### [1-(4-Nitrophenyl)-1H-[1,2,3]triazol-4-ylmethyl]-thiocarbamic acid O-methyl ester

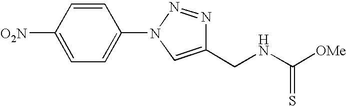

To an ice-cooled solution of C-[1-(4-nitrophenyl)-1H-[1,2,3]triazol-4-yl]-methylamine (1 g, 4.5 mmol), obtained in preparation 5, in chloroform (40 mL) was added a saturated solution of sodium bicarbonate followed by the addition of thiophosgene (0.63 grams, 5.5 mmol) and stirred at room temperature for 0.5 hours. The reaction mixture was then diluted with ethyl acetate (100 mL) and the organic layer was washed with water followed by brine and dried over sodium sulfate. Evaporation of volatiles left a residue, which was refluxed with methanol (30 mL) for 16 hours. Removal of methanol on rotavapor and purification of the resulting residue through a silica gel column (ethyl acetate/chloroform, 1:9) yielded the title compound as white solid (800 mg, 70%).

¹H NMR (CDCl₃+DMSO-d₆): δ 9.05 (bs, 1H), 8.41 (d, J=8.9 Hz, 2H), 8.00 (d, J=8.9 Hz, 2H), 4.91 & 4.65 (2 d, J=5.9 Hz, 2H, rotamers in a ratio of 4:1), 4.12 & 4.09 (2 s, 3H, rotamers in a ratio of 1:4); MS (m/e): 294 (M⁺+1), 262, 175.

Preparation 7:

### [1-(4-Aminophenyl)-1H-[1,2,3]triazol-4-ylmethyl]-thiocarbamic acid O-methyl ester

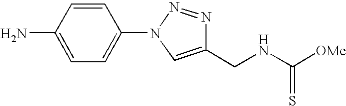

A solution of [1-(4-nitrophenyl)-1H-[1,2,3]triazol-4-ylmethyl]-thiocarbamic acid O-methyl ester (800 mg, 2.73 mmol), obtained in preparation 6, in ethanol (10 mL) was added to a warm solution of ammonium chloride (1.46 grams, 27.30 mmol) in ethanol (30 mL) and water (15 mL). Iron powder (0.45 grams, 8.19 mmol) was added slowly in portion to the reaction mixture over a period of 0.5 hours and stirred for another 0.5 hours at 95° C. The reaction mixture was then filtered to remove the black material and diluted with ethyl acetate (100 mL). The organic phase was washed with water followed by brine and dried over sodium sulfate. Evaporation of volatiles on rotavapor yielded the title compound as white solid (600 mg, 84%).

¹H NMR (CDCl₃+DMSO-d₆): δ 9.22 (bs, 1H), D₂O exchangeable)), 8.12 & 8.09 (2s, 1H, rotamers in a ratio of 4:1), 7.41 (d, J=8.6 Hz, 2H), 6.74 (d, J=8.6 Hz, 2H), 4.82 & 4.79 (2d, J=5.9 Hz, 2H, rotamers in a ratio of 4:1), 4.03 & 3.95 (2s, 3H, rotamers in a ratio of 1:4); MS (m/e): 264 (M⁺+1), 232, 145.

Preparation 8:

### 2-(2-Fluoro-4-nitro-phenyl)-isoindole-1,3-dione

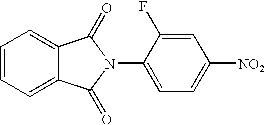

To a solution of 3,4-difluoronitrobenzene (10 grams, 62.8 mmol) in DMF (25 mL) was added potassium pthalimide (9.18 grams, 62.8 mmol) and heated to 100° C for 4 hours. The reaction mixture was then poured onto crushed ice and the resulting solid was filtered. The precipitate was washed with water and dried under vacuum to get the title compound as crystalline yellow solid.

¹H NMR (DMSO-d₆): δ 8.41 (d, J=9.7 Hz, 1H), 8.31 (d, J=8.9 Hz, 1H), 8.08-7.90 (m, 5H); MS (m/e): 287 (M⁺+1); IR (KBr, cm⁻¹): 1737, 1718, 1521, 1510, 1377.

Preparation 9:

### 2-(4-Amino-2-fluoro-phenyl)-isoindole-1,3-dione

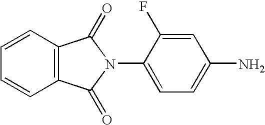

To a solution of 2-(2-fluoro-4-nitro-phenyl)-isoindole-1,3-dione (3.80 grams, 13.6 mmol), obtained in preparation 8, in methanol (15 mL) and THF (15 mL) was added ammonium formate (6.80 grams, 108.6 mmol) at 0° C. Palladium on charcoal (10%, 500 mg) was added and stirred for 1 hours at room temperature. Filtration over a pad of celite followed by evaporation of filtrate left a pasty mass, which was diluted with water and extracted with ethyl acetate (150 mL×2). The combined ethyl acetate extracts were washed with water followed by brine and dried over sodium sulfate. Evaporation of volatiles on a rotavapor yielded the title compound as dark brown solid (2.85 grams, 86%).

¹H NMR (CDCl₃): δ 7.96-7.76 (m, 4H), 7.08 (t, J=8.2 Hz, 2H), 6.53 (d, J=10.2 Hz, 2H); MS (m/e): 257 (M⁺+1), 237, 148, 127; IR (KBr, cm⁻¹): 1706, 1634, 1522, 1397.

Preparation 10:

### 2-(4-Azido-2-fluoro-phenyl)-isoindole-1,3-dione

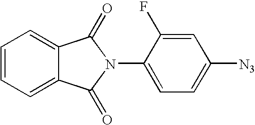

To an ice cooled solution of 2-(4-amino-2-fluorophenyl)-isoindole-1,3-dione (500 mg, 2 mmol), obtained in preparation 9, in 6N HCl (15 mL) was added sodium nitrite and stirred for 1 hours. A solution of sodium azide (65 mg, 1 mmol) and sodium acetate (3.28 grams, 20 mmol) in water (50 mL) was added and stirred for few min. The reaction mixture was then extracted with ethyl acetate (50 mL×2). The combined organic layer was washed with water followed by brine and dried over sodium sulfate. Evaporation of volatiles yielded the title compound as brown solid (518 mg, 92%).

¹H NMR (CDCl₃): δ 7.99-7.95 (m, 2H), 7.90-7.79 (m, 2H), 7.35 (t, J=8.3 Hz, 1H), 6.99-6.93 (m, 2H); MS (m/e): 283 (M⁺+1), 254, 148; IR (KBr, cm⁻¹): 2113, 1716, and 1515.

Preparation 11:

### 4-Azido-2-fluoro-phenylamine

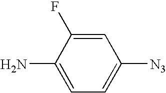

To a solution of 2-(4-azido-2-fluoro-phenyl)-isoindole-1,3-dione (500 mg, 1.77 mmol), obtained in preparation 10, in methanol (10 mL) was added hydrazine hydrate (532 mg, 10.3 mmol) and refluxed for 2 hours. Evaporation of methanol on rotavapor and purification of the resulting residue through silica gel column (ethyl acetate/pet. ether, 1:5) yielded the title compound (198 mg, 74%).

¹H NMR (CDCl₃): δ 6.81-6.63 (m, 3H), 3.67 (bs, 2H); MS (m/e): 153 (M⁺+1); IR (neat, cm⁻¹): 2114, 1681, 1500.

Preparation 12:

### 1-(4-Azido-2-fluorophenyl)-1H-pyrrole

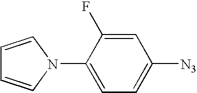

To a solution of 4-azido-2-fluoro-phenylamine (100 mg, 0.66 mmol), obtained in preparation 11, in glacial acetic acid (2 mL) was added 2,4-dimethoxytetrahydrofuran (87 mg, 0.66 mmol) and heated to 114° C. for 4 hours. The reaction mixture was then treated with saturated sodium bicarbonate solution and extracted with ethyl acetate (50 mL×2). Combined ethyl acetate extracts were washed with water followed by brine and dried over sodium sulfate. Evaporation of volatiles yielded the title compound as dark brown semi solid (98 mg, 74%).

¹H NMR (CDCl₃): δ 7.37 (t, J=8.5 Hz, 1H), 6.87-6.58 (m, 4H), 6.35-6.34 (m, 2H); MS (m/e): 203 (M⁺+1), 177; IR (KBr, cm⁻¹): 2113, 1591, 1519, 1305.

Preparation 13:

### 1-(4-Azido-2-fluoro-phenyl)-1H-pyrrole-3-carboxaldehyde

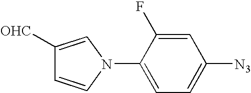

To a solution of 4-azido-2-fluoro-phenylamine (100 mg, 0.66 mmol), obtained in preparation 11, in glacial acetic acid (2 mL) was added 2,5-dimethoxy tetrahydrofuran-3-carboxaldehyde (95.6 mg, 0.72 mmol) and heated to 110° C. for 3 hours. The reaction mixture was diluted with water (25 mL) and neutralized with saturated solution of sodium bicarbonate. The aqueous layer was then extracted with ethyl acetate (50 mL×3) and the combined extracts were washed with water followed by brine. After drying over sodium sulfate, ethyl acetate was removed on rotavapor and the resulting residue was purified by passing through a silica gel column (ethyl acetate/pet ether, 1:2) to yield the title compound as light brown solid (135 mg, 89%).

¹H NMR (CDCl₃): δ 9.85 (s, 1H) 7.58 (s, 1H), 7.40 (t, J=8.3, 2H), 6.97-6.74 (m, 3H); MS (m/e): 231 (M⁺+1), 205; IR (neat, cm⁻¹): 2115, 1674, 1522.

Preparation 14:

### 1-(4-Azido-2-fluoro-phenyl)-1H-pyrrole-3-carboxaldehyde oxime

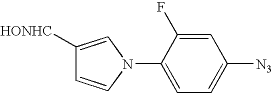

To a solution of 1-(4-azido-2-fluoro-phenyl)-1H-pyrrole-3-carboxaldehyde (135 mg, 0.59 mmol), obtained in preparation 13, in a mixture of dichloromethane/methanol (1:1, 3 mL) was added hydroxylamine hydrochloride (61.2 mg, 0.88 mmol) followed by the addition of potassium carbonate (80.5 mg, 0.58 mmol). The reaction mixture was stirred at room temperature for 4 hours. It was then diluted with 50 mL of water and extracted with ethyl acetate (75 mL×3). Combined ethyl acetate extracts were washed with brine and dried over sodium sulfate. Evaporation of volatiles in a rotavapor yielded the title compound (130 mg, 90%).

¹H NMR (CDCl₃): δ 8.11 (s, 1H), 7.78 (s, 1H), 7.42-7.28 (m, 2H), 6.98-6.81 (m, 3H), 6.64 (bs, 1H); MS (m/e): 247 (M⁺), 246, 228, 217, 202; IR (KBr, cm⁻¹): 3216, 2116, 1590, 1520, 1307, 1218, 758.

Preparation 15:

### 1-(4-Azido-2-fluoro-phenyl)-1H-pyrrole-3-carbonitrile

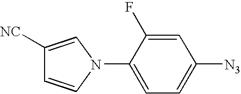

To a solution of 1-(4-azido-2-fluoro-phenyl)-1H-pyrrole-3-carboxaldehyde oxime (200 mg, 0.82 mmol), obtained in preparation 14, in THF (4 mL) was added Burgess reagent [(Methoxycarbonylsulfamoyl)triethylammonium hydroxide, inner salt, 293 mg, 1.23 mmol] and refluxed at 65° C. for 4 hours. It was then concentrated on a rotavapor to get a crude product, which was purified by silica gel column chromatography with a mobile phase petether:ethylacetate (1:1) to get the title compound (81 mg, 44%).

¹H NMR (CDCl₃): 7.42-7.31 (m, 2H), 6.54 (d, J=9.4 Hz, 3H), 6.59 (s, 1H); MS (m/e):228 (M⁺+1), 202; IR (neat, cm⁻¹): 3440, 2923, 2133, 1522.

Preparation 16:

### 2-(2,6-Difluoro-4-nitrophenyl)-isoindole-1,3-dione

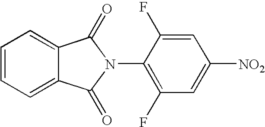

To a solution of 3,4,5-trifluoronitrobenzene (10 grams, 56.5 mmol) in DMF (50 mL) was added potassium pthalimide (8.247 grams, 56.5 mmol) and heated to 100° C. for 4 h. The reaction mixture was then poured onto crushed ice and the resulting solid was filtered. The precipitate was washed with water and dried under vacuum to get the compound as crystalline yellow solid (10 grams, 61%).

¹H NMR (CDCl₃): δ 8.11-7.92 (m, 4H), 7.90-7.81 (m, 2H); MS (m/e): 305(M⁺+1), 275, 148; IR (KBr, cm⁻¹): 3095, 1742, 1718, 1532, 1510, 1371, 1346.

Preparation 17:

### 2-(4-Amino-2,6-difluorophenyl)-isoindole-1,3-dione

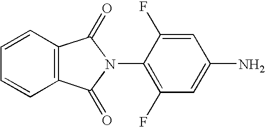

To a solution of 2-(2,6-difluoro-4-nitrophenyl)-isoindole-1,3-dione (10 g, 32.89 mmol), obtained in preparation 16, in methanol (45 mL) and THF (15 mL) was added palladium on charcoal (10%, 1.82 grams) and stirred for 4 hours at room temperature. The reaction mixture was filtered to get rid off the catalyst. Evaporation of volatiles in a rotavapor yielded the title compound as dark brown solid (8.77 grams, 97%).

¹H NMR (CDCl₃+DMSO-d₆): δ 8.01-7.81 (m, 4H), 6.67 (d, J=9.3 Hz, 2H), 4.92 (bs, 2H); MS (m/e): 275 (M⁺+1), 230, 148; IR (KBr, cm⁻¹): 3423, 3315, 2925, 1714, 1524.

Preparation 18:

### 2-(4-Azido-2,6-difluorophenyl)-isoindole-1,3-dione

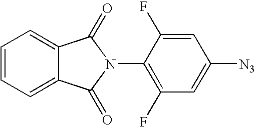

To an ice cooled solution of 2-(4-amino-2,6-difluorophenyl)-isoindole-1,3-dione (500 mg, 1.82 mmol), obtained in preparation 17, in 6N HCl (10 mL) was added sodium nitrite and stirred for 4 hours. A solution of sodium azide (136.8 mg, 1.82 mmol) and sodium acetate (2.99 grams, 36.49 mmol) in water (100 mL) was added and stirred for few min. The reaction mixture was then extracted with ethyl acetate (50 mL×2). The combined organic layer was washed with water followed by brine and dried over sodium sulfate. Evaporation of volatiles yielded the title compound as brown solid (82 mg, 15%).

¹H NMR (CDCl₃): δ 8.05-7.96 (m, 2H), 7.95-7.73 (m, 2H), 6.78 (d, J=8.1 Hz, 2H); MS (m/e): 301 (M⁺+1), 275, 272, 253, 104; IR (KBr, cm⁻¹): 2119, 1740, 1716, 1518.

Preparation 19:

### 4-Azido-2,6-difluoro-phenylamine

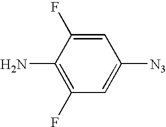

To a solution of 2-(4-azido-2,6-difluoro-phenyl)-isoindole-1,3-dione (1.5 grams, 1.53 mmol), obtained in preparation 18, in methanol (10 mL) was added ethylene diamine (1.5 grams, 25 mmol) and stirred at room temperature. Evaporation of methanol on rotavapor and purification of the resulting residue through silica gel column (ethyl acetate/pet. ether: 1:5) yielded the title compound (707 mg, 83%).

¹H NMR (CDCl₃): δ 6.58 (d, J=6.8 Hz, 2H), 3.66 (bs, 2H); MS (m/e): 171 (M⁺+1), 143, 128; IR (neat, cm⁻¹): 3419, 3322, 2118, 1600, 1519.

Preparation 20:

### 1-(4-Azido-2,6-difluoro-phenyl)-1H-pyrrole-3-carboxaldehyde

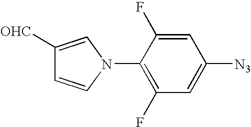

To a solution of 4-azido-2,6-difluoro-phenylamine (180 mg, 0.6 mmol), obtained in preparation 19, in glacial acetic acid (2 mL) was added 2,5-dimethoxy tetrahydrofuran-3-carboxaldehyde (199 mg, 1.48 mmol) and heated to 100° C. for 4 hours. The reaction mixture was diluted with water (25 mL) and neutralized with saturated solution of sodium bicarbonate. The aqueous layer was then extracted with ethyl acetate (50 mL×3) and the combined extracts were washed with water followed by brine. After drying over sodium sulfate, ethyl acetate was removed on rotavapor and the resulting residue was purified by passing through a silica gel column (ethyl acetate/pet ether, 1:2) to yield the title compound as light brown solid (296 mg, 80%).

¹HNMR (CDCl₃): δ 9.87 (s, 1H), 7.47 (s, 1H), 6.95-6.75 (m, 4H); MS (m/e): 249 (M⁺+1), 220; IR (KBr, cm⁻¹): 2924, 2118, 1679, 1529.

Preparation 21:

### 1-(4-Azido-2,6-difluoro-phenyl)-1H-pyrrole-3-carboxaldehyde oxime

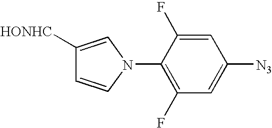

To a solution of 1-(4-azido-2,6-difluoro-phenyl)-1H-pyrrole-3-carboxaldehyde (456 mg, 1.83 mmol), obtained in preparation 20, in a mixture of dichloromethane/methanol (1:1, 3 mL) was added hydroxylamine hydrochloride (190 mg, 2.74 mmol) followed by the addition of potassium carbonate (252 mg, 1.83 mmol). The reaction mixture was stirred at room temperature for 4 hours. It was then diluted with 50 mL of water and extracted with ethyl acetate (75 mL×3). Combined ethyl acetate extracts were washed with brine and dried over sodium sulfate. Evaporation of volatiles in a rotavapor yielded the title compound (323 mg, 67%).

¹HNMR (CDCl₃): δ 8.11 (s, 1H), 7.68 (s, 1H), 7.06 (s, 1H), 6.91-6.58 (m, 4H); MS (m/e): 264 (M⁺+1), 246, 236, 218; IR (neat, cm⁻¹): 3216, 2926, 2117, 1527, 1238, 1044.

Preparation 22:

### 1-(4-Azido-2,6-difluoro-phenyl)-1H-pyrrole-3-carbonitrile

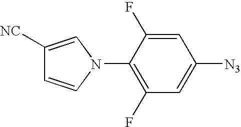

To a solution of 1-(4-azido-2,6-difluoro-phenyl)-1H-pyrrole-3-carboxaldehyde oxime (323 mg, 1.22 mmol), obtained in preparation 21, in THF (3 mL) was added Burgess reagent [(Methoxycarbonylsulfamoyl)triethylammonium hydroxide, inner salt, 314 mg, 1.32 mmol] and refluxed at 65° C. for 4 hours. It was then concentrated on a rotavapor to get a crude product, which was purified by silica gel column chromatography with a mobile phase petether:ethylacetate (1:4) to get the title compound (166 mg, 55%).

¹HNMR (CDCl₃): δ 7.38-7.22 (m, 2H), 6.90-6.71 (m, 2H), 6.63 (s, 1H); MS (m/e): 246 (M⁺+1), 220, 162, 151.

Preparation 23:

### 1-(2,6-Difluoro-4-nitrophenyl)-1H-imidazole

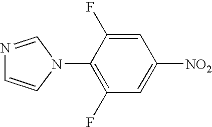

To a suspension of anhydrous potassium carbonate (17.30 grams, 112.80 mmol) in dry DMF (100 mL) was added 3,4,5-trifluoronitrobenzene (10 grams, 56.40 mmol) followed by the addition of imidazole (4.60 grams, 67.6 mmol) and heated to 80° C. for 1 hours. The reaction mixture was then poured onto crushed ice and the resulting solid was filtered. The solid collected was washed with water and dried to yield the title compound as yellow powder (8 grams, 62%).

¹H NMR (CDCl₃): δ 8.03 (d, J=7.5 Hz, 2H), 7.80 (s, 1H), 7.31 (s, 2H); MS (m/e): 226 (M⁺+1), 196; IR (1KBr, cm⁻¹): 3128, 3027, 1520, 1045.

Preparation 24:

### 3,5-Difluoro-4-imidazol-1-yl-phenylamine

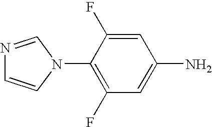

To a mixture of nickel chloride hexahydrate (16.49 grams, 71.10 mmol) and 1-(2,6-difluoro-4-nitrophenyl)-1H-imidazole (8 grams, 35.50 mmol), obtained in preparation 23, in methanol (100 mL) was added sodium borohydride (4 grams, 106.60 mmol) in portion and stirred for 0.5 hours. The reaction mixture was diluted with ethyl acetate (500 mL). The organic layer was washed with water followed by brine and dried over sodium sulfate. Evaporation of volatiles yielded the title compound as brown solid (5.90 grams, 85%).

¹H NMR (CDCl₃): δ 7.76 (s, 1H), 7.29 (s, 1H), 7.01 (s, 1H), 6.37 (d, J=10.7 Hz, 2H), 6.02 (bs, 2H); MS (m/e): 196 (M⁺+1); IR (KBr, cm⁻¹): 3423, 3339, 3221, 1647.

Preparation 25:

### 1-(4-Azido-2,6-difluorophenyl)-1H-imidazole

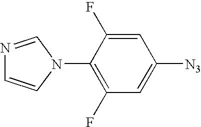

To an ice cooled solution of 3,5-difluoro-4-imidazol-1-yl-phenylamine (1 grams, 5.12 mmol), obtained in preparation 24, in 6N HCl (20 mL), was added sodium nitrite (0.70 grams, 10.25 mmol) and stirred for 1 hours. Then a saturated solution of sodium azide (0.66 grams, 10.25 mmol) and sodium acetate (8.60 grams, 102.50 mmol) in water (50 mL) was added slowly at the same temperature. The reaction mixture was diluted with ethyl acetate (250 mL). The organic layer was washed with water followed by brine and dried over sodium sulfate. Evaporation of volatiles yielded the title compound as light brown solid (1 grams, 90%).

¹H NMR (CDCl₃): δ 7.90 (s, 1H), 7.46 (s, 1H), 7.32 (d, J=8.6 Hz, 2H), 7.11 (s, 1H); MS (m/e): 222 (M⁺+1), 196; IR (KBr, cm⁻¹): 2126.

Preparation 26:

### 1-(2-Fluoro-4-nitrophenyl)-1H-imidazole

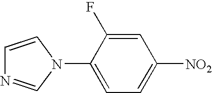

To a suspension of anhydrous potassium carbonate (2.60 grams, 15.6 mmol) in dry DMF (15 mL) was added 3,4-difluoronitrobenzene (2.0 grams, 12.6 mmol) followed by the addition of imidazole (1.03 grams, 15.1 mmol) and heated to 80° C. for 1 hours. The reaction mixture was then poured onto crushed ice and the resulting solid was filtered. The solid collected was washed with water and dried to yield the title compound as yellow powder (2 grams, 74%).

¹H NMR (CDCl₃): δ 8.15 (d, J=8.8 Hz, 2H), 7.90 (s, 1H), 7.59 (t, J=8.2 Hz, 1H), 7.27 (d, J=4.8 Hz, 2H); MS (m/e): 208 (M⁺+1), 194, 178.

Preparation 27:

### 3-Fluoro-4-imidazol-1-yl-phenylamine

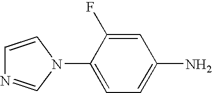

To a mixture of nickel chloride hexahydrate (4.83 grams, 19.3 mmol) and 1-(2-fluoro-4-nitrophenyl)-1H-imidazole (2 grams, 9.7 mmol), obtained in preparation 26, in methanol (10 mL) was added sodium borohydride (1.1 grams, 29.0 mmol) in portion and stirred for 0.5 hours. The reaction mixture was diluted with ethyl acetate (100 mL). The organic layer was washed with water followed by brine and dried over sodium sulfate. Evaporation of volatiles yielded the title compound as brown oil (1.5 grams, 88%).

¹H NMR (CDCl₃): δ 7.79 (s, 1H), 7.38 (t, J=8.1 Hz, 1H), 7.23 (s, 2H), 6.97-6.92 (m, 2H); MS (m/e): 178 (M⁺+1), 176.

Preparation 28:

### 1-(4-Azido-2-fluorophenyl)-1H-imidazole

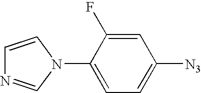

To an ice cooled solution of 3-fluoro-4-imidazol-1-yl-phenylamine (2.0 grams, 11.3 mmol), obtained in preparation 27, in 6N HCl (10 mL), was added sodium nitrite (1.5 grams, 22 mmol) and stirred for 1 hours. Then a saturated solution of sodium azide (1.4 grams, 22 mmol) and sodium acetate (18.5 grams, 226 mmol) in water (50 mL) was added slowly at the same temperature. The reaction mixture was diluted with ethyl acetate (100 mL). The organic layer was washed with water followed by brine and dried over sodium sulfate. Evaporation of volatiles yielded the title compound as light brown solid (2 grams, 83%).

¹H NMR (CDCl₃): δ 7.80 (s, 1H), 7.38 (t, J=8.1 Hz, 1H), 7.20 (s, 2H), 6.97-6.92 (m, 2H); MS (m/e): 204 (M⁺+1), 178, 176; IR (KBr, cm⁻¹): 2114.

Preparation 29:

### 1-(2-Fluoro-4-nitrophenyl)-1H-pyrazole

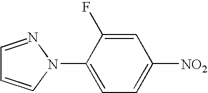

To a suspension of anhydrous potassium carbonate (4.30 grams, 31.10 mmol) in DMF (20 mL) was added 3,4-difluoronitrobenzene (2.50 grams, 15.70 mmol) followed by the addition of pyrazole (1.28 grams, 18.80 mmol) and heated to 80° C. for 1 hours. The reaction mixture was poured onto crushed ice and the precipitate was filtered off. It was then washed with water and dried under vacuum to yield the title compound as yellow solid (3.00 grams, 86%).

¹H NMR (DMSO-d₆): δ 8.53-8.25 (m, 4H), 7.94 (s, 1H), 6.69 (s, 1H); MS (m/e): 208 (M⁺+1); IR (KBr, cm⁻¹): 3441, 3146, 2925, 2119, 1341.

Preparation 30:

### 3-Fluoro-4-pyrazol-1-yl-phenylamine

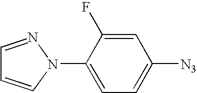

To a mixture of nickel chloride hexahydrate (6.70 grams, 28.80 mmol) and 1-(2-fluoro-4-nitrophenyl)-1H-pyrazole (3.00 grams, 14.49 mmol), obtained in preparation 29, in methanol (50 mL) was added sodium borohydride (1.65 grams, 43.40 mmol) in portion and stirred for 0.5 hours. The reaction mixture was diluted with ethyl acetate (500 mL) and the organic layer was washed with water followed by brine and dried over sodium sulfate. Evaporation of volatiles yielded the title compound as brown solid (2.30 grams, 90%).

¹H NMR (CDCl₃): δ 7.81 (s, 1H), 7.79 (s, 1H), 7.55 (t, J=8.6 Hz, 1H), 6.67-6.42 (m, 3H), 3.80 (bs, 2H); MS (m/e): 178 (M⁺+1); IR (neat, cm⁻¹): 3351, 1633, 1532.

Preparation 31:

### 1-(4-Azido-2-fluoro-phenyl)-1H-pyrazole

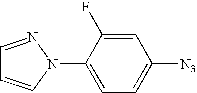

Sodium nitrite (1.79 grams, 25.80 mmol) was slowly added to an ice cooled solution of 3-fluoro-4-pyrazol-1-yl-phenylamine (2.30 grams, 12.90 mmol), obtained in preparation 30, in 6N HCl (20 mL), and stirred for 1 hours. Then a saturated solution of sodium azide (1.68 grams, 25.80 mmol) and sodium acetate (21.80 grams, 258 mmol) in water (90 mL) was added slowly at the same temperature. The reaction mixture was diluted with ethyl acetate (500 mL). The organic layer was washed with water followed by brine and dried over sodium sulfate. Evaporation of volatiles yielded the title compound as brown viscous oil (2.20 grams, 84%).

¹H NMR (CDCl₃): δ 8.05-7.81 (m, 2H), 7.76 (s, 1H), 7.09-6.82 (m, 2H), 6.55 (s, 1H); MS (m/e): 204 (M⁺+1), 167; IR (KBr, cm⁻¹): 2115.

Preparation 32:

### [1-(3-Fluoro-4-pyrazol-1-ylphenyl)-1H-[1,2,3]triazol-4-yl]-methanol & [1-(3-fluoro-4-pyrazol-1-ylphenyl)-1H-1,2,3]triazol-5-yl]-methanol

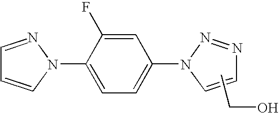

Propargyl alcohol (1.24 grams, 22.10 mmol) was added to a solution of 1-(4-azido-2-fluoro-phenyl)-1H-pyrazole (1.50 grams, 7.38 mmol), obtained in preparation 31, in toluene (50 mL) and refluxed for 16 hours. Removal of toluene under vacuum on rotavapor yielded a mixture of regioisomers as viscous liquid (1.60 grams, 85%).

Preparation 33:

### Methanesulfonic acid [1-(3-fluoro-4-pyrazol-1-ylphenyl)-1H-[1,2,3]triazol-4-yl]-methyl ester & methanesulfonic acid [1-[3-fluoro-4-pyrazol-1-ylphenyl]-1H-[1,2,3]triazol-5-yl]-methyl ester

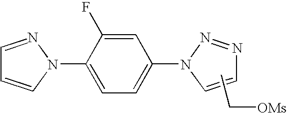

Triethyl amine (1.24 grams, 12.20 mmol) was added to a solution of [1-(3-fluoro-4-pyrazol-1-ylphenyl)-1H-[1,2,3]triazol-4-yl]-methanol & [1-(3-fluoro-4-pyrazol-1-ylphenyl)-1H-[1,2,3]triazol-5-yl]-methanol (1.60 grams, 6.17 mmol), obtained in preparation 32, in dichoromethane (30 mL) followed by the addition of methanesulfonyl chloride (1.05 grams, 9.20 mmol) at 0° C. and stirred for 1 hours. The reaction mixture was diluted with dichloromethane (250 mL). The organic layer was washed with water followed by brine and dried over sodium sulfate. Evaporation of volatiles yielded the title compounds as mixture of regioisomers (1.70 grams, 81%).

Preparation 34:

### 1-(2-Fluoro-4-nitrophenyl)-1H-[1,2,4]-triazole

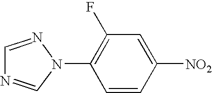

To a suspension of anhydrous potassium bicarbonate (8.30 grams, 60 mmol) in DMF (50 mL) was added 3,4-difluoronitrobenzene (5.0 grams, 31.4 mmol) followed by the addition of [1,2,4]-triazole (2.6 grams, 37.6 mmol) and heated to 80° C. for 1 hours. The reaction mixture was poured onto crushed ice and the precipitate was filtered off. It was then washed with water and dried under vacuum to yield the title compound as white solid (5.85 grams, 90%).

¹H NMR (CDCl₃): δ 8.85 (s, J=2.4 Hz, 1H), 8.32-8.11 (m, 4H); MS (m/e): 209 (M++1), 195; IR (KBr, cm⁻⁴): 1533, 1510; 1346.

Preparation 35:

### 3-Fluoro-4-(1H-[1,2,4]-triazol-1-yl-phenylamine

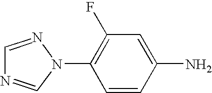

To a mixture of nickel chloride hexahydrate (6.7 grams, 28.8 mmol) and 1-(2-fluoro-4-nitrophenyl)-1H-[1,2,4]-triazole (3.0 grams, 14.4 mmol), obtained in preparation 34, in methanol (70 mL) was added sodium borohydride (1.65 grams, 43.3 mmol) in portion and stirred for 0.5 hours. The reaction mixture was then diluted with ethyl acetate (150 mL) and the organic layer was washed with water followed by brine and dried over sodium sulfate. Evaporation of volatiles yielded the title compound as light brown solid (2.2 grams, 85%).

¹H NMR (CDCl₃): δ 8.48 (d, J=1.9 Hz, 1H), 8.11 (s, 1H), 7.54 (t, J=8.5 Hz, 1H), 6.59 (s, 1H), 6.54 (d, J=2.9 Hz, 1H), 4.01 (bs, 2H); MS (m/e): 179 (M⁺+1), 124; IR (KBr, cm⁻¹): 3376, 3225, 1630, 1527.

Preparation 36:

### 1-(4-Azido-2-fluorophenyl)-1H-[1,2,4]triazole

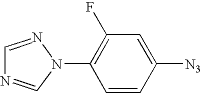

Sodium nitrite (1.55 grams, 22.5 mmol) was slowly added to an ice cooled solution of 3-fluoro-4-(1H-[1,2,4]-triazol-1-yl)phenyl amine (2.0 grams, 11.0 mmol), obtained in preparation 35, in 6N HCl (20 mL), and stirred for 1 hours. A saturated solution of sodium azide (1.43 grams, 22.0 mmol) and sodium acetate (18.50 grams, 220 mmol) in water (50 mL) was added slowly at the same temperature. The reaction mixture was diluted with ethyl acetate (500 mL). The organic layer was washed with water followed by brine and dried over sodium sulfate. Evaporation of volatiles yielded the title compound as light brown solid (2.0 grams, 87%).

¹H NMR (CDCl₃): δ 8.62 (d, J=2.9 Hz, 1H), 8.12 (s, 1H), 7.90 (t, J=8.5 Hz, 1H), 7.05-6.85 (m, 2H); MS (m/e): 206 (M⁺+2), 205, 177, 176; IR (KBr, cm⁻¹): 2924, 2119, 1515, 1289.

Preparation 37:

### [1-(4-Amino-3,5-difluoro-phenyl)-1H-[1,2,3]triazol-4-ylmethyl]-thiocarbamic acid O-methyl ester

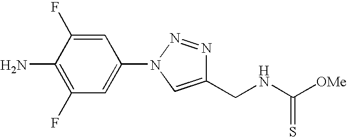

To a solution of {1-[4-(1,3-dioxo-1,3-dihydro-isoindol-2-yl)-3,5-difluoro-phenyl]-1H-[1,2,3]triazol-4-ylmethyl}-thiocarbamic acid O-methyl ester (663 mg, 1.54 mmol), in methanol (3 mL) was added ethylene diamine (555 mg, 9.2 mmol) and stirred at room temperature for 3 hours. Removal of methanol on rotavapor and purification of the resulting residue through a silica gel column (ethyl acetate/pet. ether, 3:1) yielded the title compound as brown colour solid (402 mg, 88%)

¹H NMR(CDCl₃): δ 8.70 (bs, 1H), 8.11 & 7.97 (2 s, 1H, rotamers in a ratio of 4:1), 7.28 (d, J=7.3 Hz, 2H), 4.86 & 4.58 (2 d, J=5.8, 2H, rotamers in a ratio of 4:1), 4.4 (s, 2H), 4.07 & 3.98 (2 s, 3H, rotamers in a ratio of 1:4); MS (m/e): 300(M⁺+1), 270, 181, 128; IR (KBr, cm⁻¹): 3400, 3307, 3198, 2925, 1532, 1477.

Preparation 38:

### 1-(4-Azido-2,6-difluorophenyl)-1H-pyrrole

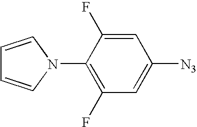

The title compound was prepared from 4-azido-2,6-difluoro-phenylamine, obtained in preparation 19, and 2,5-dimethoxy tetrahydrofuran, following the procedure reported in preparation 12.

¹H NMR (CDCl₃): δ 6.85 (s, 2H), 6.73 (d, J=8.6 Hz, 2H), 6.36 (s, 2H); MS (m/e): 221 (M⁺+1), 195; IR (KBr, cm⁻¹): 2117, 1640, 1589, 1527.

Preparation 39:

### [1-(2-fluoro-4-nitro-phenyl)-1H-imidazol-4-yl]-methanol

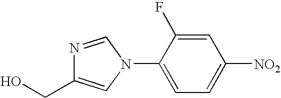

3,4-Difluoronitrobenzene (2 grams, 12.5 mmol) and 4-(hydroxymethyl)-imidazole hydrochloride (1.86 grams, 13.8 mmol) were taken together in N,N-diisopropylethylamine (25 mL) and heated to 120° C. for 12 hours. Two layers were formed after the reaction mixture was allowed to cool to room temperature. Upper layer was decanted and ethyl acetate (500 mL) was added to the remaining portion. The ethyl acetate layer was washed with water followed by brine and dried over sodium sulfate. Evaporation of solvent yielded the tilte compound as brown solid (2 grams, 67%).

¹H NMR (DMSO-d⁶): δ 8.43 (dd, J=2.2&11 Hz, 1H), 8.31-8.10 (m, 2H), 7.97 (t, J=8.3 Hz, 1H), 7.54 (s, 1H), 5.09 (bs, 1H), 4.44 (d, J=4.3 Hz, 2H).

Preparation 40:

### 4-(tert-Butyl-dimethyl-silanyloxymethyl)-1-(2-fluoro-4-nitro-phenyl)-1H-imidazole

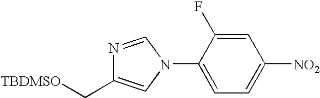

To an ice cooled solution of [1-(2-fluoro-4-nitrophenyl)-1H-imidazol-4-yl]-methanol (1.00 grams, 4.21 mmol), obtained in preparation 39, and imidazole (574 mg, 8.42 mmol) in DMF (10 mL) was added tert-butyldimethylsilyl (TBDMS) chloride (953 mg, 6.33 mmol). The reaction mixture was stirred at room temperature for 18 hours. Evaporation of solvent under reduced pressure and purification of the resulting residue by column chromatography (pet ether/ethyl acetate, 70:30) afforded-the title compound as a light brown solid (890 mg, 60%).

¹H NMR (DMSO-d₆): δ 8.34 (dd, J=10.9 Hz, 1H), 8.20-8.10 (m, 2H), 7.90 (t, J=8.3 Hz, 1H), 7.48 (s, 1H), 4.50 (s, 2H), 0.80 (s, 9H), 0.09 (s, 6H); MS (m/e): 352 (M⁺+1), 294; IR (KBr, cm⁻¹): 3436, 3126, 2931, 2859, 1537.

Preparation 41:

### 4-(tert-Butyl-dimethyl-silanyloxymethyl)-1-(2-fluoro-4-amino-phenyl)-1H-imidazole

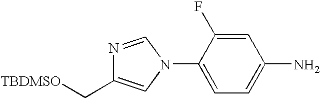

To a solution of 4-(tert-butyl-dimethyl-silanyloxymethyl)-1-(2-fluoro-4-nitro-phenyl)-1H-imidazole (800 mg, 2.28 mmol), obtained in preparation 40, in methanol (30 mL) was added NiCl₂.6H₂O (1.00 grams, 4.55 mmol) followed by the addition of sodium borohydride (259 mg, 6.83 mmol) and stirred at room temperature for 1 hours. The reaction mixture was then diluted with ethyl acetate (300 mL) and water (50 mL). The organic layer was separated, washed with brine and dried over sodium sulfate. The solvent was evaporated to get the compound as a brown thick material (585 mg, 80%).

¹H NMR (DMSO-d₆): δ 7.64 (s, 1H), 7.10-7.01 (m, 2H), 6.51-6.30 (m, 2H), 5.61 (bs, 2H), 4.51 (s, 2H), 0.81 (s, 9H), 0.03 (s, 6H); MS (m/e): 322 (M⁺+1), 264, 190; IR (KBr, cm⁻¹): 3344, 3213, 1637, 1530.

Preparation 42:

### [1-(4-Azido-2-fluoro-phenyl)-1H-imidazol-4-yl]-methanol

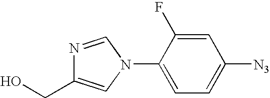

Sodium nitrite (249 mg, 3.61 mmol) was slowly added to a solution of 4-(tert-butyl-dimethyl-silanyloxymethyl)-1-(2-fluoro-4-amino-phenyl)-1H-imidazole (580 mg, 1.80 mmol), obtained in preparation 41, in 2N HCl (10 mL) at 0° C. and stirred for 1 hours. An aqueous solution containing NaN₃ (234 mg, 3.61 mmol) and NaOAc (2.60 grams, 31.1 mmol) was added to the reaction mixture. The reaction mixture was diluted with EtOAc (300 mL), The organic phase was washed with water (2×100 mL) followed by brine and dried over sodium sulfate. Removal of volatiles and purification of the resulting residue by column chromatography (chloroform/methanol, 96:4) afforded the title compound as light brown solid (315 mg, 75%).

¹H NMR (CDCl₃): δ 7.78 (bs, 1H), 7.36 (t, J=8.2 Hz, 2H), 6.94 (d, J=9.6 Hz, 2H), 4.69 (s, 2H), 3.00 (bs, 1H); MS (m/e): 234 (M⁺+1), 206; IR (KBr, cm⁻¹): 3215, 2923, 2122, 1521.

Preparation 43:

### 1-(4-Azido-2-fluoro-phenyl)-1H-imidazol-4-carbaldehyde

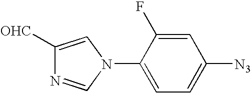

To a solution of [1-(4-azido-2-fluoro-phenyl)-1H-imidazol-4-yl]-methanol (500 mg, 2.14 mmol), obtained in preparation 42, in DCM (10 mL) was added Dess Martin Reagent (1.2 grams, 3.2 mmol) and stirred for 15 hours. Reaction mixture was diluted with ethyl acetate and the resulting solution was washed with water followed by brine and dried over sodium sulfate. Removal of volatiles and column chromatographic purification of the resulting residue (silica gel, methanol/chloroform) produced the title compound as off white solid (400 mg, 80%).

IR (KBr, cm⁻¹): 3433, 2123. 1703, 1541, 1521.

¹H NMR (DMSO-d₆): δ 9.83 (s, 1H), 8.45 (s, 1H), 8.24 (s, 1H), 7.70 (t, J=8.5 Hz, 1H), 7.42 (d, J=11.5 Hz, 1H), 7.18 (d, J=8.6 Hz, 1H).

MS (m/e): 232 (M⁺+1).

Preparation 44:

### 1-(4-Azido-2-fluoro-phenyl)-1H-imidazol-4-carbaldehyde oxime

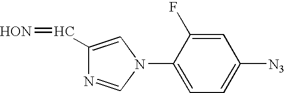

A mixture of 1-(4-azido-2-fluoro-phenyl)-1H-imidazol-4-carbaldehyde (400 mg, 1.73 mmol), obtained in preparation 43, hydroxyl amine hydrochloride (179 mg, 2.59 mmol) and pyridine (205 mg, 2.59 mmol) in methanol (15 mL) was refluxed for 2 hours. The reaction mixture was diluted with ethyl acetate and the resulting solution was washed with water followed by brine and dried over sodium sulfate. Removal of volatiles and column chromatographic purification of the resulting residue yielded the title compound (340 mg, 80%).

IR (KBr, cm⁻¹): 3067, 2855. 2117, 1520.

¹H NMR (DMSO-d₆): δ 11.66 (s, 1H), 8.11 (d, J=5.9 Hz, 2H), 7.60-7.70 (m, 1H), 7.46 (s, 1H), 7.40 (d, J=1.9 Hz, 1H), 7.13 (d, J=8.6 Hz, 1H).

MS (m/e): 247 (M⁺+1), 229.

Preparation 45:

### 1-(4-Azido-2-fluoro-phenyl)-1H-imidazole-4-carbonitrile

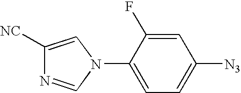

To a solution of 1-(4-azido-2-fluoro-phenyl)-1H-imidazol-4-carbaldehyde oxime (340 mg, 1.38 mmol), obtained in preparation 44, in THF was added (methoxycarbonylsulfamoyl)triethylammonium hydroxide, inner salt (Burgess Reagent, 395 mg, 1.65 mmol) and refluxed for 3 hours. Evaporation of solvent and purification of the resulting residue by column chromatographic purification produced the title compound (236 mg, 75%).

IR (KBr, cm⁻¹): 2232, 2119, 1516.

¹H NMR (DMSO-d₆): δ 8.57 (s, 1H), 8.29 (s, 1H), 7.73 (t, J=8.6 Hz, 1H), 7.43 (d, J=13.7 Hz, 1H), 7.19 (d, J=8.6 Hz, 1H).

MS (m/e): 229 (M⁺+1), 203.

Preparation 46:

### [1-(2,6-Difluoro-4-nitro-phenyl)-1H-imidazol-4-yl]-methanol

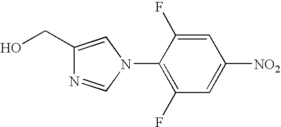

This compound was prepared by following the procedure as described in prepration 39.

IR (KBr, cm⁻¹): 3139, 3043, 1529.

¹H NMR (CDCl₃): δ 8.05 (d, J=7.5 Hz, 2H), 7.83 (s, 1H), 7.26 (s, 1H), 4.72 (s, 2H).

MS (m/e): 256 (M⁺+1), 236, 176.

Preparation 47:

### 4-(tert-Butyl-dimethyl-silanyloxymethyl)-1-(2,6-difluoro-4-nitro-phenyl)-1H-imidazole

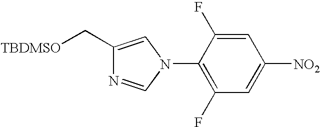

This compound was prepared by following the procedure as described in prepration 40.

IR (KBr, cm−1): 2932, 2858, 1523, 1350.

¹H NMR (CDCl₃): δ 8.04 (d, J=7.3 Hz, 2H), 7.77 (s, 1H), 7.17 (s, 1H), 4.78 (s, 2H), 0.9 (s, 9H), 0.13 (s, 6H).

MS (m/e): 370 (M⁺+1), 312, 238.

Preparation 48:

### 4-[4-(tert-Butyl-dimethyl-silanyloxymethyl)-imidazol-1-yl]-3,5-difluoro-phenylamine

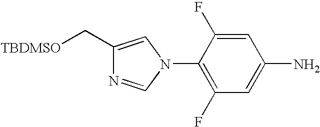

This compound was prepared by following the procedure as described in prepration 41.

IR (Neat, cm¹): 3354, 2929, 1651, 1538.

¹H NMR (CDCl₃): δ 7.54 (s, 1H), 6.98 (s, 1H), 6.39 (d, J=9.8 Hz, 2H), 4.78 (s, 3H), 0.95 (s, 9H), 0.13 (s, 6H).

MS (m/e): 340 (M⁺+1), 282, 208.

Preparation 49:

### [1-(4-Azido-2,6-difluoro-phenyl)-1H-imidazol-4-yl]-methanol

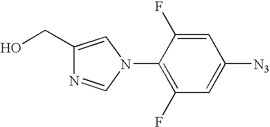

This compound is prepared by following the procedure as described in prepration 42.

IR (KBr, cm⁻¹): 3156, 2112.

¹H NMR (CDCl₃): δ 7.66 (s, 1H), 7.09 (s, 1H), 6.78(d, J=8.5 Hz, 2H), 4.69 (s, 2H).

MS (m/e): 252 (M++1), 226.

Preparation 50:

### 1-(4-Azido-2,6-difluoro-phenyl)-1H-imidazol-4-carbaldehyde

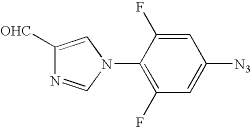

This compound was prepared by following the procedure as described in prepration 43.

IR (KBr, cm⁻¹): 2191, 2127, 1699.

¹H NMR (CDCl₃): δ 9.82 (s, 1H), 8.41 (s, 1H), 8.19 (s, 1H), 7.35(d, J=8.6 Hz, 2H).

MS (m/e): 250 (M⁺+1), 224.

Preparation 51:

### 1-(4-Azido-2,6-difluoro-phenyl)-1H-imidazol-4-carbaldehyde oxime

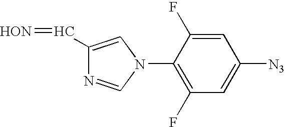

This compound was prepared by following the procedure as described in prepration 44.

IR (KBr, cm⁻¹): 3433, 3184, 3010, 2858, 2119, 1528.

¹H NMR (CDCl₃): δ 11.66 & 10.98 (2s in a ratio of 3:1, 1H), 8.05(d, J=12 Hz, 2H), 7.40 (s, 1H), 7.30(d, J=8.9 Hz, 2H).

MS (m/e): 265 (M⁺+1), 247, 221.

Preparation 52:

### 1-(4-Azido-2,6-difluoro-phenyl)-1H-imidazole-4-carbonitrile

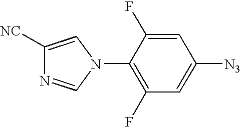

This compound was prepared by following the procedure as described in prepration 45.

IR (KBr, cm⁻¹): 3421, 2925, 2133, 1521.

¹H NMR (CDCl₃): δ 8.54 (s, 1H), 8.27 (s, 1H), 7.31 (d, J=8.9 Hz, 2H).

MS (m/e): 247 (M⁺+1), 218, 166, 126.

Preparation 53:

### {1-[4-(1,3-Dioxo-1,3-dihydro-isoindol-2-yl)-3-fluoro-phenyl]-1H-[1,2,3]triazol-4-yl-methyl}-thiocarbamic acid O-methyl ester

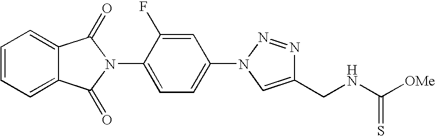

To a DMF solution (2 mL) of 2-(4-azido-2-fluoro-phenyl)-isoindole-1,3-dione (142 mg, 0.58 mmol), obtained in preparation 10, and diisopropylethyl amine (75 mg, 0.58 mmol) was added prop-2-ynyl-thiocarbamic acid O-methyl ester (100 mg, 0.78 mmol) followed by the addition of cuprous iodide (196 mg, 1.03 mmol) in portion and stirred at room temperature for 0.5 hours. Saturated solution of ammonium chloride (5 mL) was added to the reaction mixture followed by the addition of two drops of ammonium hydroxide solution. The reaction mixture was then diluted with ethyl acetate (50 mL) and aqueous layer was separated. The organic phase was washed with water followed by brine and dried over sodium sulfate. Evaporation of volatiles on rotavapor and purification of the resulting residue through silica gel column (ethyl acetate/pet ether, 1:1) yielded the title compound (124 mg, 60%).

¹H NMR (CDCl₃): δ 8.17 (s, 1H), 8.02-7.93 (m, 2H), 7.90-7.62 (m, 4H), 7.55 (t, J=7.5 Hz, 1H), 6.92 (bs, 1H), 4.93 & 4.72 (2 d, J=5.9 Hz, 2H, rotamers in a ratio of 4:1), 4.13 & 4.01 (2s, 3H, rotamers in a ratio of 1:4); MS (m/e): 412 (M⁺+1), 380, 323, 257; IR (KBr, cm⁻¹): 1731, 1528, 1384.

Preparation 54:

### [1-(4-Amino-3-fluoro-phenyl)-1H-[1,2,3]triazol-4-ylmethyl]-thiocarbamic acid O-methyl ester

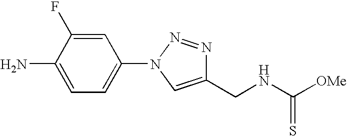

To a solution of {1-[4-(1,3-dioxo-1,3-dihydro-isoindol-2-yl)-3-fluoro-phenyl]-1H-[1,2,3]triazol-4-yl-methyl}-thiocarbamic acid O-methyl ester (75 mg, 0.182 mmol), obtained in preparation 53, in methanol (3 mL) was added ethylene diamine (60 mg, 1.08 mmol) and stirred at room temperature for 3 hours. Removal of methanol on rotavapor and purification of the resulting residue through a silica gel column (ethyl acetate/pet ether, 3:1) yielded the title compound as brown colour solid (46 mg, 91%).

¹H NMR (CDCl₃): δ 7.91 & 7.69 (2s, rotamers in a ratio of 4:1, 1H), 7.33 (d, J=11.3 Hz, 1H), 7.17 (bs, 1H), 6.99 (bs, 1H), 6.79 (t, J=8.9 Hz, 1H), 4.82 & 4.57 (2 d, J=5.7 Hz, 2H, rotamers in a ratio of 4:1), 4.04 & 3.92 (2s, 3H, rotamers in a ratio of 1:4); MS (m/e): 282 (M⁺+1), 250.

Preparation 55:

### {1-[4-(1,3-Dioxo-1,3-dihydro-isoindol-2-yl)-3,5-difluoro-phenyl]-1H-[1,2,3]triazol-4-ylmethyl}-thiocarbamic acid O-methyl ester

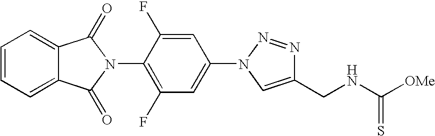

To a DMF solution (2 mL) of 2-(4-azido-2,6-difluorophenyl)-isoindole-1,3-dione (1 g, 3 mmol), obtained in preparation 18, and diisopropylethyl amine (388.5 mg, 3 mmol) was added prop-2-ynyl-thiocarbamic acid O-methyl ester (580 mg, 4.5 mmol) followed by the addition of cuprous iodide (1.14 grams, 6 mmol) in portion and stirred at room temperature for 0.5 hours. Saturated solution of ammonium chloride (5 mL) was added to the reaction mixture followed by the addition of few drops of ammonium hydroxide solution. The solution was then diluted with ethyl acetate (500 mL) and aqueous layer was separated. The organic layer was washed with water followed by brine and dried over sodium sulfate. Evaporation of volatiles on rotavapor and purification of the resulting residue through silica gel column (ethyl acetate/pet ether: 1:4) yielded the title compound (663 mg, 76%).

¹H NMR (CDCl₃): δ 8.18 (s, 1H), 8.05-7.96 (m, 2H), 7.91-7.77 (m, 2H), 7.58 (d J=7.8 Hz, 2H), 6.88 (bs, 1H), 4.94 & 4.72 (2 d, J=5.8 Hz, 2H, rotamers in a ratio of 4:1), 4.13 & 4.01 (2 s, 3H, rotamers in a ratio of 1:4); MS (m/e): 430 (M⁺+1), 398, 311, 275; IR (KBr, cm⁻¹): 3438, 2924, 2854, 1739.

### EXAMPLE 1

### [1-(4-Pyrrol-1-yl-phenyl)-1H-[1,2,3]triazol-4-ylmethyl]-thiocarbamic acid O-methyl ester

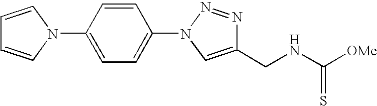

To a solution of [1-(4-amino-phenyl)-1H-[1,2,3]triazol-4-ylmethyl]-thiocarbamic acid O-methyl ester (0.10 grams, 0.4 mmol), obtained in preparation 7, in glacial acetic acid (10 mL) was added 2,5-dimethoxy tetrahydrofuran (0.06 grams, 0.42 mmol) and heated to 80° C. for 0.5 hours. The reaction mixture was then diluted with ethyl acetate (50 mL) and the organic phase was washed with water followed by brine and dried over sodium sulfate. Evaporation of volatiles on rotavapor and purification of the resulting residue through a silica gel column (ethyl acetate/chloroform, 1:4) yielded the title compound as white solid (70 mg, 60%). mp 150° C.

¹HNMR (CDCl₃): δ 8.13 (s, 1H), 7.80 (d, J=8.6 Hz, 2H), 7.55 (d, J=8.6 Hz, 2H), 7.12 (m, 1H), 7.01 (bs, 1H, D₂O exchangeable), 6.44 (m, 1H), 4.91 & 4.78 (2 d, J=5.9 Hz, 2H, rotamers in a ratio of 4:1), 4.15 & 4.09 (2 s, 3H, rotamers in a ratio of 1:4); MS (m/e): 314 (M³⁰ +1).

### EXAMPLE 2

### {1-[4-(3-Formyl-pyrrol-1-yl)-phenyl]-1H-[1,2,3]triazol-4-ylmethyl}-thiocarbamic acid O methyl ester

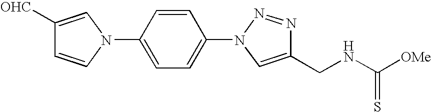

To a solution of [1-(4-aminophenyl)-1H-[1,2,3]triazol-4-ylmethyl]-thiocarbamic acid O-methyl ester (0.10 grams, 0.4 mmol), obtained in preparation 7, in glacial acetic acid (10 mL) was added 2,5-dimethoxy tetrahydrofuran-3-carboxaldehyde (0.06 g, 0.42 mmol) and heated to 80° C. for 0.5 hours. The reaction mixture was diluted with ethyl acetate (20 mL) and the organic layer was washed with water followed by brine and dried over sodium sulfate. Evaporation of volatiles on rotavapor and purification of the resulting residue through a silica gel column (ethyl acetate/chloroform, 1:4) yielded the title compound as white solid (70 mg, 53%). mp 160-162° C.

¹HNMR (CDCl₃): δ 9.91 (s, 1H), 9.23 (bs, 1H, D₂O exchangeable), 8.40 (s, 1H), 7.95 (d, J=8.8 Hz, 2H), 7.72 (d, J=8.8 Hz, 2H), 7.30 (m, 1H), 6.84 (s, 1H), 4.91 & 4.65 (2 d, J=5.9 Hz, 2H, rotamers in a ratio of 4:1), 4.03 & 3.91 (2 s, 3H, rotamers in a ratio of 1:4); MS (m/e): 342 (M⁺+1), 310.

### EXAMPLE 3

### {1-[4-(3-Hydroxymethyl-pyrrol-1-yl)-phenyl]-1H-[1,2,3]triazol-4-ylmethyl}-thiocarbamic acid O-methyl ester

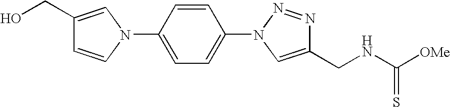

Sodium borohydride (11 mg, 0.28 mmol) was added to a solution of {1-[4-(3-formyl-pyrrol-1-yl)-phenyl]-1H-[1,2,3]triazol-4-ylmethyl}-thiocarbamic acid O-methyl ester (30 mg, 0.09 mmol), obtained in example 2, in methanol (4 mL) at 0° C. and stirred for 3 hours. Methanol was removed on a rotavapor and the crude residue was purified through a silica gel column (ethyl acetate/pet ether, 1:1) to yield the title compound as light yellow solid (28 mg, 93%).

¹HNMR (CDCl₃+DMSO-d₆): δ 9.46 (bs, 1H), 8.49 (s, 1H), 8.02 (s, 1H), 7.92 (d, J=8.3 Hz, 2H), 7.64 (d, J=8.3 Hz, 2H), 7.23 (s, 1H), 6.34 (s, 1H), 4.82 & 4.70 (2 d, J=5.4 Hz, 2H, rotamers in a ratio of 4:1), 4.58-4.45 (m, 2H), 4.03 & 3.96 (2 s, 3H, rotamers in a ratio of 1:4); MS (m/e): 344 (M⁺+1), 312, 294; IR (KBr, cm⁻¹): 3356, 2924, 1676.

### EXAMPLE 4

### [1-(3-Fluoro-4-pyrrol-1-yl-phenyl)-1H-[1,2,3]triazol-4-ylmethyl]-thiocarbamic acid O-methyl ester

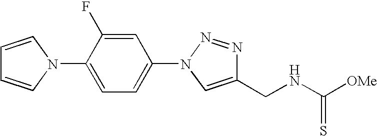

To a DMF solution (2 mL) of 1-(4-azido-2-fluorophenyl)-1H-pyrrole (82 mg, 0.41 mmol), obtained in preparation 12, and diisopropylethyl amine (53 mg, 0.41 mmol) was added prop-2-ynyl-thiocarbamic acid O-methyl ester (82 mg, 0.61 mmol) followed by the addition of cuprous iodide (154 mg, 0.81 mmol) in portion and stirred at room temperature for 0.5 hours. Saturated solution of ammonium chloride (5 mL) was added to the reaction mixture followed by the addition of two drops of ammonium hydroxide solution. The reaction mixture was then diluted with ethyl acetate (50 mL) and aqueous layer was separated. The organic layer was washed with water followed by brine and dried over sodium sulfate. Evaporation of volatiles on rotavapor and purification of the resulting residue through silica gel column (ethyl acetate/pet ether, 1:2) yielded the title compound as light brown solid (34 mg, 25%).

¹H NMR (CDCl₃): δ 8.14 (s, 1H), 7.68 (d, J=12.2 Hz, 1H), 7.50 (bs, 2H), 7.08 (s, 2H), 6.94 (bs, 1H), 6.40 (s, 2H), 4.92 & 4.65 (2 d, J=5.9 Hz, 2H, rotamers in a ratio of 4:1), 4.12 & 4.00 (2 s, 3H, rotamers in a ratio of 1:4); MS (m/e): 332 (M⁺+1), 300, 213; IR (KBr, cm⁻): 3427, 2925, 2854, 1534.

### EXAMPLE 5

### {1-[4-(3-Cyano-pyrrol-1-yl)-3-flouro-phenyl]-1H-[1,2,3]triazol-4-ylmethyl}-thiocarbamic acid O-methyl ester

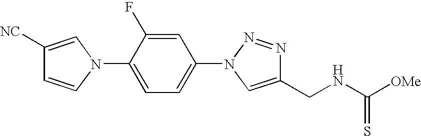

To a DMF solution (2 mL) of 1-(4-azido-2-fluoro-phenyl)-1H-pyrrole-3-carbonitrile (60 mg, 0.26 mmol), obtained in preparation 15, was added prop-2-ynyl-thiocarbamic acid O-methyl ester (44 mg, 0.34 mmol) followed by the addition of cuprous iodide (100 mg, 0.53 mmol) in portion and stirred at room temperature for 0.5 hours. Saturated solution of ammonium chloride (5 mL) was added to the reaction mixture followed by the addition of few drops of ammonium hydroxide solution. The reaction mixture was then diluted with ethyl acetate (200 mL) and aqueous layer was separated. The organic phase was washed with water followed by brine and dried over sodium sulfate. Evaporation of volatiles on rotavapor and purification of the resulting residue through silica gel column (ethyl acetate/pet ether: 1:1) yielded the title compound (56 mg, 61%).

¹H NMR (CDCl₃): δ 8.19 & 7.96 (2 s, 1H, rotamers in a ratio of 4:1 ), 7.78 (d, J=11.2 Hz, 1H), 7.85-7.52 (m, 4H),7.03 (s, 1H), 6.92 (bs, 1H), 6.66 (s, 1H), 4.93 & 4.7 J=5.8 Hz, 2H, rotamers in a ratio of 4:1), 4.13 & 4.01 (2 s, 3H, rotamers in a ratio of 1:4); MS (m/e):357 (M⁺+1), 325, 238; R (KBr, cm⁻¹): 3346, 2234, 1537.

### EXAMPLE 6

### {1-[3-Fluoro-4-(3-formyl-pyrrol-1-yl)-phenyl]-1H-[1,2,3]triazol-4-ylmethyl}-thiocarbamic acid O-methyl ester

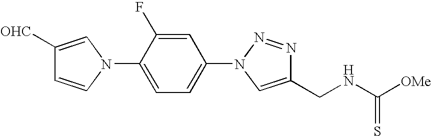

To a solution of [1-(4-amino-3-fluoro-phenyl)-1H-[1,2,3]triazol-4-ylmethyl]-thiocarbamic acid O-methyl ester (123 mg, 0.43 mmol), obtained in preparation 54, in glacial acetic acid (4 mL) was added 2,5-dimethoxy tetrahydrofuran-3-carboxaldehyde (64 mg, 0.48 mmol) and heated to 114° C. for 3 hours. The reaction mixture was diluted with water (20 mL) and neutralized with saturated solution of sodium bicarbonate. The aqueous layer was then extracted with ethyl acetate (50 mL×2) and the combined extracts were washed with water followed by brine. After drying over sodium sulfate, ethyl acetate was removed on rotavapor and the resulting residue was purified through a silica gel column (ethyl acetate/pet ether: 2:1) to yield the title compound as light brown solid (79 mg, 50%).

¹HNMR (CDCl₃): δ 9.87 (s, 1H), 8.17 & 7.95 (2 s, 1H, rotamers in a ratio of 4:1), 7.76 (d, J=11.2 Hz, 1H), 7.65-7.51 (m, 3H), 7.06 (s, 1H), 6.92 (bs, 1H), 4.92 & 4.67 2 d, J=5.9 Hz, 2H, rotamers in a ratio of 4:1), 4.11 & 3.99 (2 s, 3H, rotamers in a ratio of 1:4); MS (m/e): 360 (M⁺+1), 328, 298; IR (KBr, cm⁻¹): 3356, 2924, 1676.

### EXAMPLE 7

### [1-(3,5-difluoro-4-pyrrol-1-yl-phenyl)-1H-[1,2,3]triazol-4-ylmethyl]-thiocarbamic acid O-methyl ester

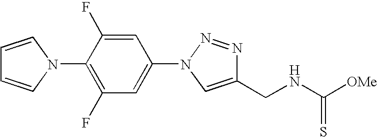

To a solution of [1-(4-amino-3,5-difluoro-phenyl)-1H-[1,2,3]triazol-4-ylmethyl]-thiocarbamic acid O-methyl ester (180 mg, 0.6 mmol), obtained in preparation 37, in glacial acetic acid (3 mL) was added 2,5-dimethoxy tetrahydrofuran (79.23 mg, 0.6 mmol) and heated to 100° C. for 4 hours. The reaction mixture was diluted with water (20 mL) and neutralized with saturated solution of sodium bicarbonate. The aqueous layer was then extracted with ethyl acetate (50 mL×2) and the combined extracts were washed with water followed by brine. After drying over sodium sulfate, ethyl acetate was removed on rotavapor and the resulting residue was purified by passing through a silica gel column (ethyl acetate/pet ether: 1:1) to yield the title compound as light brown solid (169 mg, 81%).

¹HNMR (CDCl₃): δ 8.16 & 7.96 (2 s, 1H, rotamers in a ratio of 4:1), 7.52 (d, J=8.3 Hz, 2H), 6.95 (s, 3H), 6.41 (s, 2H), 4.93 & 4.78 (2 d, J=5.9, 2H, rotamers in a ratio of 4:1), 4.13 & 4.01 (2 s, 3H, rotamers in a ratio of 1:4); MS (m/e): 350 (M⁺+1), 321, 306, 231; IR (KBr, cm⁻¹): 3315, 2926, 1538, 1035.

### EXAMPLE 8

### {1-[4-(3-Cyano-pyrrol-1-yl)-3,5-difluoro-phenyl]-1H-[1,2,3]triazol-4-ylmethyl}-thiocarbamic acid O-methyl ester

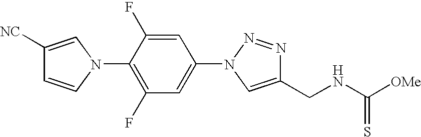

To a DMF solution (2 mL) of 1-(4-azido-2,6-difluoro-phenyl)-1H-pyrrole-3-carbonitrile (166 mg, 0.68 mmol), obtained in preparation 22, and diisopropylethyl amine (88 mg, 0.88 mmol) was added prop-2-ynyl-thiocarbamic acid O-methyl ester (131 mg, 1.02 mmol) followed by the addition of cuprous iodide (257 mg, 1.36 mmol) in portion and stirred at room temperature for 4 hours. Saturated solution of ammonium chloride (5 mL) was added to the reaction mixture followed by the addition of few drops of ammonium hydroxide solution. The reaction mixture was then diluted with ethyl acetate (200 mL) and aqueous layer was separated. The organic phase was washed with water followed by brine and dried over sodium sulfate. Evaporation of volatiles on rotavapor and purification of the resulting residue through silica gel column (ethyl acetate/pet ether: 1:2) yielded the title compound; (56 mg, 22%).

¹H NMR (CDCl₃): δ 8.17 & 7.91(2 s, 1H, rotamers in a ratio of 4:1), 7.58 (d, J=8.3 Hz, 2H), 7.39 (s, 1H), 6.90 (bs, 2H), 6.64 (s, 1H), 4.91 & 4.66 (2 d, J=5.8 Hz, 2H, rotamers in a ratio of 4:1), 4.10 & 3.98 (2 s, 3H, rotamers in a ratio of 1:4); MS (m/e):375 (M⁺+1), 343, 256, 181; IR (KBr, cm⁻¹): 3239, 2924, 2226, 1543, 1044.

### EXAMPLE 9

### [1-(3,5-Difluoro-4-imidazol-1-yl-phenyl)-1H-[1,2,3]triazol-4-ylmethyl]-thiocarbamic acid O-methyl ester

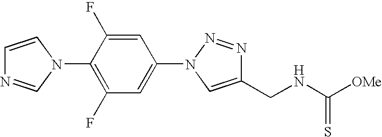

To a DMF solution (5 mL) of 1-(4-azido-2,6-difluorophenyl)-1H-imidazole (500 mg, 2.26 mmol), obtained in preparation 25, and-diisopropylethyl amine (291 mg, 2.26 mmol) was added prop-2-ynyl-thiocarbamic acid O-methyl ester (321 mg, 2.48 mmol) followed by the addition of cuprous iodide (859 mg, 4.52 mmol) in portion and stirred at room temperature for 0.5 hours. Saturated solution of ammonium chloride (20 mL) was added to the reaction mixture followed by the addition of few drops of ammonium hydroxide solution. The blue colour solution was diluted with ethyl acetate (100 mL) and aqueous layer was separated. The organic layer was washed with water followed by brine and dried over sodium sulfate. Evaporation of volatiles on rotavapor and purification of the resulting residue through silica gel column yielded the pink color compound (220 mg, 28%). mp 180° C.

¹H NMR (CDCl₃): δ 9.71 (s, 1H), 8.89 & 8.85 (2 s, 1H, rotamers in 4:1 ratio), 8.13 (d, J=9.5 Hz, 2H), 8.02 (s, 1H), 7.53 (s, 1H), 7.12 (s, 1H), 4.77 & 4.48 (2 d, J=5.4 Hz, 2H, rotamers in 4:1 ratio), 3.96 & 3.90 (2 s, 3H, rotamers 1:4 ratio); MS (m/e): 351 (M⁺+1), 319; IR (KBr, cm⁻¹): 3443, 1535.

### EXAMPLE 10

### [1-(3-Fluoro-4-imidazol-1-yl-phenyl)-1H-[1,2,3]triazol-4-ylmethyl]-thiocarbamic acid O-methyl ester

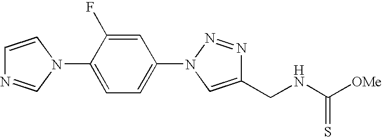

To a DMF solution (5 mL) of 1-(4-azido-2-fluorophenyl)-1H-imidazole (500 mg, 2.46 mmol), obtained in preparation 28, and diisopropylethyl amine (317 mg, 2.46 mmol) was added prop-2-ynyl-thiocarbamic acid O-methyl ester (349 mg, 2.7 mmol) followed by the slow addition of cuprous iodide (934 mg, 4.92 mmol) and stirred at room temperature for 0.5 hours. Saturated solution of ammonium chloride (20 mL) was added to the reaction mixture followed by the addition of few drops of ammonium hydroxide solution. The blue colour solution was diluted with ethyl acetate (100 mL) and aqueous layer was separated. The organic layer was washed with water followed by brine and dried over sodium sulfate. Evaporation of volatiles on rotavapor and purification of the residue by silica gel column chromatography yielded the pink color compound (245 mg, 30%). mp 165-167° C.

¹H NMR (CDCl₃+DMSO-d₆): δ 9.01 (bs, 1H), 8.40 & 8.28 (2 s, 1H, rotamers in 4:1 ratio), 7.99 (s, 1H), 7.90 (dd, J=2.1 & 11.3 Hz, 1H), 7.80-7.60 (m, 2H), 7.39 (s, 1H), 7.25 (s, 1H), 4.87 & 4.62 (2 d, J=5.6 Hz, 2H, rotamers in 4:1 ratio), 4.06 & 3.99 (2 s, 3H, rotamers in 1:4 ratio); MS (m/e): 332 (M⁺+1), 301, 246, 214; IR (KBr, cm⁻¹): 3431, 1535.

### EXAMPLE 11

### 4-Azidomethyl-1-(3-fluoro-4-pyrazol-1-yl-phenyl)-1H-[1,2,3]triazole

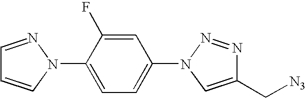

Sodium azide (0.98 grams, 15 mmol) was added to a mixture of methanesulfonic acid [1-(3-fluoro-4-pyrazol-1-ylphenyl)-1H-[1,2,3]triazol-4-yl]-methyl ester & methanesulfonic acid [1-(3-fluoro-4-pyrazol-1-ylphenyl)-1H-[1,2,3]triazol-5-yl]-methyl ester (1.70 grams, 5 mmol), obtained in preparation 33, in DMF (15 mL) and heated to 90° C. for 1 h. The reaction mixture was diluted with water (50 mL) and extracted with ethyl acetate (50 mL×3). The combined extracts were washed with water followed by brine and dried over sodium sulfate. Evaporation of volatiles and purification of the resulting residue by column chromatography yielded the required regioisomer as pale yellow solid (800 mg, 55%).

¹H NMR (DMSO-d₆): δ 8.99 (s, 1H), 8.32 (s, 1H), 8.24 (dd, J=2.3 & 20.2 Hz, 1H), 8.11-7.93 (m, 2H), 7.86 (s, 1H), 6.65 (s, 1H), 4.61 (s, 2H); MS (m/e): 285 M⁺+1); IR (KBr, cm⁻¹): 3426, 2083, 1533.

### EXAMPLE 12

### C-[1-(3-Fluoro-4-pyrazol-1-yl-phenyl)-1H-[1,2,3]triazol-4-yl]methylamine

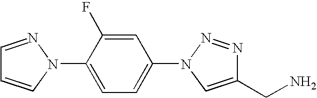

A mixture of 4-azidomethyl-1-(3-fluoro-4-pyrazol-1-yl-phenyl)-1H-[1,2,3]triazole (800 mg, 3.10 mmol), obtained in example 11, and triphenylphosphine (894 mg, 3.40 mmol) in THF (10 mL) was stirred at room temperature for 3 hours. It was then warmed to 40° C. after the addition of 4-5 drops of water and allowed to stir at the same temperature for 16 hours. The reaction mixture was diluted with water and extracted with ethyl acetate (50 mL×2). Combined ethyl acetate extracts were washed with water followed by brine and dried over sodium sulfate. Evaporation of volatiles and purification of the resulting residue by column chromatography with silica gel (60-120 mesh) yielded the title compound as pale yellow solid (580 mg, 80%).

¹H NMR (DMSO-d₆): δ 8.72 (s, 1H), 8.30 (s, 1H), 8.20-7.86 (m, 4H), 6.67 (s, 1H), 3.81 (s, 2H), 2.85 (bs, 2H); MS (m/e): 259 (M⁺+1); IR (KBr, cm⁻¹): 3382, 3156, 3114, 2923, 1536.

### EXAMPLE 13

### [1-(3-Fluoro-4-pyrazol-1-yl-phenyl)-1H-[1,2,3]triazol-4-yl-methyl]-thiocarbamic acid O-methyl ester

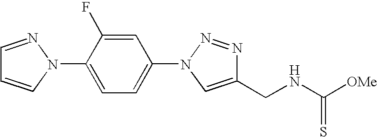

To a mixture of C-[1-(3-fluoro-4-pyrazol-1-yl-phenyl)-1H-[1,2,3]triazol-4-yl]-methylamine (300 mg, 1.16 mmol), obtained in example 12, in CHCl₃ (40 mL) and saturated sodium bicarbonate solution (5 mL), was added thiophosgene (133 mg, 1.16 mmol) and stirred at room temperature for 0.5 hours. The reaction mixture was diluted with ethyl acetate (50 mL) and the organic layer was washed with water followed by brine and dried over sodium sulfate. Evaporation of volatiles left a residue, which was refluxed with methanol (30 mL) for 16 hours. Removal of methanol on rotavapor and purification of the resulting residue through a silica gel column (60-120 mesh) yielded the title compound as white solid (180 mg, 46%).

¹H NMR (CDCl₃): δ 8.22-7.95 (m, 3H), 7.81-7.52 (m, 3H), 6.97 (bs, 1H), 6.53 (s, 1H), 4.92 & 4.69 (2d, J=4.8 Hz, rotamers in the ratio of 1:4, 2H)), 4.15 & 4.03 (2s, rotamers in the ration of 1:4, 3H); MS (m/e): 333, 301, 214 (M⁺+1); IR (KBr, cm⁻¹): 3187, 3023, 2933, 1535.

### EXAMPLE 14

### N-[1-(3-Fluoro-4-pyrazol-1-yl-phenyl)-1H-[1,2,3]triazol-4-yl-methyl]acetamide

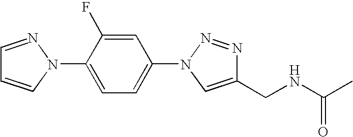

A solution of 4-azidomethyl-1-[3-fluoro-4-pyrazol-1-yl-phenyl]-1H-[1,2,3]triazole (500 mg), obtained in example 11, in thiolacetic acid (1 mL) was stirred for 12 hours at room temperature. The reaction mixture was then absorbed on silica gel and purified by column chromatography (over silica gel) to yield the title compound as off white solid (350 mg, 66%).

¹H NMR (DMSO-d₆): δ 8.20-8.09 (m, 3H), 7.81-7.78 (m, 2H), 7.62 (d, J=8.7 Hz, 1H), 6.53 (s, 1H), 6.28 (bs, 1H), 2.03 (s, 3H); MS (m/e): 301 (M⁺+1); IR (KBr, cm⁻¹): 3303, 3125, 3080, 1659 and 1537.

### EXAMPLE 15

### N-[1-(3-Fluoro-4-pyrazol-1-yl-phenyl)-1H-[1,2,3]triazol-4-yl-methyl]-thioacetamide

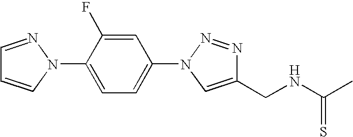

To a solution of N-[1-(3-fluoro-4-pyrazol-1-yl-phenyl)-1H-[1,2,3]triazol-4-yl-methyl]-acetamide (150 mg, 0.50 mmol), obtained in example 14, in 1,4-dioxane (5 mL) was added Lawessen's reagent (242 mg, 0.60 mmol) and refluxed for 12 hours. The reaction mixture was diluted with ethyl acetate (50 mL) and the organic portion was washed with water followed by brine and dried over sodium sulfate. Evaporation of volatiles left a residue, which was purified by column chromatography (over silica gel) to yield the title compound as off white solid (100 mg, 63%).

¹H NMR (CDCl₃): δ 8.42 (bs, 1H), 8.30-8.11 (m, 3H), 7.82-7.55 (m, 3H), 6.57 (s, 1H), 5.05 (d, J=5.6 Hz, 2H), 2.61(s, 3H); MS (m/e): 317 (M⁺+1); IR (KBr, cm⁻¹): 3220, 3019, 1536.

### EXAMPLE 16

### 1-Ethyl-3-[1-(3-fluoro-4-pyrazol-1-yl-phenyl)-1H-[1,2,3]triazol-4-yl-methyl]-thiourea

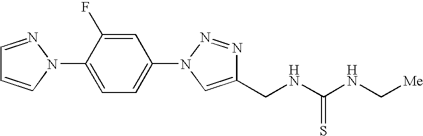

To a solution of C-[1-(3-fluoro-4-pyrazol-1-yl-phenyl)-1H-[1,2,3]triazol-4-yl]-methylamine (200 mg, 0.77 mmol), obtained in example 12, in dichloromethane (10 mL) was added triethylamine (172 mg, 1.70 mmol) followed by the addition of ethylisothiocyanate (135 mg, 1.55 mmol) at 0° C. and stirred at room temperature for 18 hours. The reaction mixture was diluted with ethyl acetate (100 mL) and washed with water followed by brine and dried over sodium sulfate. Evaporation of volatiles on rotavapor and purification of the resulting residue by column chromatography (over silica gel) yielded the title compound as pale yellow solid (110 mg, 41%).

¹H NMR (DMSO-d₆): δ 8.78 (s, 1H), 8.32-7.81 (m, 6H), 7.57 (bs, 1H), 6.61 (s, 1H), 4.78 (d, J=4.9 Hz, 2H), 3.38 (bs, 2H, after D₂O exchange), 1.0 (t, J=7.1 Hz, 3H); MS (m/e): 346 (M⁺+1), 317, 301, 259, 214; IR (KBr, cm⁻¹): 3330, 1532, 1396, 1043.

### EXAMPLE 17

### 1-[3-Fluoro-4-pyrazol-1-yl-phenyl]-1H-[1,2,3]triazol-4-yl-methylamino-hydrazino methanethione

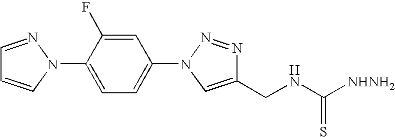

To a solution of C-[1-(3-fluoro-4-pyrazol-1-ylphenyl)-1H-[1,2,3]triazol-4-yl]-methylamine (250 mg, 0.97 mmol), obtained in example 12, in DMF (2 mL) was added 4-(4-methylphenyl)-3-thiosemicarbazide (193 mg, 1.06 mmol) and heated to 90° C. for 12 hours. The reaction mixture was then diluted with ethyl acetate (150 mL) and washed with water followed by brine and dried over sodium sulfate. Evaporation of volatiles on rotavapor and purification of the resulting residue by column chromatography (silica gel) yielded the title compound as off white powder (105 mg, 32%).

¹H NMR (DMSO-d₆): δ 8.82 (s, 1H), 8.65 (bs, 1H), 8.31 (s, 1H), 8.19-7.88 (m, 4H), 7.80 (s, 1H), 6.62 (s, 1H), 4.45 (d, J=5.6 Hz, 2H); MS (m/e): 287 (M⁺−45), 258, 229, 214, 202; IR (KBr, cm⁻¹): 3438, 3257, 1648, 1532.

### EXAMPLE 18

### N-[1-(3-Fluoro-4-pyrazol-1-yl-phenyl)-1H-[1,2,3]triazol-4-yl-methyl]-methane-sulfonamide

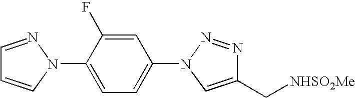

To a solution of C-[1-(3-fluoro-4-pyrazol-1-yl-phenyl)-1H-[1,2,3]triazol-4-yl]-methylamine (200 mg, 0.77 mmol), obtained in example 12, in dichloromethane (10 mL) was added triethylamine (156 mg, 1.55 mmol) followed by the addition of methanesulfonyl chloride (97 mg, 0.85 mmol) at 0° C. and stirred at the same temperature for 1 h. The reaction mixture was diluted with ethyl acetate (100 mL) and washed with water followed by brine and dried over sodium sulfate. Evaporation of volatiles under vacuum yielded the title compound as off white powder (180 mg, 69%).

¹H NMR (CDCl₃+DMSO-d₆): δ 8.51 (s, 1H), 8.15-7.70 (m, 5H), 7.63 (bs, 1H), 6.59 (s, 1H), 4.45 (s, 2H), 3.01 (s, 3H); MS (m/e): 337(M⁺+1); IR (KBr, cm⁻¹): 3446, 3237, 1536, 1308.

### EXAMPLE 19

### [1-(3-Fluoro-4-pyrazol-1-yl-phenyl]-1H-[1,2,3]triazol-4-yl]-methyl carbamic acid O-methyl ester

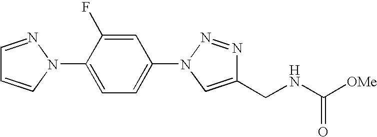

To a solution of C-[1-(3-fluoro-4-pyrazol-1-yl-phenyl)-1H-[1,2,3]triazol-4-yl]-methylamine (200 mg, 0.78 mmol), obtained in example 12, in dichloromethane (10 mL) was added N-ethyldiisopropylamine (219 mg, 1.7 mmol) followed by the addition of methyl chloroformate (87 mg, 0.92 mmol) at 0° C. and stirred at the same temperature for 2 h. The reaction mixture was then diluted with ethyl acetate (150 mL) and washed with water followed by brine and dried over sodium sulfate. Removal of volatiles under reduced pressure and purification of the resulting residue by column chromatography (over silica gel) yielded the title compound as white powder (150 mg, 61%).

¹H NMR (DMSO-d₆): δ 8.76 (s, 1H), 8.31 (s, 1H), 8.10 (d, J=12.6 Hz, 1H), 8.01-8.10 (m, 2H), 7.91 (s, 1H), 6.59 (s,1H), 4.30 (d, J=5.8 Hz, 2H), 3.57 (s, 3H); MS (m/e): 317 (M⁺+1); IR (KBr, cm⁻¹): 3366, 3128, 2926, 1724, 1536.

### EXAMPLE 20

### [1-(3-Fluoro-4-[1,2,4]triazol-1-yl-phenyl)-1H-[1,2,3]triazol-4-yl-methyl]-thiocarbamic acid O-methyl ester

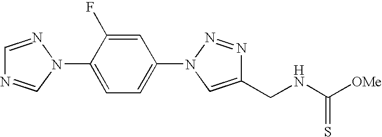

To a DMF solution (5 mL) of 1-(4-azido-2-fluorophenyl)-1H-[1,2,4]triazole (250 mg, 1.23 mmol), obtained in preparation 36, and diisopropylethyl amine (155 mg, 1.2 mmol) was added prop-2-ylnyl-thiocarbamic acid O-methyl ester (189 mg, 1.47 mmol) followed by the addition of cuprous iodide (228 mg, 1.2 mmol) in portion and stirred at room temperature for 0.5 hours. Saturated solution of ammonium chloride (10 mL) was added to the reaction mixture followed by the addition of few drops of ammonium hydroxide solution. The blue colour solution was diluted with ethyl acetate (100 mL) and aqueous layer was separated. The organic layer was washed with water followed by brine and dried over sodium sulfate. Evaporation of volatiles on rotavapor and purification of the resulting residue through silica gel column yielded the white color compound (220 mg, 32%). Mp 179° C.

¹H NMR (CDCl₃): δ 9.72 (bs, 1H), 9.12 & 8.83 (2 s, 1H, rotamers in a ratio of 4:1), 8.87 (s, 1H), 8.87 (s, 1H), 8.38-8.18 (m, 3H), 8.03 (s, 1H), 4.77 & 4.45 (2 d, 2H, J=5.4 Hz, rotamers in a ratio of 1:4), 3.97 & 3.91 (2 s, 3H); MS (m/e): 334 (M⁺+1), 305, 290, 215; IR (KBr, cm⁻¹): 3447, 3184, 2925, 1530, 1215, 771.

### EXAMPLE 21

### N-[1-(3,5-Difluoro-4pyrrol-1-yl-phenyl)-1H-[1,2,3]triazol-4-ylmethyl]-acetamide

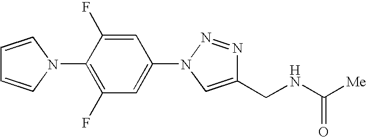

To a DMF solution (3 mL) of 1-(4-azido-2,6-difluorophenyl)-1H-pyrrole (350 mg, 1.59 mmol), obtained in preparation 38, was added propargyl amine N-acetamide (232 mg, 2.38 mmol) followed by the addition of N,N-diisopropylethylamine (205 mg, 1.59 mmol). Cuprous iodide (604 mg, 3.18 mmol) was added to the reaction mixture in portion and stirred at room temperature for 0.5 hours. A saturated solution of ammonium chloride (50 mL) was added followed by few drops of ammonium hydroxide and then the reaction mixture was extracted with ethylacetate (75 mL×2). Combined organic phase was washed with brine and dried over sodium sulfate. Removal of volatiles and purification of the resulting residue through silica gel column (pet. ether/ethylacetate, 1:1) yielded 100 mg (20%) of the title compound.

¹H NMR (CDCl₃): δ 8.05 (s, 1H), 7.52 (d, J=8.3 Hz, 2H), 6.95 (s, 2H), 6.41 (s, 2H), 6.31 (bs, 1H), 4.58 (d, J=5.6 Hz, 2 H), 2.03 (s, 3H); MS (m/e): 318 (M⁺+1), 289; IR (KBr, cm⁻¹): 3263, 2925, 1636, 1537.

### EXAMPLE 22

### N-[1-(3-Fluoro-4-pyrrol-1-yl-phenyl)-1H-[1,2,3]triazol-4-ylmethyl]-acetamide

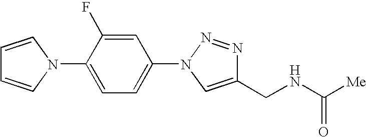

To an ice cooled DMF solution (3 mL) of 1-(4-azido-2-fluorophenyl)-1H-pyrrole (300 mg, 1.49 mmol), obtained in preparation 12, was added propargyl amine N-acetamide (173 mg, 1.78 mmol) followed by the addition of N,N-diisopropylethylamine (192 mg, 1.49 mmol). Cuprous iodide (566 mg, 2.98 mmol) was added to the reaction mixture in portion and stirred at the same temperature for 15 min., then at room temperature for 2 hours. A saturated solution of ammonium chloride, (50 mL) was added followed by few drops of ammonium hydroxide and then the reaction mixture was extracted with ethylacetate (75 mL×2). Combined organic phase was washed with brine and dried over sodium sulfate. Removal of volatiles and purification of the resulting residue through silica gel column (pet. ether/ethylacetate, 1:2) yielded 250 mg (56%) of the title compound. Mp. 210-215° C.

¹H NMR (CDCl₃): δ 8.03 (s, 1H), 7.74-7.54 (m, 3H), 7.09 (d, J=2.1 Hz, 2H), 6.40 (s, 2H), 6.25 (bs, 1H), 4.59 (d, J=5.9 Hz, 2H), 2.04 (s, 3H); MS (m/e): 300 (M⁺+1), 186; IR (KBr, cm⁻¹): 3303, 2925, 1659, 1536.

### EXAMPLE 23

### N-[1-(3-Fluoro-4-imidazol-1-yl-phenyl)-1H-[1,2,3]triazol-4-ylmethyl]-acetamide

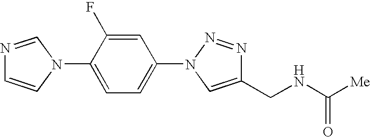

The title compound was prepared from 1-(4-azido-2-fluorophenyl)-1H-imidazole, obtained in preparation 28, and propargyl amine N-acetamide following the procedure reported in example 22. Mp. 198-199° C.

¹H NMR (CDCl₃+DMSO-d₆): δ 8.40 (s, 1H), 8.18 (bs, 1H), 8.01-7.50 (m, 4H), 7.41 (s, 1H), 7.20 (s, 1H), 4.50 (d, J=5.4 Hz, 2 H), 1.98 (s, 3H); MS (m/e): 301 (M⁺+1), 273; IR (KBr, cm⁻¹): 3230, 3045, 1666, 1533.

### EXAMPLE 24

### N-[1-(3-Fluoro-4-imidazol-1-yl-phenyl)-1H-[1,2,3]triazol-4-ylmethyl]-thioacetamide

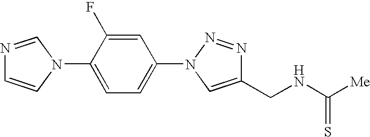

A mixture of Lawesson's reagent (94 mg, 0.23 mmol) and N-[1-(3-fluoro-4-imidazol-1-yl-phenyl)-1H-[1,2,3]triazol-4-ylmethyl]-acetamide (100 mg, 0.33 mmol), obtained in example 23, in dry dioxane (5 mL) was heated to 60° C. for 2 hours. The reaction mixture was then diluted with ethyl acetate (100 mL) and washed with water followed by brine and dried over sodium sulfate. Removal of volatiles and purification of the resulting residue by column chromatography (chloroform/methanol, 96:4) yielded the title compound as cream color powder (50 mg, 48%). Mp. 185° C.

¹H NMR (CDCl₃+DMSO-d₆): δ 10.35 (bs, 1H), 8.67 (s, 1H), 8.01-7.65 (m, 4H), 7.43 (s, 1H), 7.12 (s, 1H), 4.89 (d, J=4.9 Hz, 2 H), 2.48 (s, 3H); MS (m/e): 317 (M⁺+1), 289, 248, 214; IR (KBr, cm⁻¹): 3238, 2922, 1531.

### EXAMPLE 25

### N-[1-(3-Fluoro-4-[1,2,4]triazol-1-yl-phenyl)-1H-[1,2,3]triazol-4-ylmethyl]-acetamide

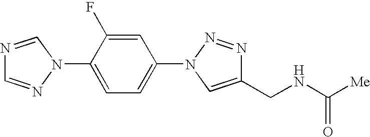

To a DMF solution (5 mL) of 1-(4-azido-2-fluorophenyl)-1H-[1,2,4]triazole, obtained in preparation 36, and N,N-diisopropylethyl amine (157 mg, 1.2 mmol) was added propargyl N-acetamide (142 mg, 1.46 mmol) followed by the addition of cuprous iodide (228 mg, 1.2 mmol) in portion and stirred at room temperature for 0.5 hours. A saturated solution of ammonium chloride (10 mL) was added to the reaction mixture followed by the addition of few drops of ammonium hydroxide. The reaction mixture was then diluted with ethyl acetate (100 mL) and the aqueous layer was separated. The organic phase was washed with water followed by brine and dried over sodium sulfate. Evaporation of volatiles and purification of the resulting residue through a silica gel column yielded the title compound as white powder (105 mg, 28%). Mp. 223° C.

¹H NMR (CDCl₃): δ 9.12 (s, 1H), 8.79 (s, 1H), 8.47 (bs, 1H), 8.24 (d, J=12.7 Hz, 1H), 8.03 (bs, 2H), 4.40 (d, J=5.3 Hz, 2 H), 1.88 (s, 3H); MS (m/e): 302 (M⁺+1), 273 181; IR (KBr, cm^{31 1}): 3286, 2925, 1670, 1259.

### EXAMPLE 26

### N-[1-(3-Fluoro-4-[1,2,4]triazol-1-yl-phenyl)-1H-[1,2,3]triazol-4-ylmethyl]-thioacetamide

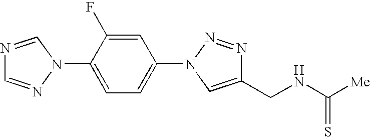

The title compound was prepared from N-[1-(3-fluoro-4-[1,2,4]triazol-1-yl-phenyl)-1H-[1,2,3]triazol-4-ylmethyl]acetamide, obtained in example 25, and Lawesson's reagent by following the similar procedure as described in example 24.

¹H NMR (CDCl₃+DMSO-d₆): δ 10.41 (bs, 1H), 8.91 (d, J=2.44 Hz, 1H), 8.77 (s, 1H), 8.21-7.95 (m, 3H), 7.43 (s, 1H), 4.92 (d, J=4.9 Hz, 2 H), 2.50 (s, 3H); MS (m/e): 318 (M⁺+1), 302, 289, 215; IR (KBr, cm⁻¹): 3234, 3214, 1532.

### EXAMPLE 27

### {1-[3-Fluoro-4-(4-hydroxymethyl-imidazol-1-yl)-phenyl]-1H-[1,2,3]triazol-4-ylmethyl}-thiocarbamic acid O-methyl ester

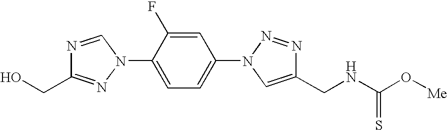

To a DMF solution (5 mL) of [1-(4-Azido-2-fluoro-phenyl)-1H-imidazol-4-yl]-methanol (500 mg, 2.14 mmol), obtained in preparation 42, and diisopropylethyl amine (332 mg, 12.57 mmol) was added prop-2-ylnyl-thiocarbamic acid O-methyl ester (332 mg, 2.57 mmol) followed by the addition of cuprous iodide (815 mg, 4.28 mmol) in portion and stirred at room temperature for 0.5 hours. Saturated solution of ammonium chloride (10 mL) was added to the reaction mixture followed by the addition of few drops of ammonium hydroxide solution. The blue colour solution was diluted with ethyl acetate (100 mL) and aqueous layer was separated. The organic layer was washed with water followed by brine and dried over sodium sulfate. Evaporation of volatiles on rotavapor and purification of the resulting residue through silica gel column yielded the white color compound (300 mg, 40%). Mp. 179° C.

¹H NMR (DMSO-d₆): δ 9.71 (bs, 1H), 8.83 & 8.79 (2s, 1H, rotamers in a ratio of 4:1), 8.18 (d, J=11.8 Hz, 1H), 8.00 (s, 1H), 7.89-7.80 (m, 2H), 7.46 (s, 1H), 5.04 (bs, 1H), 4.75 (d, J=5.6 Hz, 2H), 4.43 (d, J=5.4 Hz, 2H), 3.95 & 3.89 (2s, 3H, rotamers in a ratio of 1:4); MS (m/e): 363 (M⁺+1), 331; IR (KBr, cm⁻¹): 3427, 2925, 2854, 2689, 1536.

### EXAMPLE 28

### {1-[3,5-Difluoro-4-(4-hydroxymethyl-imidazol-1-yl)-phenyl]-1H-[1,2,3]triazol-4-ylmethyl}-thiocarbamic acid O-methyl ester

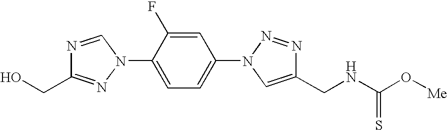

The title compound was prepared, by following a same procedure reported for the preparation of example 27, starting from the 3,4,5-trifluoronitrobenzene.

¹H NMR (CDCl₃+DMSO-d₆): δ 9.73 (bs, 1H), 8.88 & 8.83 (2s, 1H, rotamers in a ratio of 4:1), 8.11 (d, J=8.8 Hz, 1H), 7.93 (s, 1H), 7.34 (s, 1H), 5.05 (t, J=5.6 Hz, 1H), 4.77 (d, J=5.4 Hz, 2H), 4.30 (d, J=5.6 Hz, 2H), 3.89 & 3.33 (2s, 3H, rotamers in a ratio of 1:4). MS (m/e): 381(M⁺+1), 349, 192; IR (KBr, cm⁻¹): 3446, 1537.

### EXAMPLE 29

### {1-[4-(4-Cyano-imidazol-1-yl)-3-fluoro-phenyl]-1H-[1,2,3]triazol-4-ylmethyl}-thiocarbamic acid O-methyl ester

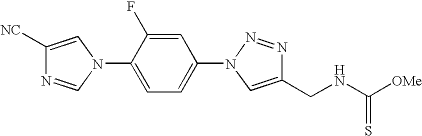

The title compound was prepared from 1-(4-azido-2-fluoro-phenyl)-1H-imidazole-4-carbonitrile (230 mg, 1.0 mmol) following the procedure described in example 10 (yield, 150 mg, 40%).

IR (KBr, cm⁻¹): 3327, 3127, 2925, 2238, 1538.

¹H NMR (DMSO-d₆): δ 9.71 (bs, 1H), 8.86 & 8.82 (2s, in a ratio of 4:1, 1H), 8.67 (s, 1H), 8.39 (s, 1H), 8.25 (d, J=11.5 Hz, 1H), 7.90-8.10 (m, 2H), 4.76 & 4.50 7.43 (2d, rotamers in a ratio of 4:1, J=5.6 Hz, 1H), 3.95 & 3.89 (2s, rotamers in a ratio of 1:4, 3H).

MS (m/e): 358 (M⁺+1), 326, 269.

### EXAMPLE 30

### {1-[4-(4-Cyano-imidazol-1-yl)-3,5-difluoro-phenyl]-1H-[1,2,3]triazol-4ylmethyl}-thiocarbamic acid O-methyl ester

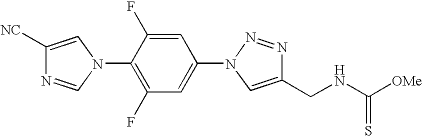

This compound was prepared by following the procedure as described in Example 10.

IR (KBr, cm−1): 3369, 2242, 1531.

¹H NMR (DMSO-d₆): δ 9.74 (bs, 1H), 8.90 & 8.85 (2s, rotamers in a ratio of 4:1, 1H), 8.62 (s, 1H), 8.35 (s, 1H), 8.19 (d, J=8.9 Hz, 2H), 4.76 & 4.47 (2d, rotamers in a ratio 6f 4:1, J=5.6 Hz, 2H), 3.95 & 3.89 (2s, rotamers in a ratio of 1:4, 3H).

MS (m/e): 376 (M⁺+1), 344, 257.

### In Vitro Data

Minimum Inhibiton Concentrations (MICs) were determined by broth microdilution technique as per the guidelines prescribed in the fifth edition of Approved Standards, NCCLS document M7-A5 Vol 20-No 2, 2000 Villinova, Pa.

Initial stock solution of the test compound was prepared in DMSO. Subsequent two fold dilutions were carried out in sterile Mueller Hinton Broth (Difco) (MHB).

Frozen cultures stocks were inoculated into 50 ml sterile MHB in 250 ml Erlyn Meyer flasks.
Composition of MHB is as follows:
Beef Extract Powder—2.0 grams/litre
Acid Digest of Casein—17.5 grams/litre
Soluble Starch—1.5 grams/litre
Final pH 7.3±0.1

Flasks were incubated for 4 to 5 hours at 35° C. on a rotary shaker at 150 rpm. Inoculum was prepared by diluting the culture in sterile MHB to obtain a turbidity of 0.5 McFarland standard. This corresponds to 1-2×10⁸ CFU/ml. The stock was further diluted in sterile broth to obtain 1-2×10⁶ CFU/ml. 50 μl of the above diluted inoculum was added from 1-10 wells. The plates were incubated overnight at 37° C.

MIC is read as the lowest concentration of the compound that completely inhibits growth of the organism in the microdilution wells as detected by the unaided eye.

| Organism | Culture No. | DRCC No. |
|---|---|---|
| Staphylococcus aureus | ATCC 33591 | 019 |
| Staphylococcus aureus | ATCC 49951 | 213 |
| Staphylococcus aureus | ATCC 29213 | 035 |
| Enterococcus faecalis | ATCC 29212 | 034 |
| Enterococcus faecalis | NCTC 12201 | 153 |
| Enterococcus faecium | NCTC 12202 | 154 |
| Escherichia coli | ATCC 25922 | 018 |
| Haemophilus influenzae | ATCC 49247 | 432 |
| Haemophilus influenzae | ATCC 49766 | 433 |
| Haemophilus influenzae | ATCC 9006 | 529 |
| Moraxella catarrhalis | ATCC 25238 | 300 |
| Streptococcus pneumoniae | ATCC 6303 | 236 |
| Streptococcus pneumoniae | ATCC 49619 | 237 |
| Streptococcus pneumoniae | ATCC 700673 | 238 |
| S. aureus - MRSA | — | 446 |
| S. aureus - MRSA | — | 448 |
| S. aureus - MRSA | — | 449 |
| Corynebacterium jeikeium | | |
| Viridans Streptococci | | |

ATCC: American Type Culture Collection, USA
NCTC: National Collections of Type Cultures, Colindale, UK
DRCC: Dr. Reddy's Culture Collection, Hyderabad, India.

The in vitro antibacterial activity data is shown in TABLE 1.

TABLE 1
In vitro Activity of Compounds against Gram
positive and Gram negative bacteria

| | Antimicrobial Screening (MIC) μg/mL | | | | | | | |
|---|---|---|---|---|---|---|---|---|
| | Staphylococcus | | | | | | | |
| Ex- | aureus | | | | | | | |
| am- | | 213 | | Enterococcus sp | | | Myco- | Salmo- |
| ple | 019 | Smith | 035 | 034 | 153 | 154 | bacterium | nella |
| No. | MRSA | S | S | S | R | R | MTCC006 | TA97 |
| 8 | 0.5 | 0.5 | 1 | 0.5 | 0.5 | 0.5 | 32 | 32 |
| 9 | 1 | 1 | 1 | 2 | 1 | 2 | 4 | 32 |
| 20 | 8 | 16 | 16 | 32 | 32 | 32 | 16 | 32 |
| 27 | 1 | 1 | 1 | 2 | 1 | 2 | 16 | 32 |

We claim:

1. A compound of formula (I) 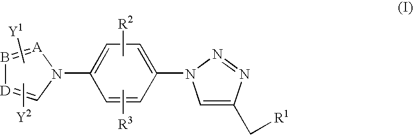
where R¹ represents halogen, azido, thioalcohol, isothiocyanate, hydroxy, isoindole-1,3-dione, substituted or unsubstituted (C1-C20)alkylsulfonyloxy, arylsulfonyloxy, (C1-C20)acyloxy group, NHR⁴ where R⁴ represents hydrogen, substituted or unsubstituted groups selected from (C1-C20)alkyl, (C1-C20)acyl, thio(C1-C20)acyl, (C1-C20)alkoxycarbonyl, (C3-C20)cycloalkoxycarbonyl, (C3-C20)cycloalkoxythiocarbonyl, (C2-C20)alkenyloxycarbonyl, (C2-C20)alkenylcarbonyl, heteroaryl, aryloxycarbonyl, heteroarylcarbonyl, heteroarylthiocarbonyl, (C1-C20)alkoxythiocarbonyl, (C2-C20)alkenyloxythiocarbonyl, aryloxythiocarbonyl, —C(═O)—C(═O)—(C1-C20)alkyl, —C(═O)—C(═O)-aryl, —C((═O)—C(═O)—(C1-C20)alkoxy, —C(═O)—C(═O)-aryloxy, —C(═O)—C(═S)—(C1-C20)alkyl, —C(═O)—C(═S)-aryl, —C(═S)—S—(C1-C20)alkyl, —C(═S)—NH₂, —C(═S)—NH—(C1-C20)alkyl, —C(═S)—N—((C1-C20)alkyl)₂, —C(═S)—NH—(C2-C20)alkenyl, —C(═S)—C(═O)—(C1-C20)alkoxy, —C(═S)—C(═O)-aryloxy, —C(═S)—O—C(═O)—(C1-C20)alkyl, —C(═S)—C(═S)—(C1-C20)alkyl, —C(═S)—C(═S)-aryl, —C(═S)—NH—C(═O)-aryl, —C(═S)—NH-aralkyl, —C(═S)—NH-heteroaralkyl, —C(═NH)—NH₂, —C(═NH)—(C1-C20)alkyl, —C(═NH)-aryl, —S(O)₂(C1-C20)alkyl, —S(O)₂aryl, thiomorpholinylthiocarbonyl, pyrrolidinylthiocarbonyl or —C(═S)—N(R′R″), where R′ and R″ together form a substituted or unsubstituted 5 or 6 member heterocycle ring containing nitrogen and optionally having one or two additional hetero atoms selected from O or S; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkyl carbonyl, (C1-C20)alkoxycarbonyl, carboxy(C1-C20)alkyl, (C1-C20)alkylsulfonyl, (C1-C20)alkyl carbonylamino(C1-C20)alkyl, arylcarbonylamino(C1-C20)alkyl, (C1-C20)alkyl aminocarbonyl, (C1-C20)alkylcarbonyloxy(C1-C20)alkyl, amino(C1-C20)alkyl, mono(C1-C20)alkyl amino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH; their pharmaceutically acceptable salts.

2. The compound of formula (I), as claimed in claim 1, wherein a substituted or unsubstituted 5 or 6 member heterocycle ring formed by R′ & R″, containing nitrogen, optionally having one or two additional heteroatoms selected from oxygen, nitrogen or sulfur, is selected from pyrrolidinyl, pyrrolyl, morpholinyl, thiomorpholinyl, benzothiazole, benzoimidazolyl, pyridinyl, pyridazinyl, pyrimidinyl or pyrazinyl.

3. The compound of formula (I), as claimed in claim 1, wherein the substituents on R⁴, 4^{4a}, 4^{4b}, 4^{4c}, 4^{4d}, 4^{4e}, R, R⁷ and the heterocycle formed by R′ and R″ are selected from halogen atom, hydroxy, amino, cyano, nitro, (C1-C20)alkyl, hydroxy(C1-C20)alkyl, (C1-C20)alkoxy, ═O, ═S, aryl, hydroxyaryl, pyridyl, mono(C1-C20)alkylamino, di(C1-C20)alkylamino, (C1-C20)acyl, thio(C1-C20)acyl, (C1-C20)alkoxycarbonyl, (C1-C20)alkoxyaryl or carboxylic acid or its derivatives.

4. The compound of formula (I), as claimed in claim 1, wherein the substituents on R² and R³ are selected from the group consisting of hydroxy, halogen, nitro, amino, (C1-C20)alkyl, (C1-C20)alkoxy, ═O, ═S, cyano group or carboxylic acid or its derivatives.

5. The compound of formula (I), as claimed in claim 1, wherein the substituents on R^{a} are selected from from hydroxy, halogen, nitro, amino, (C1-C20)alkyl, (C1-C20)alkoxy, cyano group, or carboxylic acid or its derivatives.

6. The compound of formula (I), as claimed in claim 1, wherein the substituents on Y¹ and Y² are selected from hydroxy, nitro, cyano, amino, tert-butyldimethylsilyloxy (TBSO), halogen atom, (C1-C20)alkyl, (C1-C20)alkoxy, (C3-C20)cycloalkyl, aryl, benzyloxy, acyl or acyloxy group.

7. The compound of formula (I), as claimed in claim 1, wherein the substituents on R^{c} and R^{d} are selected from halogen, hydroxy, nitro, amino, cyano, (C1-C20)alkyl or (C1-C20) alkoxy.

8. The compound of formula (I), as claimed in, wherein R¹ represents NHR⁴ where R⁴ represents (C1-C20)acyl, C1-C20)alkoxycarbonyl;
R² and R³ may be same or different and independently represent hydrogen, halogen atom, (C1-C20)alkyl group, halo(C1-C20)alkyl;
Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkyl carbonyl, (C1-C20)alkoxycarbonyl, carboxy(C1-C20)alkyl, (C1-C20)alkylsulfonyl, (C1-C20)alkylcarbonylamino(C1-C20)alkyl, (C1-C20)alkylaminocarbonyl, arylcarbonylamino(C1-C20)alkyl, (C1-C20)alkylcarbonyloxy(C1-C20)alkyl, amino(C1-C20)allyl, mono(C1-C20)alkylamino, di(C1-C20)alkylamino, (C1-C20)alkoxy, or any one or two of Y¹ or Y² may represent substituted or unsubstituted —CH═NOR′″, wherein R′″ represents hydrogen and (C1-C20)alkyl group, carboxylic acid or its derivatives.

9. The compound of formula (I), as claimed in claim 1, wherein R¹ represents NHR⁴ where R⁴ represents (C1-C20)acyl, C1-C20)alkoxycarbonyl;
R² and R³ may be same or different and independently represent hydrogen, halogen atom, halo(C1-C20)alkyl;
Y¹ and Y² may be same or different and independently represent hydrogen, cyano, halogen, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkylcarbonylamino(C1-C20)alkyl, (C1-C20)alkylaminocarbonyl, amino(C1-C20)alkyl, mono(C1-C20)alkylamino, di(C1-C20)alkylamino, (C1-C20)alkoxy, or any one or two of Y¹ or Y² may represent substituted or unsubstituted —CH═NOH, carboxylic acid or its derivatives. The substituents on Y¹ and Y² may be selected from hydroxy, cyano, amino, (C1-C20)alkyl, (C1-C20)alkoxy, acyl, COOR^{c}, wherein R^{c} represents hydrogen or (C1-C20)alkyl.

10. The compound of formula (I), as claimed in claim 1, wherein R¹ represents NHR⁴ where R⁴ represents thio(C1-C20)acyl, (C1-C20)alkoxythiocarbonyl,
R² and R³ may be same or different and independently represent hydrogen, halogen atom, (C1-C20)alkyl group, halo(C1-C20)alkyl;
Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, (C1-C20)alkylaminocarbonyl, arylcarbonylamino(C1-C20)alkyl, (C1-C20)alkylcarbonyloxy(C1-C20)alkyl, amino(C1-C20)alkyl, mono(C1-C20)alkylamino, di(C1-C20)alkylamino, (C1-C20)alkoxy, or any one or two of Y¹ or Y² may represent substituted or unsubstituted —CH═NOR′″, wherein R′″ represents hydrogen and (C1-C20)alkyl group, carboxylic acid or its derivatives.

11. The compound of formula (I), as claimed in claim 1, wherein R¹ represents NHR⁴ where R⁴ represents thio(C1-C20)acyl, (C1-C20)alkoxythiocarbonyl,
R² and R³ may be same or different and independently represent hydrogen, halogen atom, halo(C1-C20)alkyl;
Y¹ and Y² may be same or different and independently represent hydrogen, cyano, halogen, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkylcarbonylamino(C1-C20)alkyl, (C1-C20)alkylaminocarbonyl, amino(C1-C20)alkyl, mono(C1-C20)alkylamino, di(C1-C20) alkylamino, (C1-C20)alkoxy, or any one or two of Y¹ or Y² may represent substituted or unsubstituted —CH═NOH, carboxylic acid or its derivatives. The substituents on Y¹ and Y² may be selected from hydroxy, cyano, amino, (C1-C20)alkyl, (C1-C20)alkoxy, acyl, COOR^{c}, wherein R^{c} represents hydrogen or (C1-C20)alkyl.

12. The compound of formula (I), as claimed in claim 1, which is

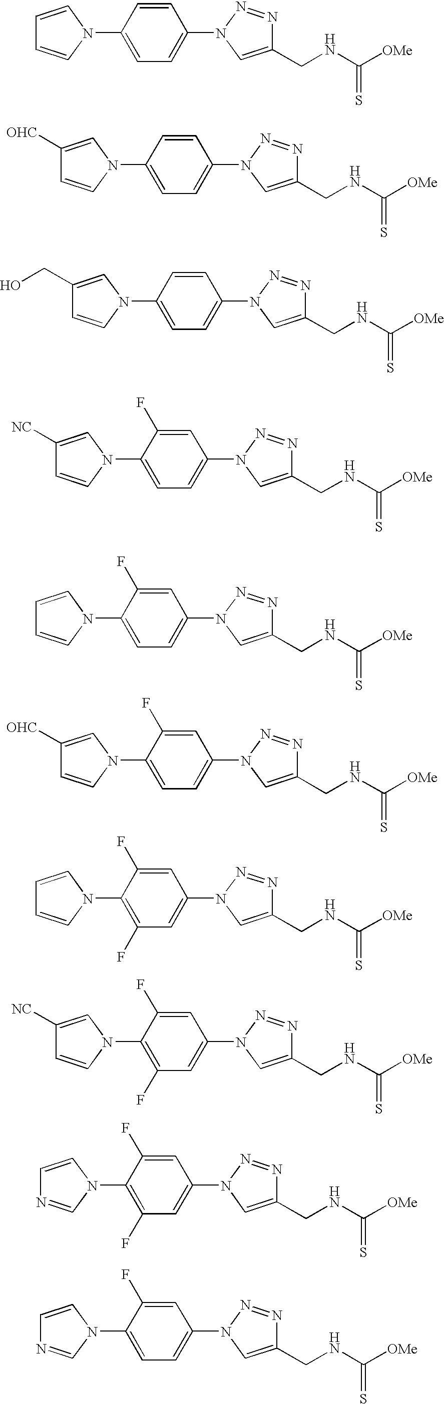

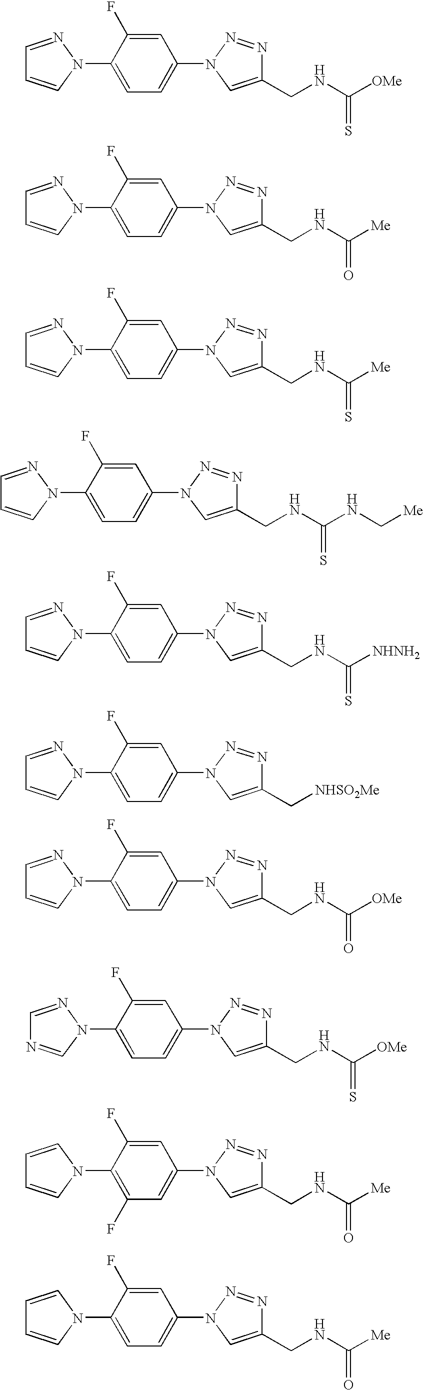

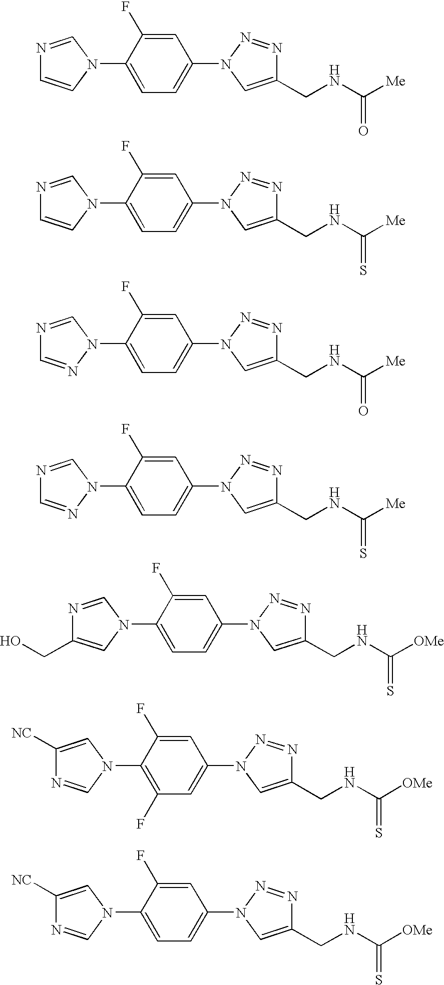

13. The compound of formula (I), as claimed in claim 1, which is

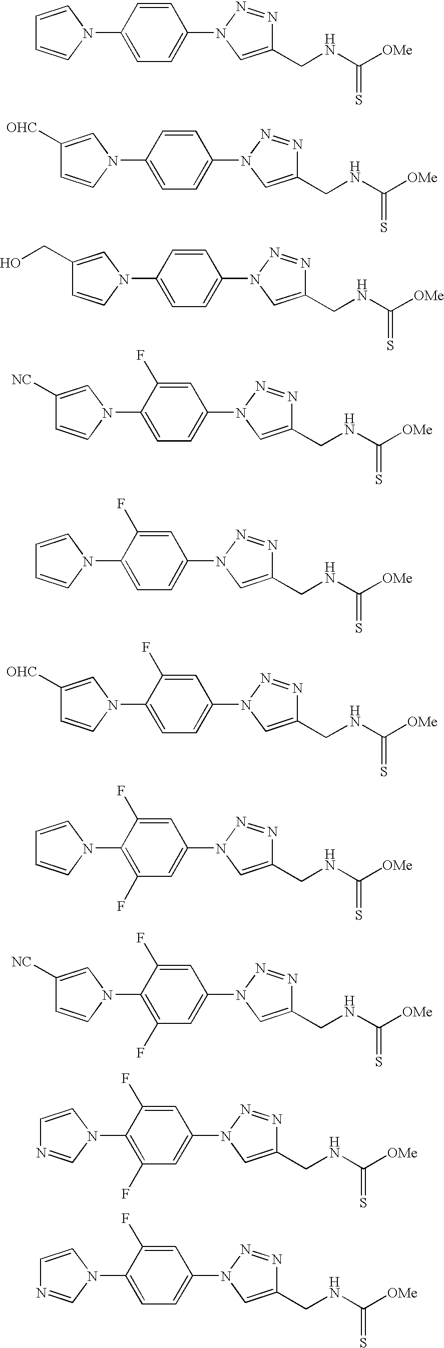

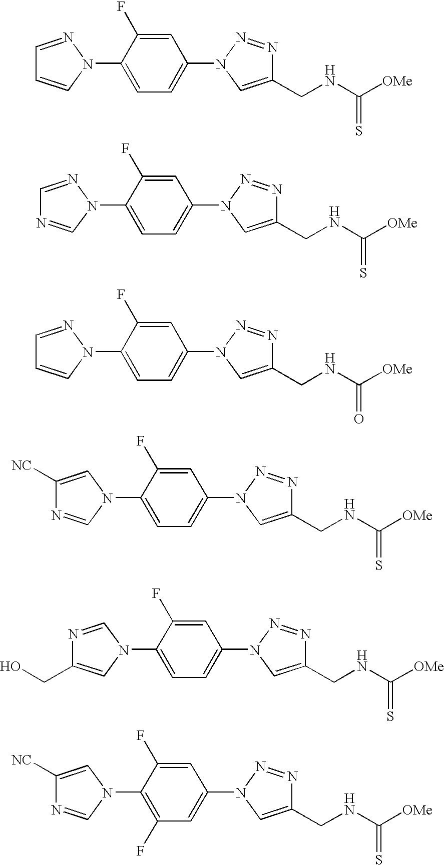

14. The compound of formula (I), as claimed in claim 1, which is

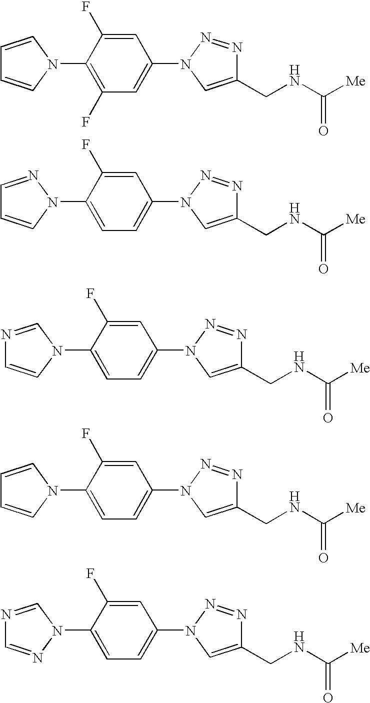

15. The compound of formula (I), as claimed in claim 1, which is

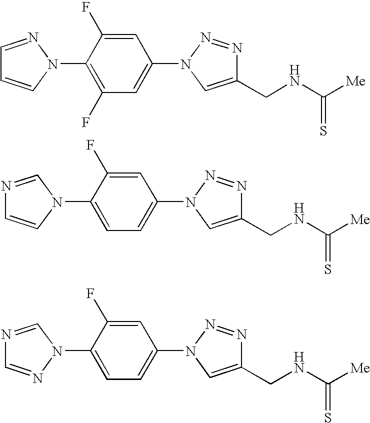

16. The compound of formula (I), as claimed in claim 1, which is

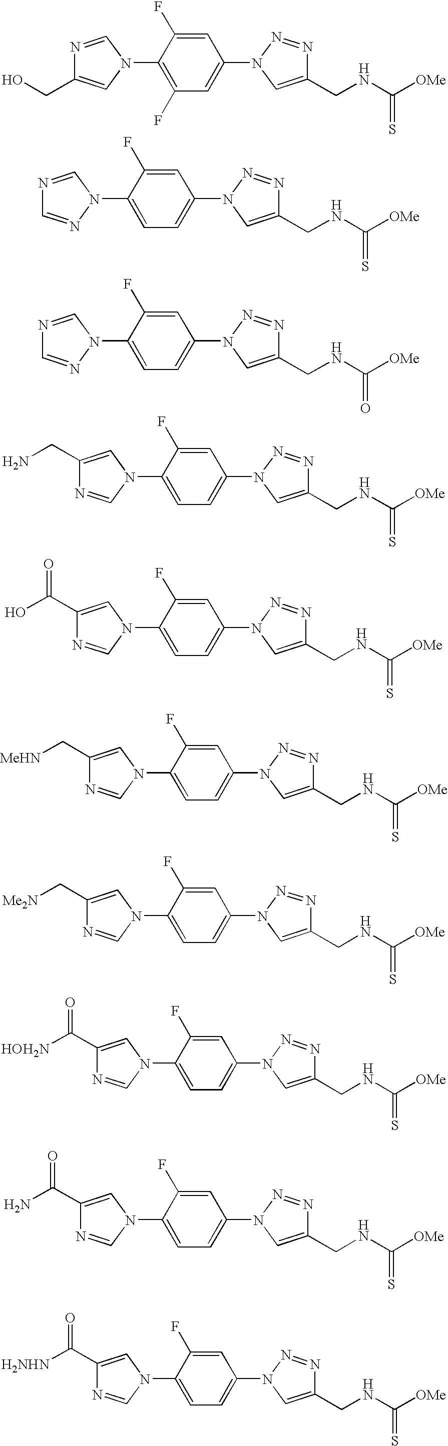

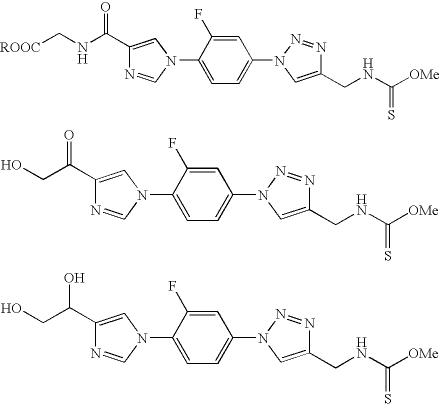

17. The compound of formula (I), as claimed in claim 1 is

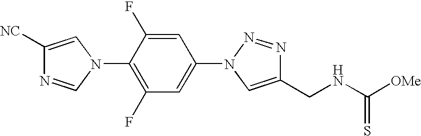

18. The compound of formula (I), as claimed in claim 1 is

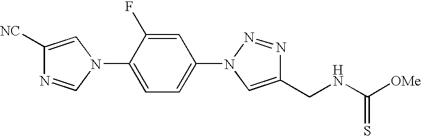

19. The compound of formula (I), as claimed in claim 1 is

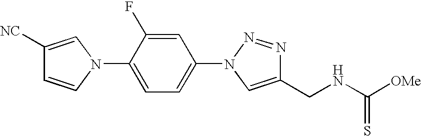

20. The compound of formula (I), as claimed in claim 1 is

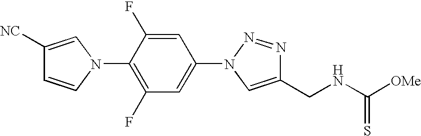

21. The compound of formula (I), as claimed in claim 1 is

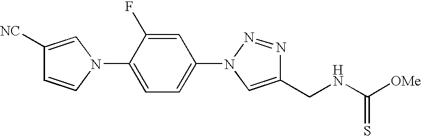

22. A preferred compound of the present invention is

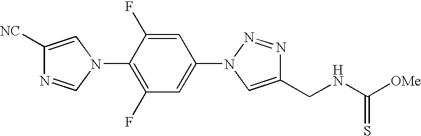

23. A process for the preparation of the compound of formula (I) 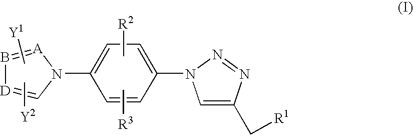
where R¹ represents azido group; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, substituted or unsubstituted groups selected from (C1-C20)alkyl, hydroxy(C1-C20)allyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises:
(a) (i) reacting the compound of formula (Ia) 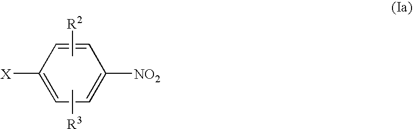
where X represents halogen atom; R² and R³ are as defined above, with a compound of formula (Ib) 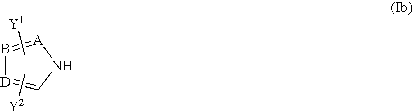
where A, B, D, Y¹ and Y² are as defined above, to produce a compound of formula (Ic) 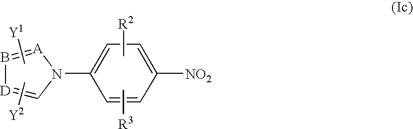
where A, B, D, Y¹, Y², R² and R³ are as defined above,
(ii) reducing the compund of formula (Ic) by using reducing agent, to a compound of formula (Id) 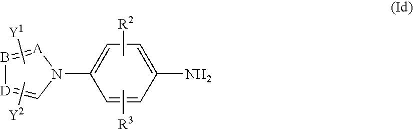
where A, B, D, Y¹, Y², R² and R³ are as defined above,
(iii) converting the compound of formula (Id) to a compound of formula (Ie) 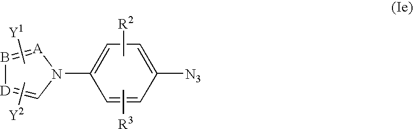
where A, B, D, Y¹, Y², R² and R³ are as defined above,
(iv) converting the compound of formula (Ie) to a compound of formula (If) 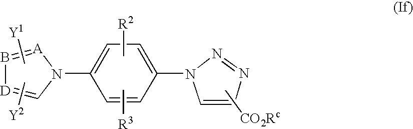
where R^{c} represents substituted or unsubstituted (C1-C20)alkyl group; A, B, D, Y¹, Y², R² and R³ are as defined above,
(v) reducing the compound of formula (If), to give a compound of formula (I) 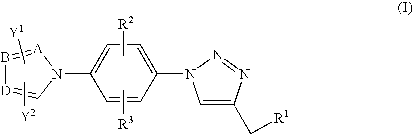
where R¹ represents hydroxy group; A, B, D, Y¹, Y², R² and R³ are as defined above,
(vi) converting the compound of formula (I), where R¹ represents hydroxy group, to a compound of formula (I), where R¹ represents substituted or unsubstituted (C1-C20)alkylsulfonyloxy or arylsulfonyloxy group and all other symbols are as defined above, and
(vii) converting the compound of formula (I) where R¹ represents substituted or unsubstituted (C1-C20)alkylsulfonyloxy or arylsulfonyloxy group, to a compound of formula (I) where R¹ represents azido group and all other symbols are as defined above or
(b) (i) reacting the compound of formula (Ia) 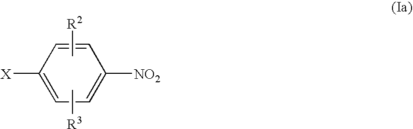
where X represents halogen atom; R² and R³ are as defined earlier, with a compound of formula (II) 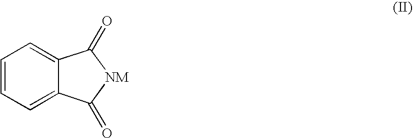
where M represents metal atom such as sodium, potassium and the like, to produce a compound of formula (2m) 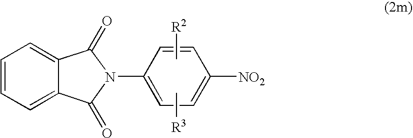
where R² and R³ are as defined above,
(ii) reducing the compound of formula (Im) by using reducing agent to a compound of formula (In) 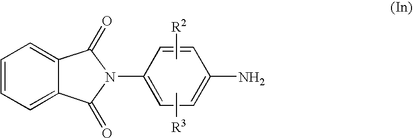
where R² and R³ are as defined above,
(iii) converting the compound of formula (In) to a compound of formula (Io) 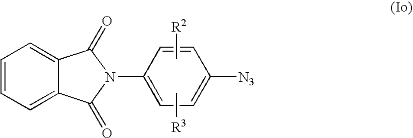
where R² and R³ are as defined above,
(iv) reacting the compound of formula (Io), with compound of formula (Ip) 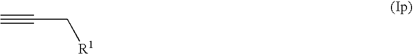
where R¹ is as defined above, to obtain a compound of formula (Iq) 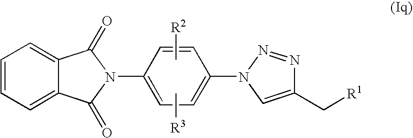
where R¹ represents NHR⁴ wherein R⁴ is as defined above, R² and R³ are as defined above,
(v) converting the compound of formula (Iq), to a compound of formula (Ir) 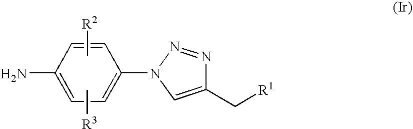
where R¹ is as defined above, R² and R³ are as defined above,
(vi) converting the compound of formula (Ir), to a compound of formula (I) 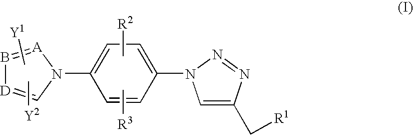
where R¹ is as defined above; R² and R³ are as defined above,
(c) (i) converting the compound of formula (Io) 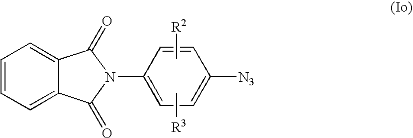
where R² and R³ are as defined above, to a compound of formula (Is) 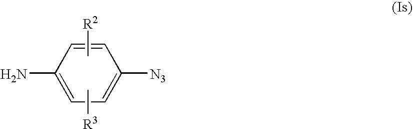
where R² and R³ are as defined above,
(ii) converting the compound of formula (Is), to a compound of formula (Ie) 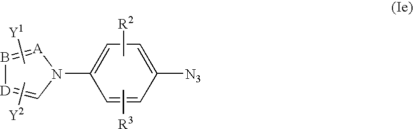
where all symbols are as defined above and
(iii) reacting the compound of formula (Ie), with a compound of formula (Ip) 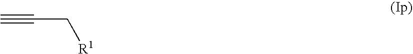
where R¹ is as defined in the description, to a compound of formula (I) 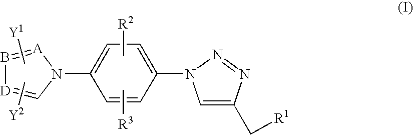
where R¹ is as defined above and all other symbols are as defined above.
(d) (i) converting the compound of formula (Ie) 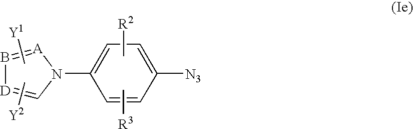
where A, B, D, Y¹, Y², R² and R³ are as defined above, to a compound of formula (I) 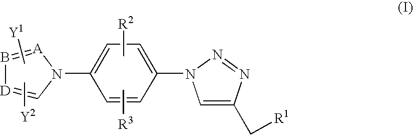
where R¹ represents hydroxy; A, B, D, Y¹, Y², R² and R³ are as defined above and
(ii) reacting the compound of formula (I) where R¹ represents hydroxy group, with MsCl, triethylamine and sodium azide to give a compound of formula (I) where R¹ represents azido group and all other symbols are as defined above or
(e) (i) converting the compound of formula (Ie) 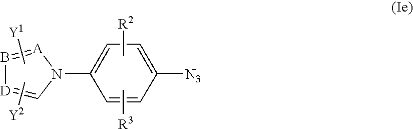
where A, B, D, Y¹, Y², R² and R³ are as defined above, to a compound of formula (I) 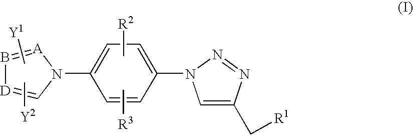
where R¹ represents halogen atom and all other symbols are as defined above and
(ii) converting the compound of formula (I) where R¹ represents halogen atom, to a compound of formula (I), wherein R¹ represents azido group.

24. A process for the preparation of compound of formula (I) 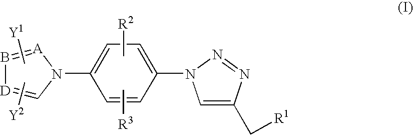
R¹ represents NHR⁴ wherein R⁴ represents hydrogen atom; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises:
(a) (i) converting the compound of formula (If) 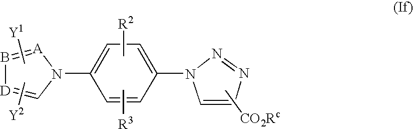
where R^{c} represents substituted or unsubstituted (C1-C20)alkyl group; A, B, D, Y¹, Y², R² and R³ are as defined above, to a compound of formula (Ig) 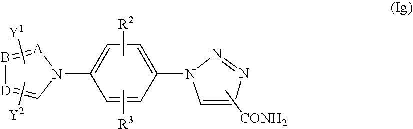
where all symbols are as defined above and
(ii) reducing the compound of formula (Ig), to produce a compound of formula (I) 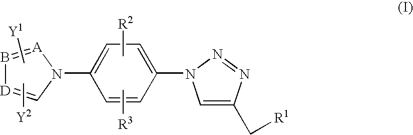
where R¹ represents NHR⁴ wherein R⁴ represents hydrogen atom and all other symbols are as defined above or
(b) (i) reducing the compound of formula (I) wherein R¹ represents azido group, to produce compound of formula (I) 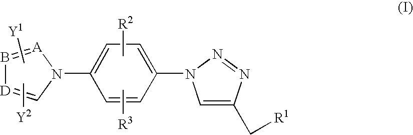
where R represents NHR⁴ wherein R⁴ represents hydrogen atom; A, B, D, Y¹, Y², R² and R³ are as defined above.

25. A process for the preparation of compound of formula (I) 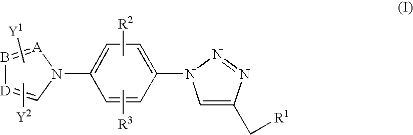
R¹ represents hydroxy group; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkylcarbonyloxy(C1-C20)alkyl, (C1-C20)alkylaminocarbonyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises:
(a) (i) reacting the compound of formula (Ia) 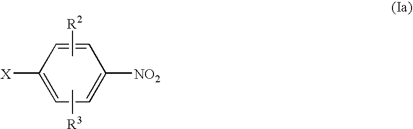
where X represents halogen atom;. R² and R³ are as defined above, with a compound of formula (Ib) 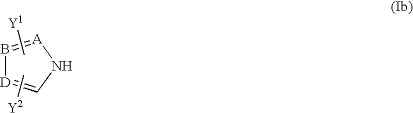
where A, B, D, Y¹ and Y² are as defined above, to produce a compound of formula (Ic) 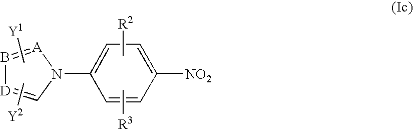
where A, B, D, Y¹, Y², R² and R³ are as defined above,
(ii) reducing the compund of formula (Ic) by using reducing agent, to a compound of formula (Id) 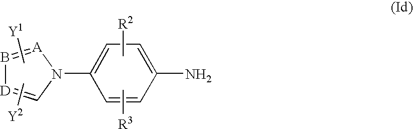
where A, B, D, Y¹, Y², R² and R³ are as defined above,
(iii) converting the compound of formula (Id) to a compound of formula (Ie) 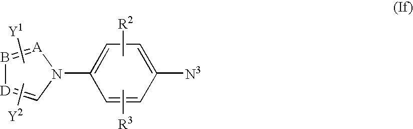
where A, B, D, Y¹, Y², R² and R³ are as defined above,
(iv) converting the compound of formula (Ie) to a compound of formula (If) 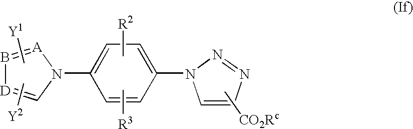
where R^{c} represents substituted or unsubstituted (C1-C20)alkyl group; A, B, D, Y¹, Y², R² and R³ are as defined above,
(v) reducing the compound of formula (If), to give a compound of formula (I) 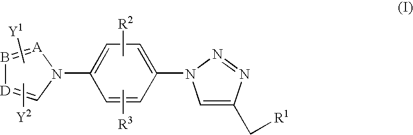
where R¹ represents hydroxy group; A, B, D, Y¹, Y², R² and R³ are as defined above, or
(b) (i) converting the compound of formula (Ie), 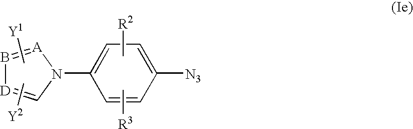
where A, B, D, Y¹, Y², R² and R³ are as defined above, to a compound of formula (I), 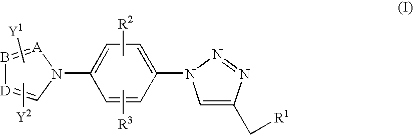
where R¹ represents substituted or unsubstituted (C1-C20)acyloxy group, and all other symbols are as defined above and
(ii) hydrolysis of the compound of formula (I) where R¹ represents (C1-C20)acyloxy group, to a compound of formula (I), where R¹ represents hydroxy group and all other symbols are as defined above.

26. A process for the preparation of compound of formula (I) 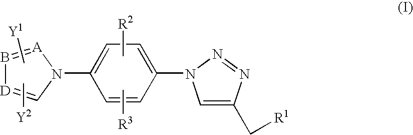
where R¹ represents NHR⁴, wherein R⁴ represents substituted or unsubstituted acetyl group; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises: reacting the compound of formula (I) where R¹ represents azido group and all other symbols are as defined above, with thiolacetic acid.

27. A process for the preparation of compound of formula (I) 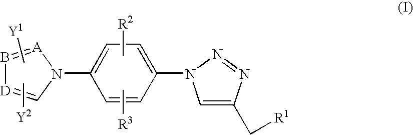
where R¹ represents NHR⁴, where R⁴ represents substituted or unsubstituted —C(═S)—R^{4a}, wherein R^{4a} represents (C1-C20)alkyl, halo(C1-C20)alkyl, aryl, heteroaryl, —C(═O)—(C1-C20)alkoxy, —C(═O)—(C1-C20)alkoxy, —C(═O)-aryloxy, —C(—S)—(C1-C20)alkyl or —C(═S)-aryl; R² and R³ may be same or different and independently represent hydrogen, halogen atom, substituted or unsubstituted (C1-C20)alkyl group, halo(C1-C20)allyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises: reacting the compound of formula (I) where R¹ represents NHR⁴, where R⁴ represents substituted or unsubstituted —C(═O)—R^{4a}, wherein R^{4a} represents (C1-C20)alkyl, halo(C1-C20)alkyl, aryl, heteroaryl, —C(═O)—(C1-C20)alkoxy, —C(═O)-aryloxy, —C(—S)—(C1-C20)alkyl or —C(═S)-aryl and all other symbols are as defined above, with a solution of amide and Lawesson's reagent (2,4-bis(methoxyphenyl)-1,3-dithia-2,4-diphosphetane-2,4-disulfide).

28. A process for the preparation of compound of formula (I) 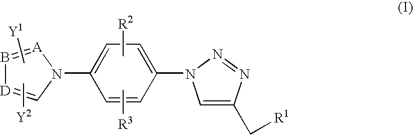
where R¹ represents NHR⁴, wherein R⁴ represents substituted or unsubstituted —C(═S)—OR^{4b}, wherein R^{4b} represents (C1-C20)alkyl, cyclo(C3-C20)alkyl, aryl, (C2-C20)alkenyl or —(C═O)—(C1-C20)alkyl group; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises:
(i) converting the compound of formula (I) 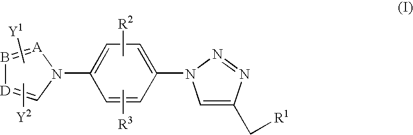
where R¹ represents azido group; and all other symbols are as defined above, to a compound of formula (1), where R¹ represents NHR⁴, wherein R⁴ represents hydrogen atom and all other symbols are as defined above,
(ii) converting the compound of formula (I, where R¹ represents NHR⁴, wherein R⁴ represents hydrogen atom, to a compound of formula (I), where R¹ represents isothiocyanate group; and all symbols are as defined above,
(iii) converting compound of formula (I) where R¹ represents isothiocynate group, to a compound of formula (I), where R¹ represents NHR⁴, wherein R⁴ represents substituted or unsubstituted —C(═S)—OR^{4b}, wherein R^{4b} is as defined above and all other symbols are as defined above.

29. A process for the preparation of compound of formula (I) 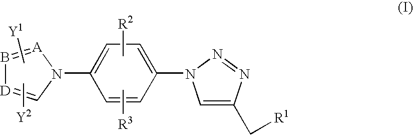
where R¹ represents NHR⁴, where R⁴ represents substituted or unsubstituted groups selected from —C(═S)—NH₂, —C(═S)—NH—(C1-C20)alkyl, —C(═S)—N—((C1-C20)alkyl)₂, —C(═S)—NH—(C2-C20)alkenyl, C(═S)—NH—C(═O)-aryl, —C(═S)—NH-aralkyl, —C(═S)—NH-heteroaralkyl or —C(═S)—N(R′R″), wherein R′ and R″ groups together form a substituted or unsubstituted 5 or 6 membered cyclic ring containing nitrogen and optionally having one or two additional hetero atoms selected from O or S; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy; amino, carboxyl or substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkyl aminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises: reacting a compound of formula (I) where R¹ represents isothiocyanate group and all other symbols are as defined above, with ammonia gas or amine.

30. A process for the preparation of compound of formula (I) 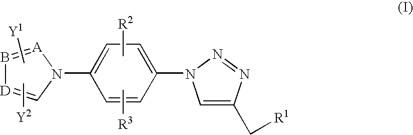
where R¹ represents NHR⁴, wherein R⁴ represents substituted or unsubstituted —C(═S)—SR^{4c}, wherein R^{4c} represents (C1-C20)alkyl group; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, carboxyl or substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises: reacting the compound of formula (I), where R¹ represents NHR⁴, wherein R⁴ represents hydrogen atom and all other symbols are as defined above, with CS₂ and alkylhalide.

31. A process for the preparation of compound of formula (I) 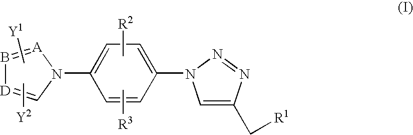
where R¹ represents NHR⁴, wherein R⁴ represents substituted or unsubstituted —C(═S)—NH—R^{4d}, wherein R^{4d} represents C(═O)-aryl group; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, carboxyl or substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkyl carbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkyl amino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises: reacting the compound of formula (I), where R¹ represents NHR⁴, wherein R⁴ represents hydrogen atom and all other symbols are as defined above, with benzoylisothiocyanate.

32. A process for the preparation of compound of formula (I) 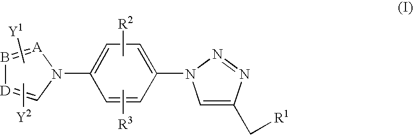
where R¹ represents NHR⁴, wherein R⁴ represents substituted or unsubstituted group selected from —C(═O)-heteroaryl; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, carboxyl or substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20) (C1-C20)alkylaminocarbonyl, alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH; their pharmaceutically acceptable salts and pharmaceutical compositions containing them, which comprises: reacting the compound of formula (I), where R¹ represents NHR⁴, wherein R⁴ represents hydrogen atom and all other symbols are as defined above, with heteroaryl acid chloride.

33. A process for the preparation of compound of formula (I) 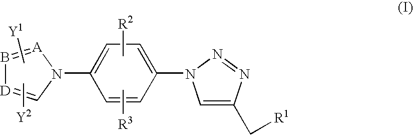
where R¹ represents NHR⁴ where R⁴ represents substituted or unsubstituted —(C═O)—R^{4e} wherein R^{4e} represents (C1-C20)alkyl, (C1-C20)alkoxy, (C2-C20)alkenyl, halo(C1-C20)alkyl, aryl, aryloxy, heteroaryl, (C2-C20)alkenyloxy, (C1-C20)alkylcarbonyl, arylcarbonyl, aryloxycarbonyl, (C1-C20)alkoxycarbonyl, (C1-C20)alkylthiocarbonyl or (C1-C20)arylthiocarbonyl; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, carboxyl or substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH; their pharmaceutically acceptable salts and pharmaceutical compositions containing them, which comprises: reacting the compound of formula (I), where R¹ represents NHR⁴, wherein R⁴ represents hydrogen atom and all other symbols are as defined above, with acid halide, alkylchloroformate or anhydride of acid.

34. A process for the preparation of compound of formula (I) 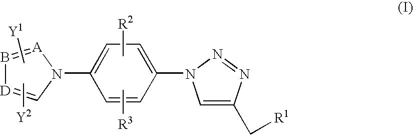
where R¹ represents NHR⁴ where R⁴ represents substituted or unsubstituted —C(═NH)—NH₂; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, carboxyl or substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkyl carbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)allyl, (C1-C20)alkoxy, aryl, heteroaryl and aralkyl group, carboxylic acid or its derivatives; A, B and D independently represent N or —CH, which comprises:
(a) reacting a compound of formula (I), where R¹ represents NHR⁴ wherein R⁴ represents hydrogen atom and all other symbols are as defined above, with di-tert-butoxy carbonyl thiourea or
(b) reacting the compound of formula (I), where R¹ represents NHR⁴ wherein R⁴ represents substituted or unsubstituted group selected from S(O)₂(C1-C20)alkyl or S(O)₂aryl group and all other symbols are as defined above, with guanidine hydrochloride.

35. A process for the preparation of compound of formula (I) 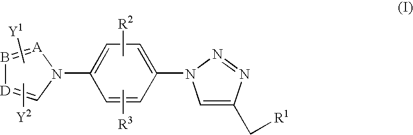
where R¹ represents NHR⁴ where R⁴ represents substituted or unsubstituted group selected from —C(═NH)—(C1-C20)alkyl or —C(═NH)-aryl; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, carboxyl or substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkyl sulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises:
(i) reacting the compound of formula (I) 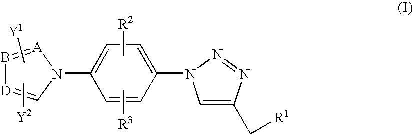
where R¹ repersents NHR⁴, wherein R⁴ represents —C(═S)—NH₂ and all other symbols are as defined above, with di tert-butoxy carbonyl ether [(BOC)₂O], to produce a compound of formula (I), where R¹ represents NHR⁴, wherein R⁴ represents —C(═S)—NH₂ group substituted with tert-butoxy carbonyl group and all other symbols are as defined above and
(ii) reacting the above compound of formula (I), with a compound of formula (Ih)
R⁷—NH₂ (Ih)
where R⁷ represents substituted or unsubstituted (C1-C20)alkyl or aryl group, to produce a compound of formula (I) where R¹ represents NHR⁴ where R⁴ represents substituted or unsubstituted group selected from —C(—NH)—(C1-C20)alkyl or —C(═NH)-aryl group and all other symbols are as defined above.

36. A process for the preparation of compound of formula (I) 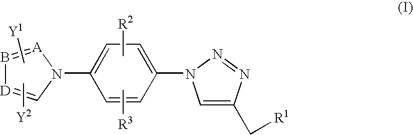
where R¹ represents halogen atom; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, carboxyl or substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkyl sulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkyl amino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises: reacting the compound of formula (I) where R¹ represents hydroxy group and all other symbols are as defined above, with SOCl₂, PCl₅/PBr₃, tetrahalomethane, in the presence of PPh₃ or P(alkyl)₃.

37. A process for the preparation of compound of formula (I) 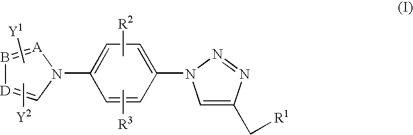
where R¹ represents ‘SH’ group; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, carboxyl or substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises:
(i) reacting the compound of formula (I) where R¹ represents halogen atom, to produce a compound of formula (Ii), 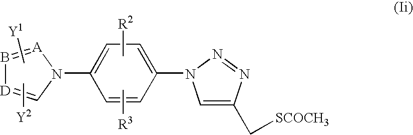
where all other symbols are as defined above, with a base and thiolacetic acid,
(ii) reacting the compound of formula (Ii), to produce a compound of formula (I) where R¹ represents ‘SH’ group and all other symbols are as defined above, with base.

38. A process for the preparation of compound of formula (I) 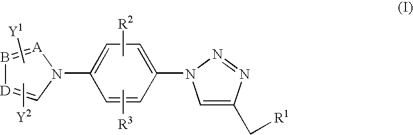
where R¹ represents NHR⁴, wherein R⁴ represents —S(O)₂(C1-C20)alkyl, —S(O)₂aryl group; R² and R³ may be same or different and independently represent hydrogen, halogen atom, substituted or unsubstituted (C1-C20)alkyl group, halo(C1-C20)alkyl, (C1-C20)alkoxy, aryl, heteroaryl, cyano, nitro, OR^{a} where R^{a} represents substituted or unsubstituted (C1-C20)alkyl group; Y¹ and Y² may be same or different and independently represent hydrogen, halogen, cyano, nitro, formyl, hydroxy, amino, carboxyl or substituted or unsubstituted groups selected from (C1-C20)alkyl, hydroxy(C1-C20)alkyl, dihydroxy(C1-C20)alkyl, (C1-C20)alkoxy(C1-C20)alkyl, aminocarbonyl, (C1-C20)alkylcarbonyl, (C1-C20)alkoxycarbonyl, carboxy(C1-C20)alkyl, (C1-C20)alkylsulfonyl, (C1-C20)alkylcarbonylamino(C1-C20)alkyl, arylcarbonylamino(C1-C20)alkyl, (C1-C20)alkylaminocarbonyl, (C1-C20)alkylcarbonyloxy(C1-C20)alkyl, amino(C1-C20)alkyl, mono(C1-C20)alkylamino, di(C1-C20)alkylamino, arylamino, (C1-C20)alkoxy, aryl, aryloxy, aralkyl, heteroaryl, heteroaralkyl, heterocyclyl, heterocycloalkyl or heteroaralkenylaminoalkyl; or any one or two of Y¹ or Y² may represent substituted or unsubstituted —CH═NOR′″, wherein R′″ represents hydrogen, (C1-C20)alkyl, (C1-C20)alkoxy, aryl, heteroaryl and aralkyl group, carboxylic acid or its derivatives; A, B and D independently represent N or —CH, which comprises: reacting the compound of formula (I), where R¹ represents NHR⁴ wherein R⁴ represents hydrogen atom.

39. A pharmaceutical composition comprising a compound of formula (I) 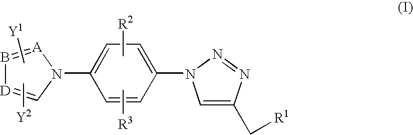
as claimed in claim 1 and a pharmaceutically acceptable carrier, diluent, excipient or solvate.

40. The pharmaceutical composition as claimed in claim 39, in the form of a tablet, capsule, powder, syrup, solution or suspension.

41. A method of treating or preventing a bacterial infection comprising administering a therapeutically effective amount of a compound of formula (I) as claimed in claim 1 to a patient in need thereof.

42. A method of treating or preventing a bacterial infection comprising administering a therapeutically effective amount of a pharmaceutical composition as claimed in claim 39 or 40, to a patient in need thereof.

43. A composition comprising a compound as claimed in claim 12 and a pharmaceutically acceptable carrier, diluent, excipient or solvate.

44. The pharmaceutical composition as claimed in claim 43, in the form of a tablet, capsule, powder, syrup, solution or suspension.

45. A method of treating or preventing a bacterial infection comprising administering a therapeutically effective amount of a compound as claimed in claim 12 to a patient in need thereof.

46. A method of treating or preventing a bacterial infection comprising administering a therapeutically effective amount of a composition as claimed in claim 43 or 44 to a patient in need thereof.
